(19) United States
(12) Patent Application Publication
(10) Pub. No.: US 2026/0232837 A1
(43) Pub. Date: Aug. 13, 2026

(54) LIPID NANOPARTICLE WITH NON-COVALENT BIFUNCTIONAL BINDERS FOR ACTIVE TARGETING

(71) Applicant: FAPON MOTE (SHENZHEN) CO., LTD., Shenzhen, Guangdong (CN)

(72) Inventors: Robert Alexander WESSELHOEFT, Lexington, MA (US); Amit SINGH, Lexington, MA (US); Zhiwei LUO, Dongguan, Guangdong (CN); Feng LIN, Dongguan, Guangdong (CN); Ting LAN, Dongguan, Guangdong (CN); Sanpeng LI, Dongguan, Guangdong (CN)

(21) Appl. No.: 19/580,936

(22) PCT Filed: Oct. 23, 2024
(86) PCT No.: PCT/CN2024/126876

§ 371 Date: Apr. 22, 2026

Related U.S. Application Data
(60) Provisional application No. 63/592,915, filed on Oct. 24, 2023.

(51) Int. Cl. A61K 47/69; A61K 31/7105; A61K 47/68; C12N 15/88

(52) U.S. Cl. CPC A61K 47/6913; A61K 31/7105; A61K 47/6879; A61K 47/6929; C12N 15/88

(57) ABSTRACT

A nanoparticle complex comprises a bifunctional binder capable of non-covalently binding to the lipid nanoparticle in the nanoparticle complex.

### CROSS-REFERENCE TO RELATED APPLICATIONS

This application claims priority to U.S. provisional application 63/592,915, filed Oct. 24, 2023, the disclosure of which is incorporated herein by reference.

### SEQUENCE LISTING

The sequence listing that is contained in the file named “088177-8001WO01”, which is 75,282 bytes (as measured in Microsoft Windows) and was created on Oct. 22, 2024, is filed herewith by electronic submission and is incorporated by reference herein.

### FIELD OF THE INVENTION

The present disclosure generally relates to lipid nanoparticles. In particular, the present disclosure relates to a lipid nanoparticle having non-covalent bifunctional binders for active targeting, and uses thereof.

### BACKGROUND

Liposomes are synthetic lipid vesicles, which can encapsulate a variety of sizes of molecules in their internal spaces. Liposomes have been widely studied as carriers for delivering substances to cells and tissues in the body. (Gregoriadis, Liposome Technology, Vol. I, II, III, CRC Press, Boca Raton, 1985.) Nanoparticles, like liposomes, also have been studied for use as drug carriers, especially PEG-NPs, which can be obtained by covalent attachment of poly(ethylene glycol) (PEGylation) to nanoparticles and thus displays an increased drug bioavailability, enhanced blood circulation half-life and hinder capture by the reticuloendothelial system (RES). However, NPs, especially PEG-NPs often accumulate near tumors but do not penetrate into the tumor mass, and some drugs cannot easily diffuse from PEG-NPs to target cells. Although several studies have provided some ideas to increase specific targeting and intracellular uptake, there still exists some other issues to be resolved, such as manufacturability of targeted lipid nanoparticles, which are currently covalently functionalized, blood clearance of particles resulting in loss of efficacy in the presence of particle-binding antibodies, and particle targeting to extrahepatic tissues and cell types etc. Besides, only a narrow spectrum of diseases can be treated is also a pending issue. Taking the technical solution published in WO2015134411(A1) as an example, the upgraded NPs disclosed therein can only deliver small molecules, and thus can treat cancer only. Therefore, there exists in the related field, a need for a product which can overcome the forgoing deficiencies.

### SUMMARY OF THE INVENTION

Throughout the present disclosure, the articles “a,” “an,” and “the” are used herein to refer to one or to more than one (i.e., to at least one) of the grammatical object of the article. By way of example, “an antibody” means one antibody or more than one antibody.

In one respect, the present disclosure provides a nanoparticle complex comprising (a) a lipid nanoparticle (LNP) comprising a surface component; (b) a bifunctional binder comprising (i) a self-binder moiety non-covalently binding to the surface component, (ii) a target-binder moiety capable of binding to a targeted cell-surface component of a target cell, wherein the self-binder moiety is linked to the target-binder moiety; and (c) a cargo carried by the lipid nanoparticle, wherein the cargo is one or more nucleic acid molecules.

In some embodiments, the nucleic acid is DNA or RNA. In some embodiments, the nucleic acid is selected from a group consisting of a dsDNA, a ssDNA, a circular RNA, a mRNA, a noncoding RNA, a dsRNA, a miRNA, an siRNA and a tRNA; preferably a circular RNA, a mRNA or an siRNA.

In some embodiments, the lipid nanoparticle comprises an ionizable lipid, a helper lipid, a PEG-modified lipid, and a cholesterol-based lipid, or other lipids with similar functions.

In some embodiments, the lipid nanoparticle comprises the biotin-modified lipoid, such as biotin-modified cholesterol, or other modified lipoids with similar functions.

In some embodiments, ionizable lipid is a cationic lipid selected from HGT4003, HGT5000, HGT5001, HGT5002, DOTMA, DOGS, DOSPA, DOTAP, DODAP, DOTMA, DSDMA, DODMA, DLinDMA, DLenDMA, DODAC, DDAB, DMRIE, CLinDMA, CpLinDMA, DMOBA, DOcarbDAP, DLinDAP, DLincarbDAP, DLinCDAP, DLin-K-XTC2-DMA, DLin-KC2-DMA, AA3-DLinXTC, ALNY-100, NC98-5, C12-200, MC2, MC3, MC4, ALC-0315, SM-102, ATX-001, ATX-100, Lipid 2, Lipid 5, 9A1P9, OF-Deg-Lin, 80-O16B, 93-O17S, 93-O17O, 306-O12B, 113-O16B, 306Oi10, 113-O12B, cKK-E12, 98N12-5, OF-02, TLC053, LPO1, BAMEA-016B, CL1, BP Lipid 310, Lipid A9, L319, and ICE.

In some embodiments, the helper lipid is a non-cationic lipid selected from DSPC, DOPC, DPPC, DOPG, DPPG, DOPE, POPC, POPE, POPG, DEPE, Egg sphingomyelin, DOPE-mal, DPPE, DMPE, DSPE, 16-O-monomethyl PE, 16-O-dimethyl PE, 18-1-trans PE and SOPE.

In some embodiments, the PEG-modified lipid is a polyethylene glycol chain of up to 10 kDa in length covalently attached to a lipid with alkyl chain(s) of C6-C20 length. In some embodiments, the PEG-modified lipid is a glycol (PEG)-modified phospholipid or ceramide.

In some embodiments, the surface component is an ionizable lipid, a helper lipid, a PEG-modified lipid, a PEG, a cholesterol-based lipid, other lipids with similar functions, or any component present on the surface of the lipid nanoparticle. In some embodiments, the surface component is any component that can triggers an immune response present on the surface of the lipid nanoparticle, such as PEG.

In some embodiments, the surface component is the biotin-modified lipoid, such as biotin-modified cholesterol, or other modified lipoids with similar functions.

In some embodiments, the self-binder moiety is a first antigen binding fragment specifically binding to the surface component.

In some embodiments, the first antigen binding fragment specifically binds to PEG.

In some embodiments, the first antigen binding fragment is derived from an IgG, an IgA, an IgM, an IgE or an IgD. In some embodiments, the first antigen binding fragment is a Fab, a VHH antibody, an scFv, an scFab, or a diabody.

In some embodiments, the target-binder moiety is an Fc or a fragment thereof of an IgG, an IgA, an IgM, an IgE or an IgD.

In some embodiments, the targeted cell-surface component of the target cell is a protein, a glycoRNA, a lipid or lipid raft. In some embodiments, the protein is a receptor. In some embodiments, the receptor is an Fc receptor, FcgR1, FcgR2, FCgr3, FcgR4, FceR1, FceR2, FcaR1, FcuR, FcdR, or an isoform thereof, or C1qR or FcRn.

In some embodiments, the target cell is an antigen presenting cell, a monocyte, a neutrophil, a macrophage, a dendritic cell, a mast cell, a T cell, a natural killer cell, a Kupffer cell, a B cell, or a tumor cell.

In some embodiments, the target-binder moiety is a second antigen binding fragment specifically binding to the receptor (e.g., any cell type specific surface proteins, any tumor associated antigens, etc.). In some embodiments, the second antigen binding fragment is a Fab, a VHH antibody, an scFv, an scFab, or a diabody. In some embodiments, the receptor is selected from the group consisting of CD2, CD3, CD4, CD5, CD7, CD8, CD25, CD45RA, CD45RO, PDL1 and CTLA4.

In some embodiments, the T cell is a CD3+ T cell, a CD4+ T cell, a CD8+ T cell, a T regulatory cell, a cytotoxic T cell, or a helper T cell.

In some embodiments, the self-binder moiety is covalently linked to the target-binder moiety. In some embodiments, the self-binder moiety is linked to the target-binder moiety via a linker. In some embodiments, the linker comprises an Fc or a fragment thereof. In some embodiments, the linker comprises a multimerization domain. In some embodiments, the self-binder moiety is non-covalently linked to the target-binder moiety.

In some embodiments, the nanoparticle complex has at least one of the functions as following, compared to a reference nanoparticle complex: (a) substantially avoids uptake by hepatocytes, (b) substantially contacts cells displaying the targeted cell-surface component of the target cell, and (c) substantially decreases accelerated blood clearance of the nanoparticle complex, wherein the reference nanoparticle complex is a nanoparticle complex without the bifunctional binder.

In one respect, the present disclosure provides a pharmaceutical composition, comprising the nanoparticle complex of this invention and a pharmaceutically acceptable carrier.

In another aspect, the present disclosure provides a method of delivering a cargo molecule to a target cell in a subject, comprising administering to the subject the nanoparticle complex of this invention.

In some embodiments, the nanoparticle complex is administered intravenously, intra-arterially, intra-tumorally, orally, sublingually, rectally, vaginally, transmucosally, via pulmonary system including intratracheally or via inhale, parenterally (including intradermally, transdermally, intramuscularily, subcutaneously), via intramedullary injection, intrathecally, intraventricularily, intraperitoneally, or intranasally.

### BRIEF DESCRIPTION OF THE DRAWINGS

FIG. 1 shows the general structures and the nomenclatures of the bifunctional binders.

FIG. 2 shows an exemplary structure of the bifunctional binder.

FIG. 3 shows an exemplary structure of the bifunctional binder.

FIG. 4 shows several exemplary structures of the bifunctional binder.

FIG. 5 shows several exemplary structures of the bifunctional binder.

FIG. 6 shows several exemplary structures of the bifunctional binder.

FIG. 7 shows an exemplary structure of the bifunctional binder.

FIG. 8 shows several exemplary structures of the bifunctional binder.

FIG. 9A shows the bar graph of the size and PDI of the naked LNP and RM105 modified LNPs frozen and stored at −80 C for 7 days.

FIG. 9B shows the bar graph of the EE, size and PDI of the naked LNP and anti-biotin modified LNPs.

FIG. 10A shows the IVIS images of RM105 (PEG binder) mediated liver detargeting in mice.

FIG. 10B shows the bar chart of total Flux shown in FIG. 10A.

FIG. 10C shows the IVIS images of RM105 (PEG binder) mediated liver detargeting in isolated liver.

FIG. 10D shows the bar chart of total Flux shown in FIG. 10C.

FIG. 10E shows the IVIS images of anti-Biotin (cholesterol-biotin binder) mediated liver detargeting in mice.

FIG. 10F shows the bar chart of total Flux shown in FIG. 10E.

FIG. 10G shows the IVIS images of anti-Biotin (cholesterol-biotin binder) mediated liver detargeting in isolated liver.

FIG. 10H shows the bar chart of total Flux shown in FIG. 10G.

FIG. 101 shows the IVIS images of whole-body expression profile of naked and RM105 modified LNP-RNA complexes with and without freeze/thaw(F/T).

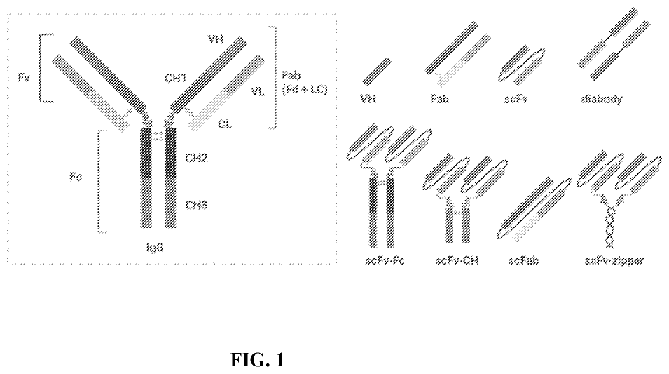

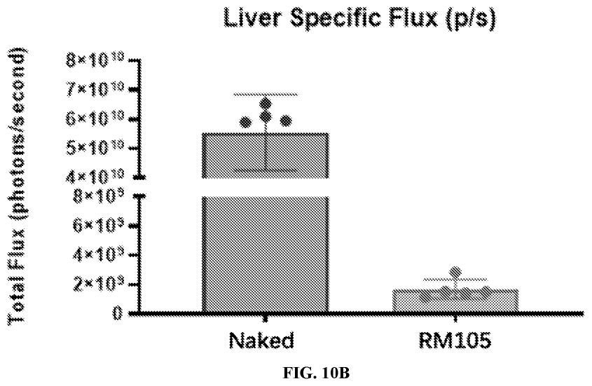

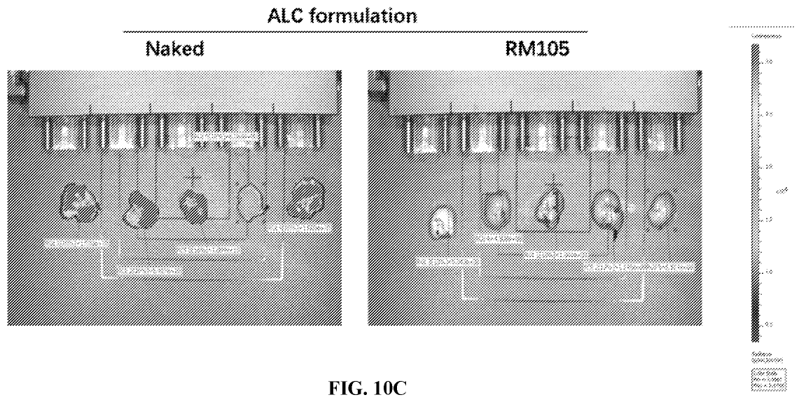

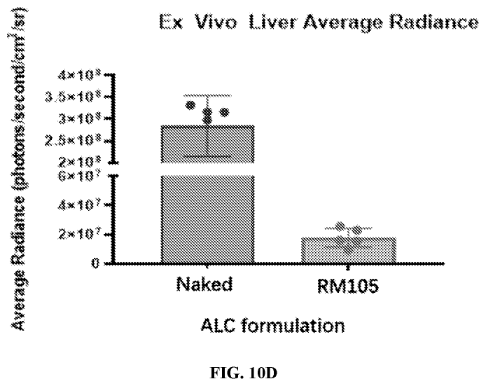

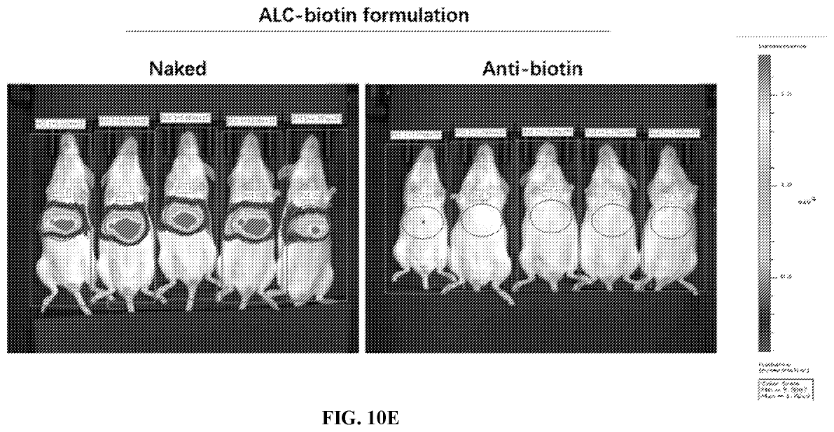

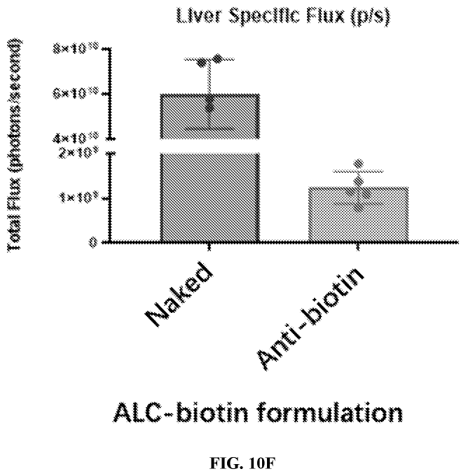

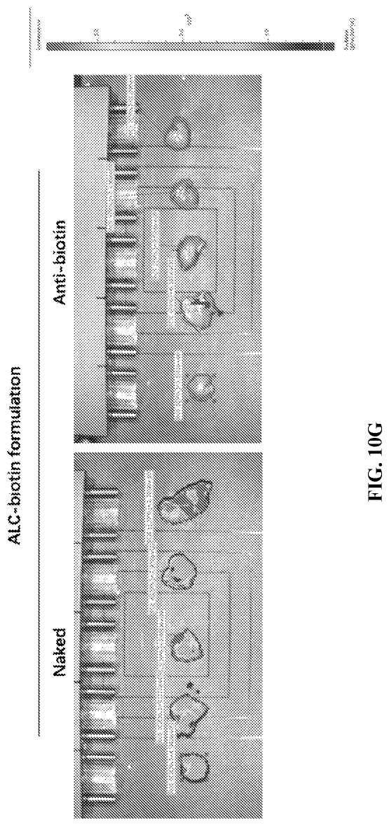

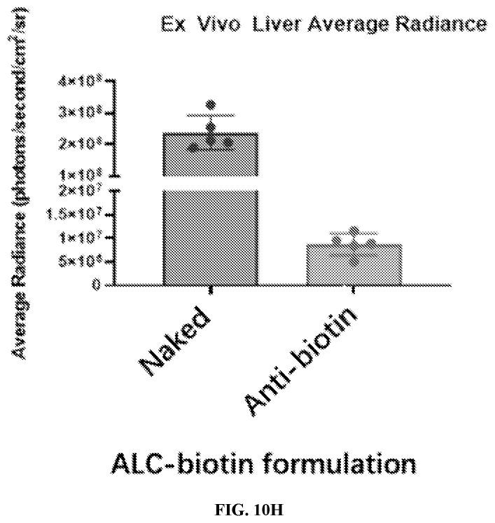

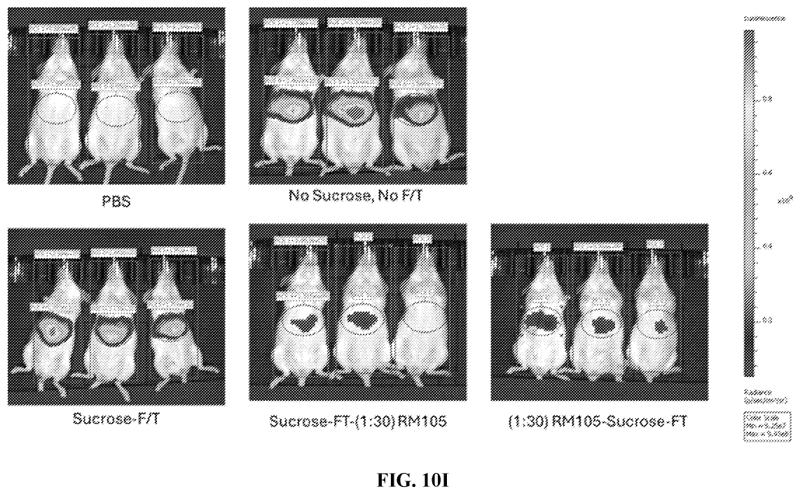

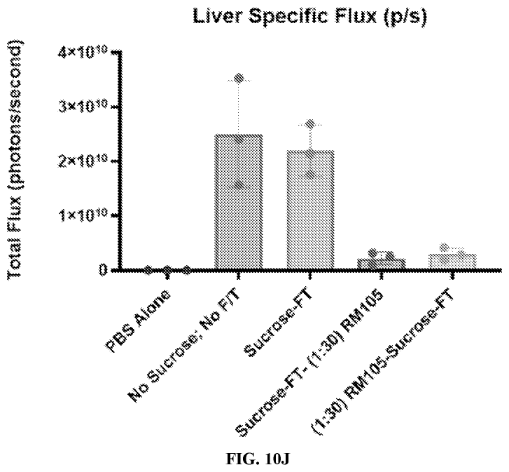

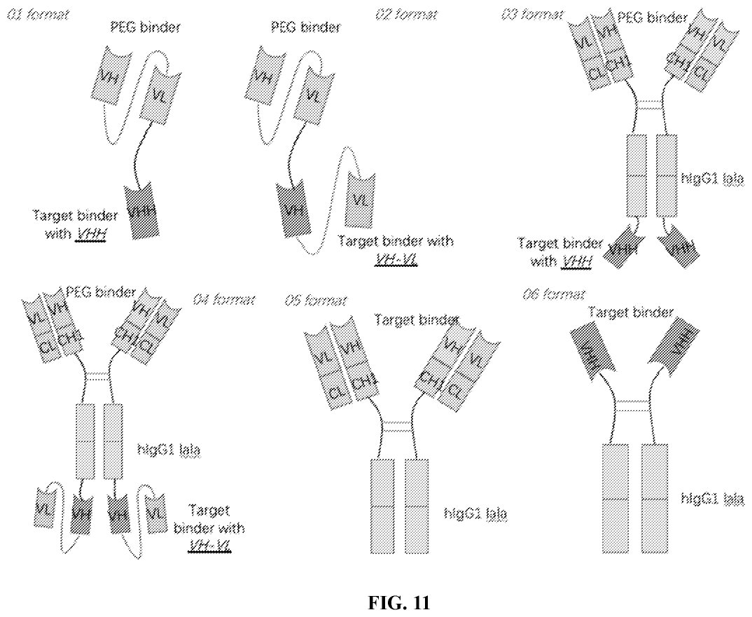

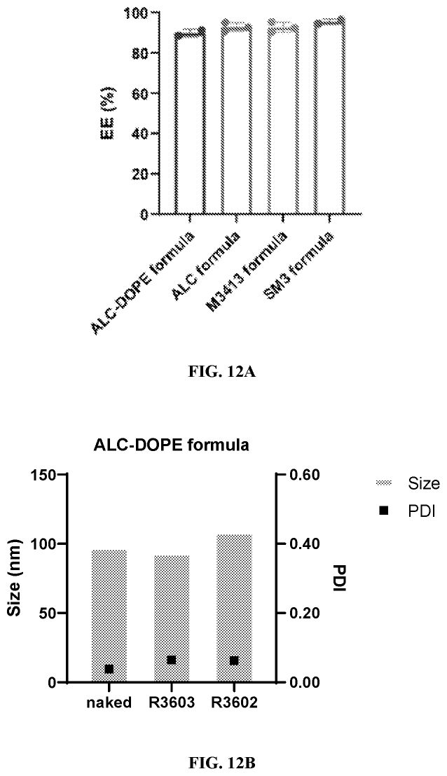

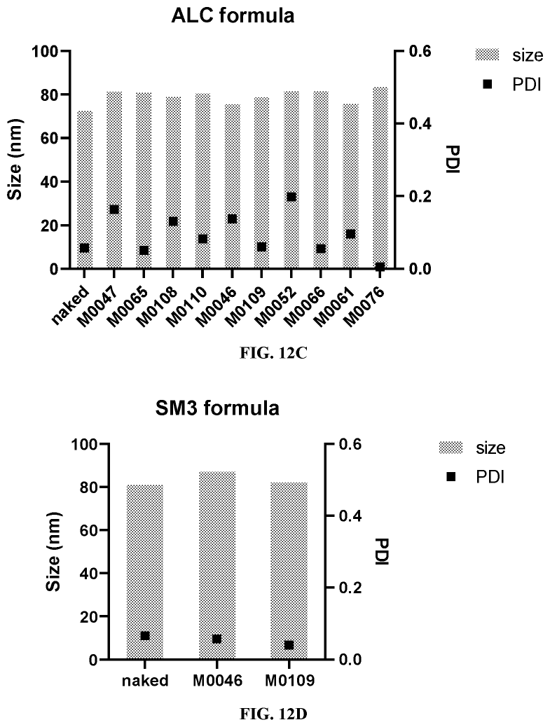

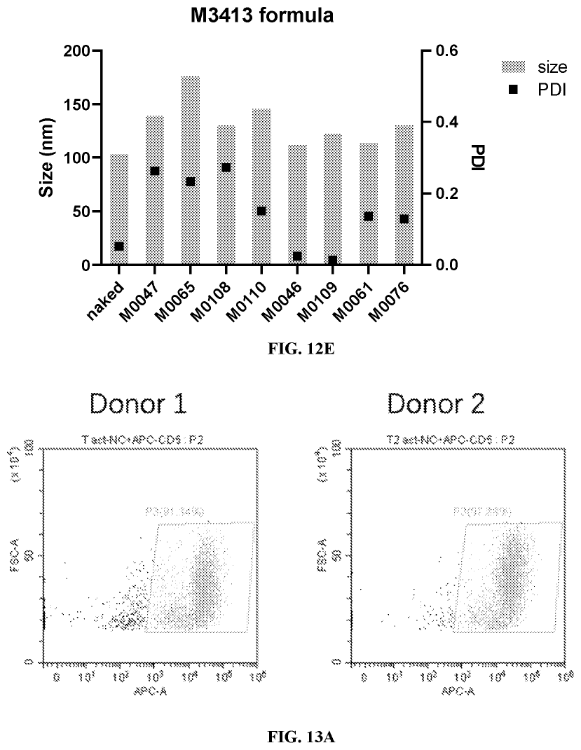

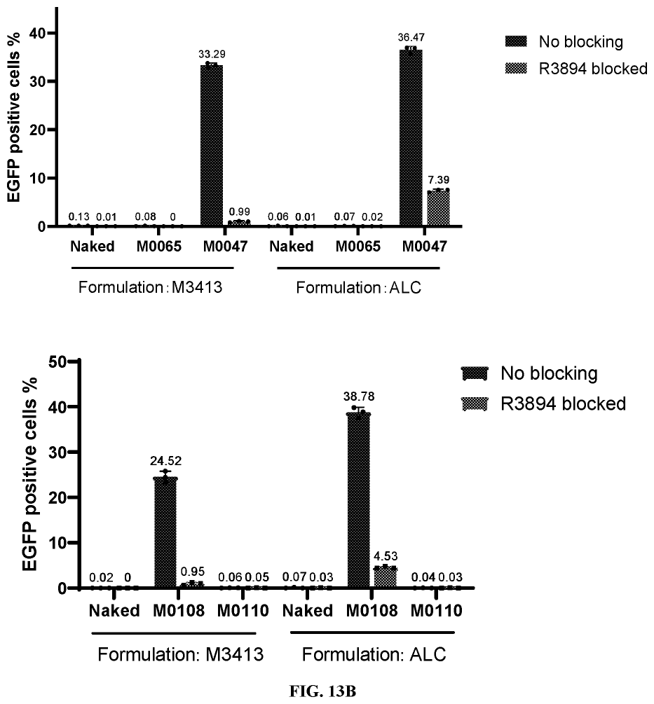

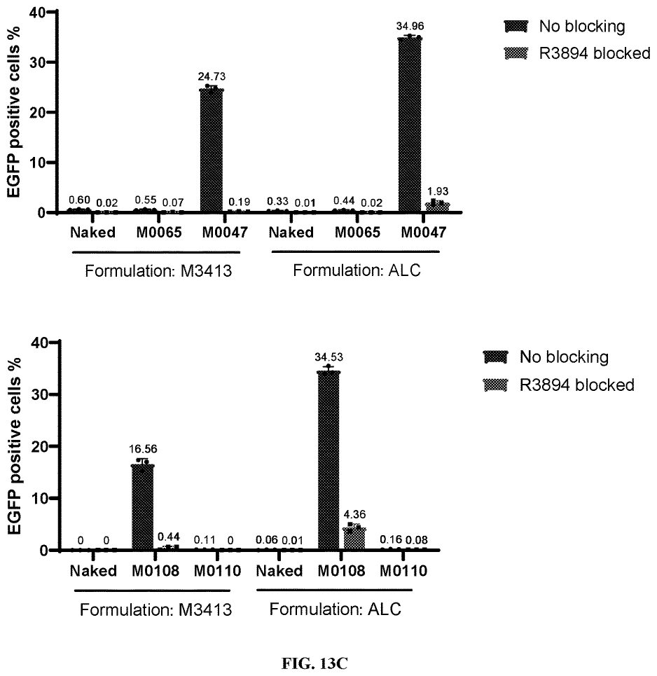

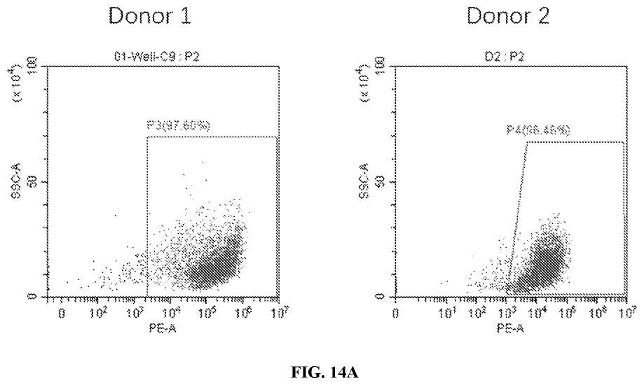

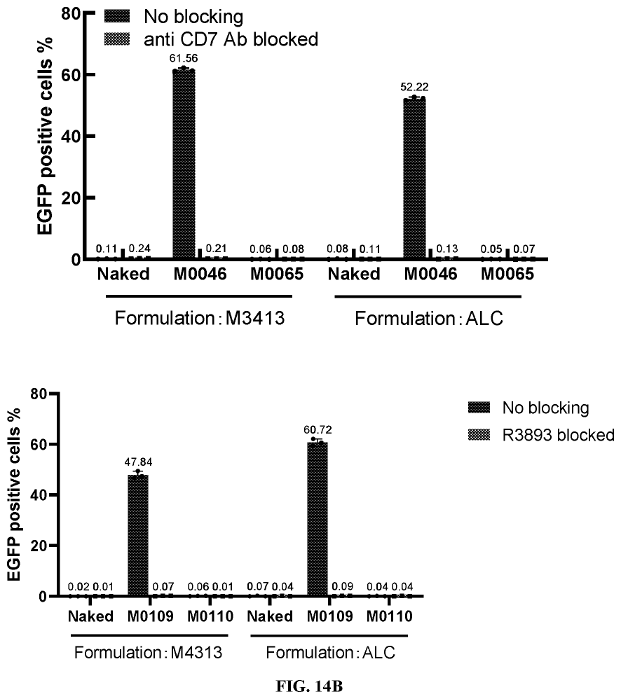

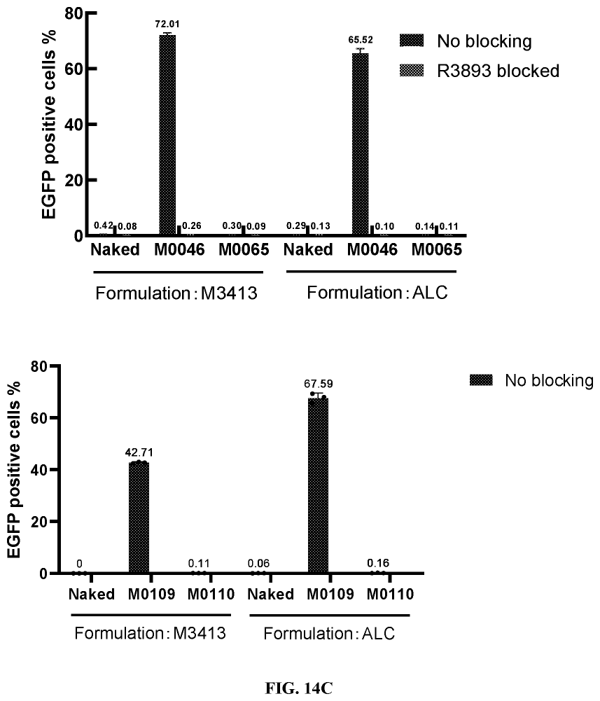

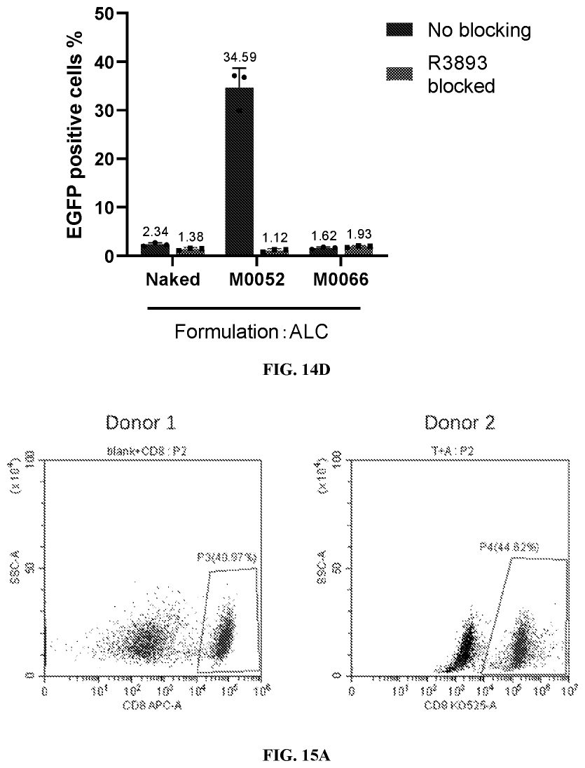

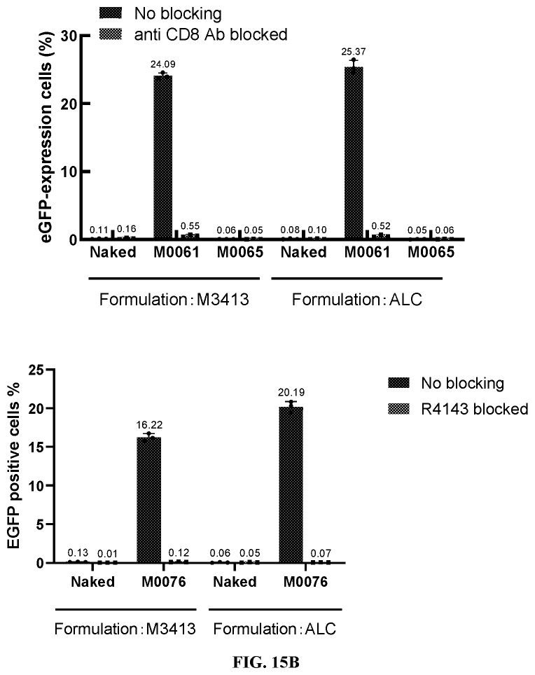

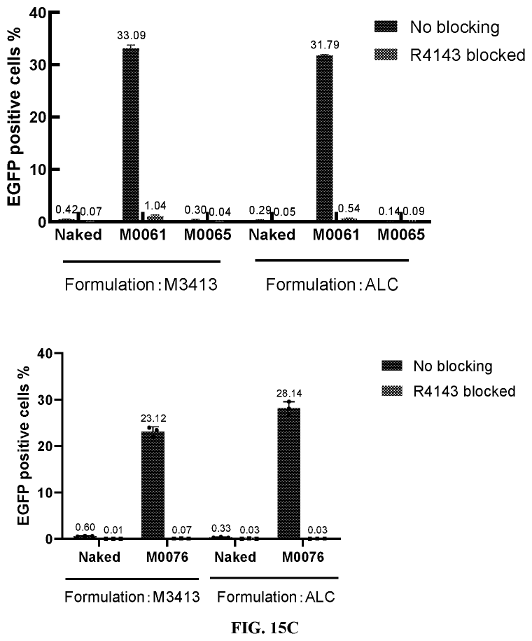

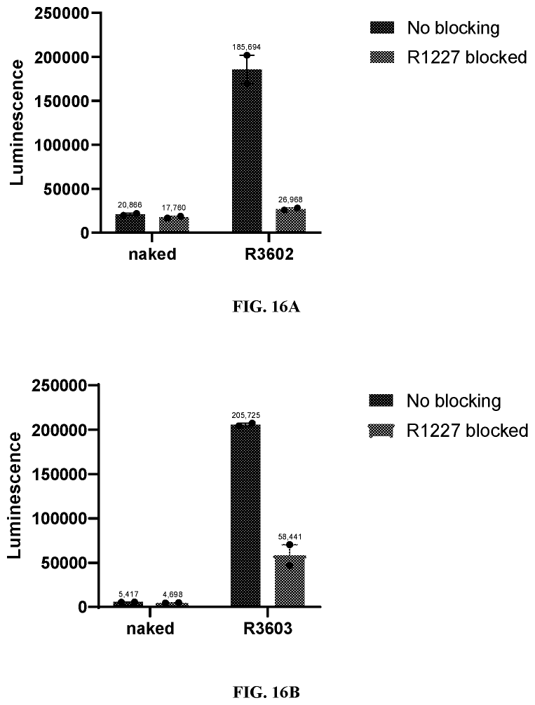

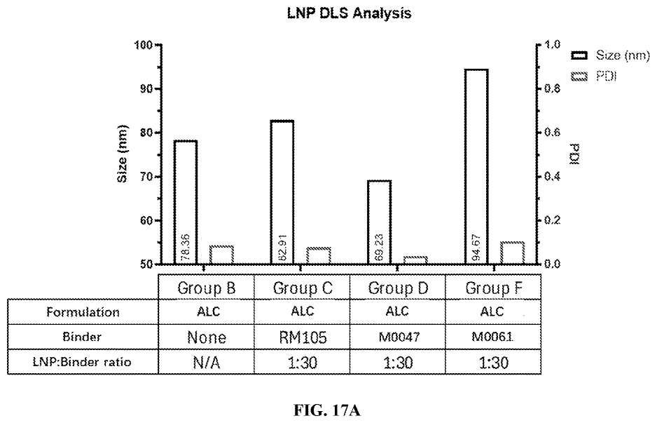

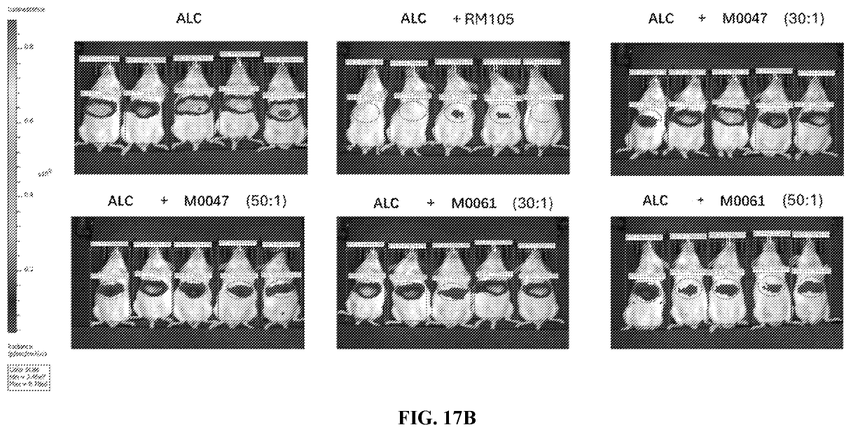

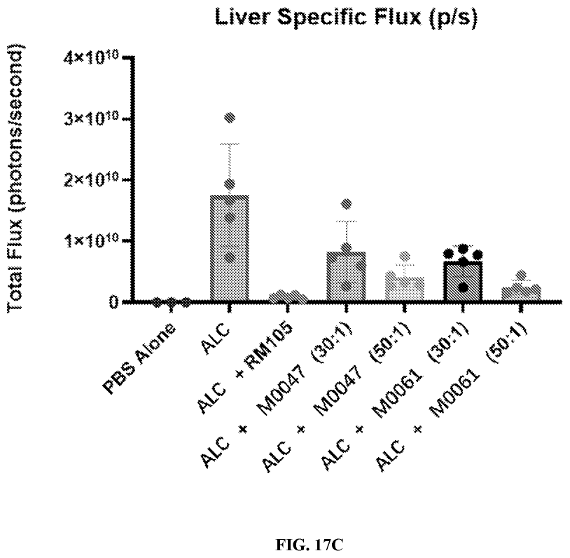

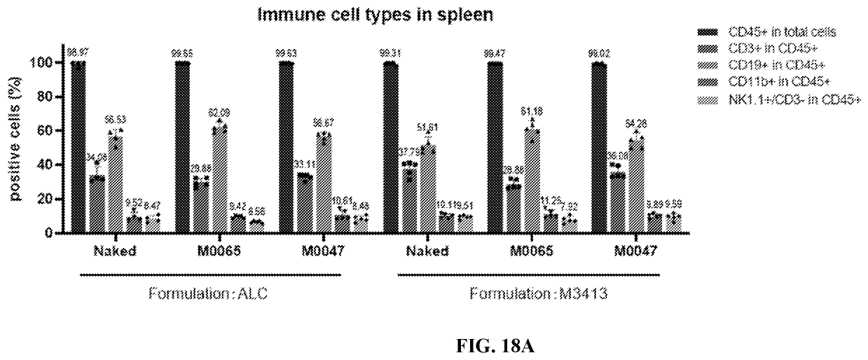

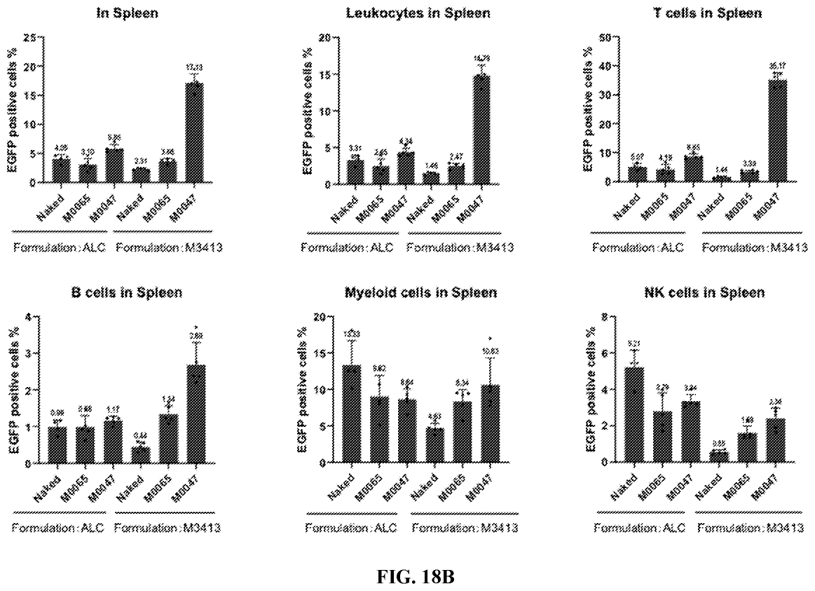

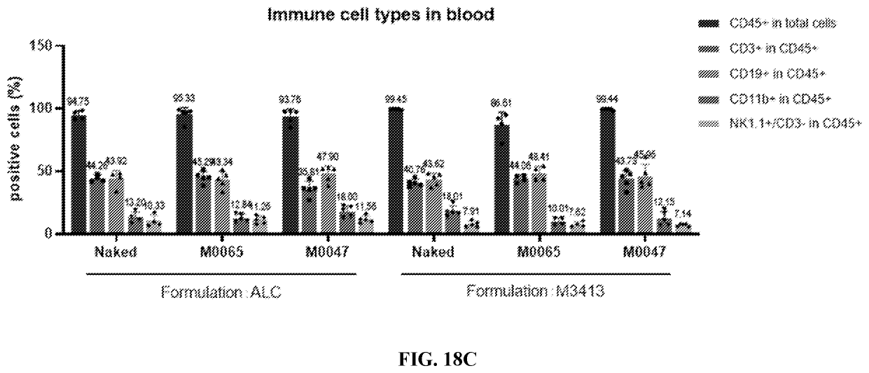

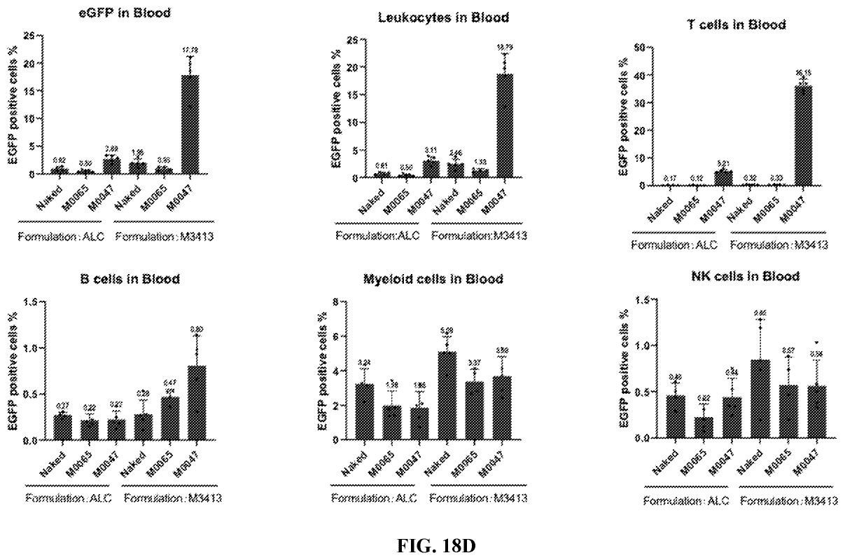

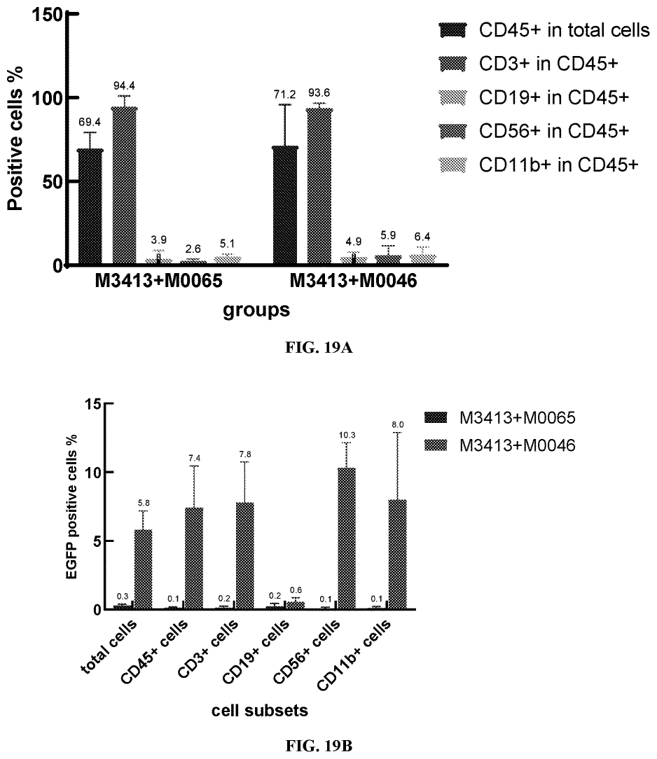

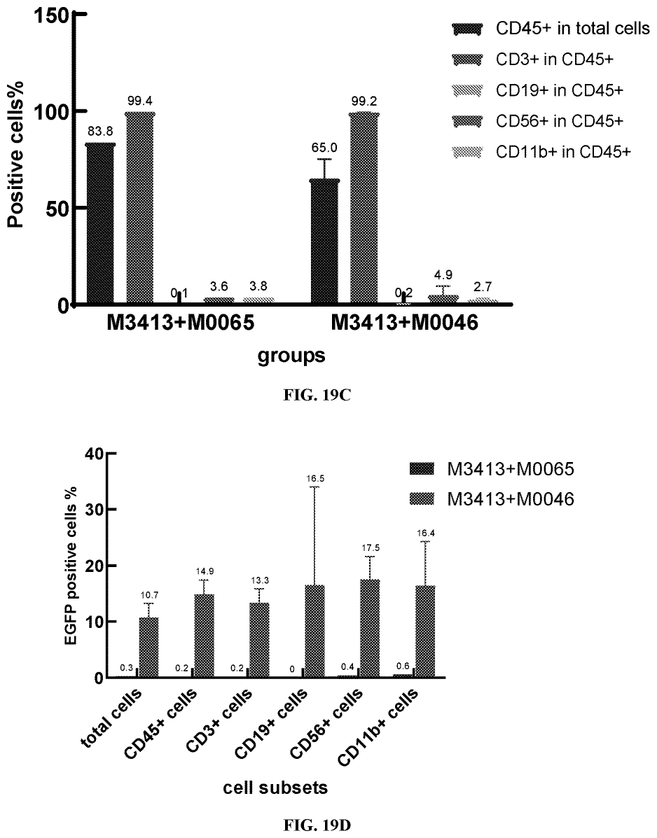

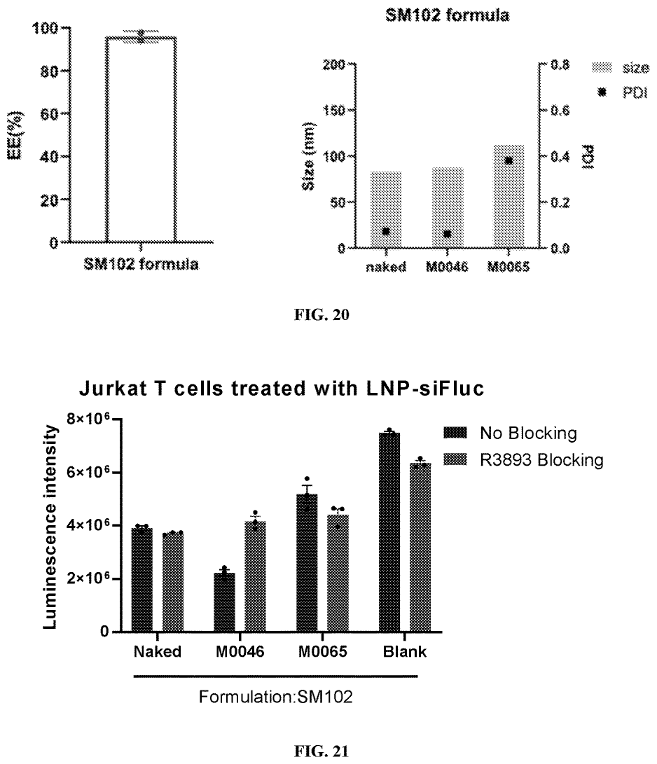

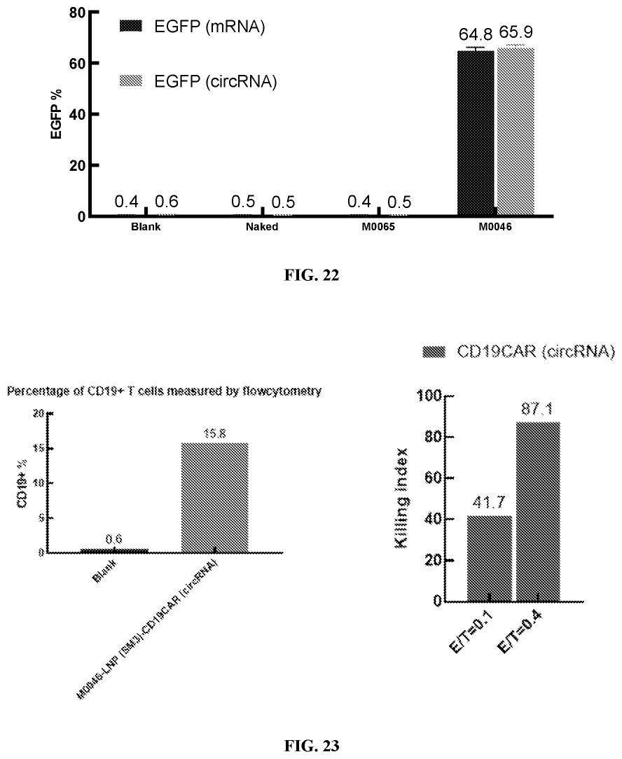

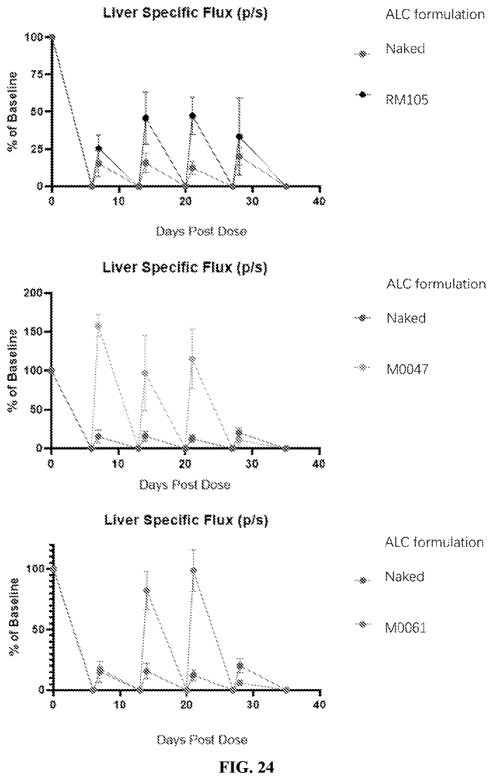

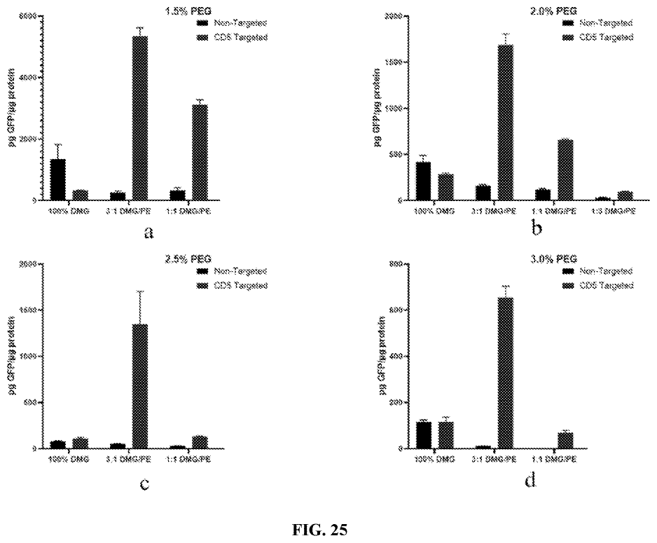

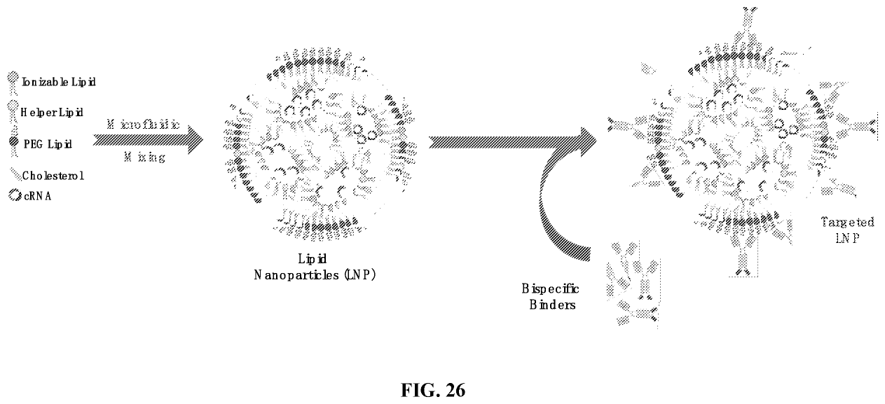

FIG. 10J shows the bar chart of total Flux shown in FIG. 101.

FIG. 11 shows the schematics of binder formats.

FIG. 12A shows the bar chart of EE of naked LNP in indicated formulations

FIGS. 12B-12E show the bar chart of size and PDI of naked LNP and LNP modified with indicated binders.

FIG. 13A shows the plots of flow cytometry measuring CD5+ percentage in isolated T cells from donor 1 and 2.

FIG. 13B shows the bar chart of flow cytometry measuring M0047 and M0108 (CD5xPEG bispecific binders) mediated delivery in isolated T cells of donor 1.

FIG. 13C shows the bar chart of flow cytometry measuring M0047 and M0108 (CD5xPEG bispecific binders) mediated delivery in isolated T cells of donor 2.

FIG. 14A shows the bar chart of flow cytometry measuring CD7+ percentage in isolated T cells from donor 1 and 2.

FIG. 14B shows the bar chart of flow cytometry measuring M0046 and M0109 (CD7xPEG bispecific binders) mediated delivery in isolated T cells of donor 1.

FIG. 14C shows the bar chart of flow cytometry measuring M0046 and M0109 (CD7xPEG bispecific binders) mediated delivery in isolated T cells of donor 2.

FIG. 14D shows the bar chart of flow cytometry measuring M0052 (02 format CD7XPEG bispecific binder) mediated delivery in Jurkat cells.

FIG. 15A shows the bar chart of flow cytometry measuring CD8+ percentage in isolated T cells from donor 1 and 2.

FIG. 15B shows the bar chart of flow cytometry measuring M0061 and M0076 (CD8xPEG bispecific binders) mediated delivery in isolated T cells of donor 1.

FIG. 15C shows the bar chart of flow cytometry measuring M0061 and M0076 (CD8xPEG bispecific binders) mediated delivery in isolated T cells of donor 2.

FIG. 16A shows the bar chart of luminescence level measuring R3602 and R3603 (PDL1xPEG bispecific binders) mediated delivery in MC38-PD-L1.

FIG. 16B shows the bar chart of luminescence level measuring R3602 and R3603 (PDL1xPEG bispecific binders) mediated delivery in MC38-PD-L1.

FIG. 17A shows the bar chart of size and PDI of ALC formulation as naked (Group B) and binder coated (Group C-F),

FIG. 17B shows the IVIS images to demonstrate bispecific binders mediated liver detargeting in mice.

FIG. 17C shows the bar chart showing the liver specific flux level from IVIS images in FIG. 17B.

FIG. 18A shows the bar chart of flow cytometry measuring cell identity in spleen of hCD5 mice.

FIG. 18B shows the bar chart of flow cytometry measuring M0047 (CD5xPEG bispecific binder) mediated delivery in spleen of hCD5 mice.

FIG. 18C shows the bar chart of flow cytometry measuring cell identity in PBMC of hCD5 mice.

FIG. 18D shows the bar chart of flow cytometry measuring M0047 (CD5xPEG bispecific binder) mediated delivery in PBMC of hCD5 mice.

FIG. 19A shows the bar chart of flow cytometry measuring cell identity in spleen of hPBMC-mice.

FIG. 19B shows the bar chart of flow cytometry measuring M0046 (CD7xPEG bispecific binder) mediated delivery in spleen of hPBMC-mice.

FIG. 19C shows the bar chart of flow cytometry measuring cell identity in PBMC of hPBMC-mice.

FIG. 19D shows the bar chart of flow cytometry measuring M0046 (CD7xPEG bispecific binder) mediated delivery in PBMC of hPBMC-mice.

FIG. 20 shows the bar chart of EE, particle size and PDI of naked LNP-siFluc and binder-LNP-siFluc.

FIG. 21 shows the bar chart of luminescence levels measuring knockdown efficiency of naked and M0046 (CD7xPEG bispecific binder) modified LNP-siFluc in Jurkat-Fluc cells.

FIG. 22 shows the bar chart of flow cytometry measuring CD7xPEG binder mediated delivery of mRNA and circRNA CD19CAR in vitro.

FIG. 23 shows the bar chart of in vitro cytotoxicity assay of CAR-T produced from M0046-LNP (SM3)-CD19CAR (circRNA).

FIG. 24 shows the relative liver specific flux level of naked and indicated binders modified LNP-Fluc over redosing.

FIG. 25 illustrates the relative in vitro expression of GFP when the SM102 based formulation at 1.5-3% total PEG content were administered at varying ratios of sheddable (DMG-PEG) to non-sheddable (PE-PEG), DMG=DMG-PEG, PE=DSPE-PEG.

FIG. 26 shows the schematic illustrating the non-covalent modification of the LNP surface with the bispecific binder to generate tissue-specific targeted LNPs.

### DETAILED DESCRIPTION OF THE INVENTION

The following description of the disclosure is merely intended to illustrate various embodiments of the disclosure. As such, the specific modifications discussed are not to be construed as limitations on the scope of the disclosure. It will be apparent to a person skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

### 1. Definitions

The term “antibody” as used herein includes any immunoglobulin, monoclonal antibody, polyclonal antibody, multivalent antibody, bivalent antibody, monovalent antibody, multi-specific antibody, or bispecific antibody that binds to a specific antigen. A native intact antibody comprises two heavy (H) chains and two light (L) chains. Mammalian heavy chains are classified as alpha, delta, epsilon, gamma, and mu, each heavy chain consists of a variable region (VH) and a first, second, third, and optionally fourth constant region (CH1, CH2, CH3, CH4 respectively); mammalian light chains are classified as X or x, while each light chain consists of a variable region (VL) and a constant region. The antibody has a “Y” shape, with the stem of the Y consisting of the second and third constant regions of two heavy chains bound together via disulfide bonding. Each arm of the Y includes the variable region and first constant region of a single heavy chain bound to the variable and constant regions of a single light chain. The variable regions of the light and heavy chains are responsible for antigen binding. The variable regions in both chains generally contain three highly variable loops called the complementarity determining regions (CDRs) (light chain CDRs including LCDR1, LCDR2, and LCDR3, heavy chain CDRs including HCDR1, HCDR2, HCDR3). CDR boundaries for the antibodies and antigen-binding fragments disclosed herein may be defined or identified by the conventions of Kabat, IMGT, Chothia, or Al-Lazikani (Al-Lazikani, B., Chothia, C., Lesk, A. M., J. Mol. Biol., 273(4), 927 (1997); Chothia, C. et al., J Mol Biol. December 5; 186(3):651-63 (1985); Chothia, C. and Lesk, A. M., J. Mol. Biol., 196,901 (1987); Chothia, C. et al., Nature. December 21-28; 342(6252):877-83 (1989); Kabat E. A. et al., Sequences of Proteins of immunological Interest, 5th Ed. Public Health Service, National Institutes of Health, Bethesda, Md. (1991); Marie-Paule Lefranc et al., Developmental and Comparative Immunology, 27: 55-77 (2003); Marie-Paule Lefranc et al., Immunome Research, 1(3), (2005); Marie-Paule Lefranc, Molecular Biology of B cells (second edition), chapter 26, 481-514, (2015)). The three CDRs are interposed between flanking stretches known as framework regions (FRs) (light chain FRs including LFR1, LFR2, LFR3, and LFR4, heavy chain FRs including HFR1, HFR2, HFR3, and HFR4), which are more highly conserved than the CDRs and form a scaffold to support the highly variable loops. The constant regions of the heavy and light chains are not involved in antigen-binding, but exhibit various effector functions. Antibodies are assigned to classes based on the amino acid sequences of the constant regions of their heavy chains. The five major classes or isotypes of antibodies are IgA, IgD, IgE, IgG, and IgM, which are characterized by the presence of alpha, delta, epsilon, gamma, and mu heavy chains, respectively. Several of the major antibody classes are divided into subclasses such as IgG1 (gamma1 heavy chain), IgG2 (gamma2 heavy chain), IgG3 (gamma3 heavy chain), IgG4 (gamma4 heavy chain), IgAQ1 (alpha1 heavy chain), or IgA2 (alpha2 heavy chain).

In certain embodiments, the antibody provided herein encompasses any antigen-binding fragments thereof. The term “antigen-binding fragment” as used herein refers to an antibody fragment formed from a portion of an antibody comprising one or more CDRs, or any other antibody fragment that binds to an antigen but does not comprise an intact native antibody structure. Examples of antigen-binding fragment include, without limitation, a diabody, a Fab, a Fab′, a F(ab′)₂, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a bispecific antibody, a multispecific antibody, a camelized single domain antibody, a nanobody, a domain antibody, a bivalent domain antibody, a VHH antibody, and an scFab. An antigen-binding fragment is capable of binding to the same antigen to which the parent antibody binds.

“Fab” with regard to an antibody refers to that portion of the antibody consisting of a single light chain (both variable and constant regions) bound to the variable region and first constant region of a single heavy chain by a disulfide bond.

“Fab′” refers to a Fab fragment that includes a portion of the hinge region.

“F(ab′)₂” refers to a dimer of Fab′.

An “scFab” refers to a single chain Fab.

“Fc” with regard to an antibody (e.g., of IgG, IgA, or IgD isotype) refers to that portion of the antibody consisting of the second and third constant domains of a first heavy chain bound to the second and third constant domains of a second heavy chain via disulfide bonding. Fc with regard to antibody of IgM and IgE isotype further comprises a fourth constant domain. The Fc portion of the antibody is responsible for various effector functions such as antibody-dependent cell-mediated cytotoxicity (ADCC), and complement dependent cytotoxicity (CDC), but does not function in antigen binding.

“Fv” with regard to an antibody refers to the smallest fragment of the antibody to bear the complete antigen binding site. An Fv fragment consists of the variable region of a single light chain bound to the variable region of a single heavy chain. ‘ ’

“Single-chain Fv antibody” or “scFv” refers to an engineered antibody consisting of a light chain variable region and a heavy chain variable region connected to one another directly or via a peptide linker sequence (Huston J S et al. Proc Natl Acad Sci USA, 85:5879(1988)).

“Single-chain Fv-Fc antibody” or “scFv-Fc” refers to an engineered antibody consisting of an scFv connected to the Fc region of an antibody.

“Camelized single domain antibody,” “heavy chain antibody,” or “HCAb” refers to an antibody that contains two V_{H} domains and no light chains (Riechmann L. and Muyldermans S., J Immunol Methods. December 10; 231(1-2):25-38 (1999); Muyldermans S., J Biotechnol. June; 74(4):277-302 (2001); WO94/04678; WO94/25591; U.S. Pat. No. 6,005,079). Heavy chain antibodies were originally derived from Camelidae (camels, dromedaries, and llamas). Although devoid of light chains, camelized antibodies have an authentic antigen-binding repertoire (Hamers-Casterman C. et al., Nature. June 3; 363(6428):446-8 (1993); Nguyen V K. et al. Immunogenetics. April; 54(1):39-47 (2002); Nguyen V K. et al. Immunology. May; 109(1):93-101 (2003)). The variable domain of a heavy chain antibody (VHH domain) represents the smallest known antigen-binding unit generated by adaptive immune responses (Koch-Nolte F. et al., FASEB J. November; 21(13):3490-8. Epub 2007 Jun. 15 (2007)).

A “nanobody” refers to an antibody fragment that consists of a VHH domain from a heavy chain antibody and two constant domains, CH2 and CH3.

A “diabody” or “dAb” includes small antibody fragments with two antigen-binding sites, wherein the fragments comprise a V_{H} domain connected to a V_{L} domain in the same polypeptide chain (V_{H}-V_{L} or V_{L}—V_{H}) (see, e.g., Holliger P. et al., Proc Natl Acad Sci USA. July 15; 90(14):6444-8 (1993); EP404097; WO93/11161). By using a linker that is too short to allow pairing between the two domains on the same chain, the domains are forced to pair with the complementary domains of another chain, thereby creating two antigen-binding sites. The antigen-binding sites may target the same or different antigens (or epitopes). In certain embodiments, a “bispecific ds diabody” is a diabody target two different antigens (or epitopes).

A “domain antibody” refers to an antibody fragment containing only the variable region of a heavy chain or the variable region of a light chain. In certain instances, two or more V_{H} domains are covalently joined with a peptide linker to create a bivalent or multivalent domain antibody. The two V_{H} domains of a bivalent domain antibody may target the same or different antigens.

The term “valent” as used herein refers to the presence of a specified number of antigen binding sites in a given molecule. The term “monovalent” refers to an antibody or an antigen-binding fragment having only one single antigen-binding site; and the term “multivalent” refers to an antibody or an antigen-binding fragment having multiple antigen-binding sites. As such, the terms “bivalent”, “tetravalent”, and “hexavalent” denote the presence of two binding sites, four binding sites, and six binding sites, respectively, in an antigen-binding molecule. In some embodiments, the antibody or antigen-binding fragment thereof is bivalent.

As used herein, a “bispecific” antibody refers to an artificial antibody which has fragments derived from two different monoclonal antibodies and is capable of binding to two different epitopes. The two epitopes may present on the same antigen, or they may present on two different antigens.

In certain embodiments, an “scFv dimer” is a bivalent diabody or bispecific scFv (BsFv) comprising V_{H}-V_{L} (linked by a peptide linker) dimerized with another V_{H}-V_{L} moiety such that V_{H}'s of one moiety coordinate with the V_{L}'s of the other moiety and form two binding sites which can target the same antigens (or epitopes) or different antigens (or epitopes). In other embodiments, an “scFv dimer” is a bispecific diabody comprising V_{H1}-V_{L2} (linked by a peptide linker) associated with V_{L1}-V_{H2} (also linked by a peptide linker) such that V_{H1} and V_{L1} coordinate and V_{H2} and V_{L2} coordinate, and each coordinated pair has a different antigen specificity.

A “dsFv” refers to a disulfide-stabilized Fv fragment that the linkage between the variable region of a single light chain and the variable region of a single heavy chain is a disulfide bond. In some embodiments, a “(dsFv)₂” or “(dsFv-dsFv′)” comprises three peptide chains: two V_{H} moieties linked by a peptide linker (e.g., a long flexible linker) and bound to two V_{L} moieties, respectively, via disulfide bridges. In some embodiments, dsFv-dsFv′ is bispecific in which each disulfide paired heavy and light chain has a different antigen specificity.

The term “chimeric” as used herein, means an antibody or antigen-binding fragment, having a portion of heavy and/or light chain derived from one species, and the rest of the heavy and/or light chain derived from a different species. In an illustrative example, a chimeric antibody may comprise a constant region derived from human and a variable region from a non-human animal, such as from mouse. In some embodiments, the non-human animal is a mammal, for example, a mouse, a rat, a rabbit, a goat, a sheep, a guinea pig, or a hamster.

The term “affinity” as used herein refers to the strength of non-covalent interaction between an immunoglobulin molecule (i.e., antibody) or fragment thereof and an antigen.

The term “specific binding” or “specifically binds” as used herein refers to a non-random binding reaction between two molecules, such as for example between an antibody and an antigen. Specific binding can be characterized in binding affinity, for example, represented by K_{D} value, i.e., the ratio of dissociation rate to association rate (k_{off}/kₒₙ) when the binding between the antigen and antigen-binding molecule reaches equilibrium. K_{D} may be determined by using any conventional method known in the art, including but are not limited to, surface plasmon resonance method, microscale thermophoresis method, HPLC-MS method and flow cytometry (such as FACS) method. A K_{D} value of ≤10⁻⁶ M (e.g. ≤5×10⁻⁷ M, ≤2×10⁻⁷ M, ≤10⁻⁷ M, ≤5×10⁻⁸ M, ≤2×10⁻⁸ M, ≤10⁻⁸ M, ≤5×10⁻⁹ M, ≤4×10⁻⁹M, ≤3×10⁻⁹M, ≤2×10⁻⁹ M, or ≤10⁻⁹ M) can indicate specific binding between an antibody or antigen binding fragments thereof and targeted cell-surface component or surface component of the nanoparticle.

The term “amino acid” as used herein refers to an organic compound containing amine (—NH₂) and carboxyl (—COOH) functional groups, along with a side chain specific to each amino acid. The names of amino acids are also represented as standard single letter or three-letter codes in the present disclosure, which are summarized as follows. Alanine is abbreviated as Ala (A), Arginine as Arg (R), Asparagine as Asn (N), and Aspartic acid as Asp (D). Cysteine is abbreviated as Cys (C), Glutamine as Gln (Q), Glutamic acid as Glu (E), and Glycine as Gly (G). Histidine is His (H), Isoleucine is Ile (I), and Leucine is Leu (L). Lysine is abbreviated as Lys (K), Methionine as Met (M), Phenylalanine as Phe (F), Proline as Pro (P), Serine as Ser (S), Threonine as Thr (T), Tryptophan as Trp (W), Tyrosine as Tyr (Y), and Valine as Val (V).

The term “covalent connection” as used herein refers to a chemical bond where two atoms share electrons with each other, creating a stable connection between the two atoms.

The term “non-covalent connection” as used herein refers to an interaction between two atoms or molecules that doesn't involve the sharing of electrons of the formation or breaking of chemical bonds. Non-covalent connections include without limitation electrostatic interactions, hydrogen bonds, π-effects, van der Waals forces, and hydrophobic effects. Non-covalent connections are involved in many biological processes in which large molecules bind specifically but transiently to one another, such as antibody-antigen binding, ligand-receptor binding.

The term “homologous” as used herein refers to nucleic acid sequences (or its complementary strand) or amino acid sequences that have sequence identity of at least 60% (e.g., at least 65%, 70%, 75%, 80%, 85%, 88%, 90%, 91%, 92%, 93%, 94%, 95%, 96%, 97%, 98%, 99%) to another sequence when optimally aligned.

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). In other words, percent (%) sequence identity of an amino acid sequence (or nucleic acid sequence) can be calculated by dividing the number of amino acid residues (or bases) that are identical relative to the reference sequence to which it is being compared by the total number of the amino acid residues (or bases) in the candidate sequence or in the reference sequence, whichever is shorter. Conservative substitution of the amino acid residues may or may not be considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al., J. Mol. Biol., 215:403-410 (1990); Stephen F. et al., Nucleic Acids Res., 25:3389-3402 (1997)), ClustalW2 (available on the website of European Bioinformatics Institute, see also, Higgins D. G. et al., Methods in Enzymology, 266:383-402 (1996); Larkin M. A. et al., Bioinformatics (Oxford, England), 23(21): 2947-8 (2007)), and ALIGN or Megalign (DNASTAR) software. A person skilled in the art may use the default parameters provided by the tool or may customize the parameters as appropriate for the alignment, such as for example, by selecting a suitable algorithm.

An “isolated” substance has been altered by the hand of man from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide is “isolated” if it has been sufficiently separated from the coexisting materials of its natural state so as to exist in a substantially pure state. An “isolated nucleic acid sequence” refers to the sequence of an isolated nucleic acid molecule. In certain embodiments, an “isolated antibody or an antigen-binding fragment thereof” refers to the antibody or antigen-binding fragments thereof having a purity of at least 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99% as determined by electrophoretic methods (such as SDS-PAGE, isoelectric focusing, capillary electrophoresis), or chromatographic methods (such as ion exchange chromatography or reverse phase HPLC).

The term “vector” as used herein refers to a vehicle into which a genetic element may be operably inserted so as to bring about the expression of that genetic element, such as to produce the protein, RNA or DNA encoded by the genetic element, or to replicate the genetic element. A vector may be used to transform, transduce, or transfect a host cell so as to bring about expression of the genetic element it carries within the host cell. Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating. A vector can be an expression vector or a cloning vector. The present disclosure provides vectors (e.g., expression vectors) containing the nucleic acid sequence provided herein encoding the antibody or an antigen-binding fragment thereof, at least one promoter (e.g., SV40, CMV, EF-1α) operably linked to the nucleic acid sequence, and at least one selection marker.

The phrase “host cell” as used herein refers to a cell into which an exogenous polynucleotide and/or a vector can be or has been introduced.

The term “subject” includes human and non-human animals. Non-human animals include all vertebrates, e.g., mammals and non-mammals, such as non-human primates, mice, rats, cats, rabbits, sheep, dogs, cows, chickens, amphibians, and reptiles. Except when noted, the terms “patient” or “subject” are used herein interchangeably.

“Treating” or “treatment” of a disease, disorder or condition as used herein includes preventing or alleviating a disease, disorder or condition, slowing the onset or rate of development of a disease, disorder or condition, reducing the risk of developing a disease, disorder or condition, preventing or delaying the development of symptoms associated with a disease, disorder or condition, reducing or ending symptoms associated with a disease, disorder or condition, generating a complete or partial regression of a disease, disorder or condition, curing a disease, disorder or condition, or some combination thereof.

The term “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

The term “linker” and “peptide linker” are interchangeably used in the present disclosure and refers to a peptide having natural or synthetic amino acid residues for connecting two polypeptides. For example, the peptide linker may be used to connect the VH and the VL to form the single chain variable fragment (e.g., scFv); or to connect the scFv to the full-length antibody to form a BsAb of the present disclosure. Preferably, the linker is a peptide having at least 2 amino acid residues in length, such as 2 to 200 amino acid residues in length, more preferably 2 to 100 amino acid residues in length. The linker within scFv is a peptide of at least 5 amino acid residues in length, preferably 15 to 20 amino acid residues in length. In some embodiments, the peptide linker includes a hydrophilic amino acid; In some embodiments, the peptide linker includes amino acid glycine (G) and/or serine (S), In some embodiments, the peptide linker includes a sequence of (GS)n, (GGS)n, (GSGGS)n, or (GnS)m, wherein each of n and m is 1 to 10. In one example, the linker comprises a sequence of (GnS)m, with G=glycine, S=serine, n is a number of 1, 2, 3 or 4, and m is 1, 2 or 3. Preferably, the linker comprises a sequence of (G₄S), such as (G₄S)₃, (G₄S)₄; or comprises a sequence of (G₃S) and (G₃S₂). In one example, the linker having a sequence of GGGGS (SEQ ID NO:76), GGGGSGGGGSGGGGS (SEQ ID NO:77), or GGGGSGGGGSGGGGS (SEQ ID NO:78).

The term “covalently binding” and “covalently connection” can be used interchangeably in the present disclosure. Covalently binding refers to the type of chemical interaction where two atoms share one or more pairs of electrons, resulting in the formation of a stable chemical bond. This bond involves the overlap of electron orbitals between atoms, leading to a molecule or molecular complex in which the components are held together through strong, permanent linkages. Covalent bonds are generally stronger and more difficult to break compared to non-covalent interactions.

The term “non-covalently binding” and “non-covalently connection” can be used interchangeably in the present disclosure. Non-covalently binding refers to interactions between molecules or molecular components that do not involve the sharing of electrons. Instead, these interactions are mediated by weaker forces such as hydrogen bonds, van der Waals forces, ionic interactions, or hydrophobic effects. Non-covalent binding is generally reversible and plays a critical role in many biological processes, such as enzyme-substrate interactions, protein-ligand binding, and molecular recognition.

### 2. Overview

The present disclosure in one aspect provides a nanoparticle complex, wherein a lipid nanoparticle non-covalently complexed with a bifunctional binder. The bifunctional binder comprises a self-binder and a target-binder. The self-binder and target-binder may be covalently or noncovalently connected to one another, and the self-binder is noncovalent connected with the nanoparticle, and the target-binder is not connected to the nanoparticle and is able to bind to a target receptor.

Covalently-modified LNPs exist but are difficult to manufacture. The present invention would make manufacturing and scale-up of targeted nanoparticles significantly easier because it would not require an additional chemistry step post-LNP formulation. During manufacture, bifunctional noncovalent binders can be kept separate from the nanoparticle before and during administration, offering additional manufacturing flexibility as conjugated nanoparticles are difficult to keep intact through lyophilization and freezing.

Also, non-covalent binder association in the nanoparticle complex disclosed herein opens the possibility of enhancing endosomal escape compared to a covalently associated binder, as binder stability and antigen binding can be pH-dependent and disulfide reductases are present in endosomes.

In addition, in the nanoparticle complex disclosed herein, associating a nanoparticle with non-covalent binders reduces or substantially eliminates opsonization (e.g., reduces the accelerated blood clearance (ABC)) of the nanoparticle by serum proteins including the endogenous anti-PEG antibodies, which will not be able to substantially bind to the nanoparticle as it may be competing to bind the same target (e.g., PEG) which is already substantially bound by the self-targeting bifunctional binder.

In one embodiment, the inventors finds that, as the methoxy group of PEG is located on the outermost surface of a nanoparticle, an antibody specifically recognizes the methoxy group of PEG can effectively prevent the nanoparticle being recognized by endogenous antibodies, thereby reducing accelerated blood clearance (ABC) and preventing the generation of endogenous antibodies triggered by the methoxy group. This mechanism helps to enhance the stability and therapeutic efficacy of PEGylated drugs in vivo.

Another surprising advantage of the nanoparticle complex provided herein is that it can substantially avoids uptake by hepatocytes, substantially contacts cells displaying the targeted cell-surface component of the target cell (i.e., other than hepatocytes), and substantially reduces the accelerated blood clearance (ABC) of the nanoparticle complex.

### 3. Nanoparticle Complex

In one aspect, the present disclosure provides a nanoparticle complex, comprising: a lipid nanoparticle comprising a surface component; a bifunctional binder; and a cargo carried by the lipid nanoparticle. It is understood that the nanoparticle complex can be formed by mixing the lipid nanoparticle, the bifunctional binder, and the cargo when needed, e.g., before making a pharmaceutical composition for administrating into a subject. The lipid nanoparticle, the bifunctional binder, and the cargo can be packaged or stored separately before mixing. Therefore, the present disclosure in another aspect provides a composition comprising the lipid nanoparticle and the bifunctional binder, wherein the lipid nanoparticle and the bifunctional binder may be packaged separately.

### Bifunctional Binder

In another aspect, the present disclosure provides a bifunctional binder comprising a self-binder moiety non-covalently binding to a surface component of a nanoparticle, and a target-binder moiety capable of binding to a targeted cell-surface component of a target cell.

### Self-Binder Moiety

As used herein, the term “self-binder moiety” refers to a moiety of the bifunctional binder in the nanoparticle complex that is capable of non-covalently binding to the lipid nanoparticle (e.g., to a surface component of the lipid nanoparticle) in the nanoparticle complex.

In some embodiments, the self-binder moiety can be non-covalently bind to any one of the components of the lipid nanoparticle, including an ionizable lipid, a helper lipid, a PEG-modified lipid, or a biotin-modified lipoid (such as a biotin-modified cholesterol) comprised therein. In certain embodiments, the self-binder moiety is capable of specifically and non-covalently binding to the PEG-modified lipid. In certain embodiments, the self-binder moiety is capable of specifically and non-covalently binding to the PEG. In certain embodiments, the self-binder moiety is capable of specifically and non-covalently binding to a molecule conjugated to the ionizable lipid, the helper lipid, the PEG-modified lipid, cholesterol-based lipid or the biotin-modified lipoid (such as a biotin-modified cholesterol). In some embodiments, the molecule conjugated to the ionizable lipid, the helper lipid, the PEG-modified lipid, or the biotin-modified cholesterol.

In some embodiments, the self-binder moiety is a first antigen binding fragment capable of specifically binding to the surface component of the lipid nanoparticle (e.g., the PEG-modified lipid). In some embodiments, the first antigen fragment comprises the variable regions of an antibody (e.g., an anti-PEG antibody), which is derived from an IgG, an IgA, an IgM, an IgE or an IgD.

In certain embodiments, the first antigen binding fragment is derived from a known antibody in the art. For example, a first antigen binding fragment that specifically binds to PEG may be derived from anti-PEG mouse monoclonal antibody (clones 6.3 (Millipore), 3.3 (Creative Biolabs), AGP3 (Millipore), AGP4 (Millipore), rAGP6 (Millipore), 15-2b (Millipore), EPR21993-154 (Abcam), 09F02 (Abcam), 5E10E9 (Thermo Fisher Scientific), ANPEG-1 (ANP Technologies), HL1020 (Thermo Fisher Scientific)), anti-PEG mouse or rabbit polyclonal antibody, anti-PEG rabbit monoclonal antibody (clones EPR21993-154 (Abcam), PEG-B-47 (Abcam), RM105 (Thermo Fisher Scientific), HL1020 (Gene Tex)) or anti-PEG rat monoclonal antibody (clone 26A04, Abcam). In some embodiments, the first antigen binding fragment may be derived from an antibody generated from polyclonal or monoclonal methods known in the art. For example, a first antigen binding fragment that specifically binds to PEG may be derived from a novel anti-PEG antibody disclosed elsewhere herein.

Various types of antigen-binding fragments are known in the art and can be developed based on the antibodies (e.g., an anti-PEG antibody) provided herein, including for example, the exemplary antibodies whose CDRs or VH/VL regions are derived from the parent antibody (e.g., an anti-PEG antibody), and their different variants (such as affinity variants, glycosylation variants, Fc variants, cysteine-engineered variants and so on). In certain embodiments, the first antigen-binding fragment provided herein is a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFab, an scFv dimer (bivalent diabody), a multispecific antibody, a camelized single domain antibody, a nanobody, a domain antibody, or a bivalent domain antibody.

Various techniques can be used for the production of such antigen-binding fragments. Illustrative methods include, enzymatic digestion of intact antibodies (see, e.g. Morimoto et al., Journal of Biochemical and Biophysical Methods 24:107-117 (1992); and Brennan et al., Science, 229:81 (1985)), recombinant expression by host cells such as E. Coli (e.g. for Fab, Fv and ScFv antibody fragments), screening from a phage display library as discussed above (e.g. for ScFv), and chemical coupling of two Fab′-SH fragments to form F(ab′)₂ fragments (Carter et al., Bio/Technology 10:163-167 (1992)). Other techniques to produce antibody fragments will be apparent to a person skilled in the art.

In certain embodiments, the antigen-binding fragment is an scFv. Generation of scFv is described in, for example, WO 93/16185; U.S. Pat. Nos. 5,571,894; and 5,587,458. ScFv may be fused to an effector protein at either the amino or the carboxyl terminus to provide for a fusion protein (see, for example, Antibody Engineering, ed. Borrebaeck).

In certain embodiments, the self-binders provided herein are bivalent, tetravalent, hexavalent, or multivalent. Any molecule being more than bivalent is considered multivalent, encompassing for example, trivalent, tetravalent, hexavalent, and so on. A bivalent molecule can be monospecific if the two binding sites are both specific for binding to the same antigen or the same epitope. This, in certain embodiments, provides for stronger binding to the antigen or the epitope than a monovalent counterpart. Similarly, a multivalent molecule may also be monospecific. In certain embodiments, in a bivalent or multivalent antigen-binding moiety, the first valent of binding site and the second valent of binding site are structurally identical (i.e., having the same sequences), or structurally different (i.e., having different sequences albeit with the same specificity). A bivalent can also be bispecific, if the two binding sites are specific for different antigens or epitopes. This also applies to multivalent molecules. For example, a trivalent molecule can be bispecific when two binding sites are monospecific for a first antigen (or epitope) and the third binding site is specific for a second antigen (or epitope).

### Anti-PEG Antibody

In some embodiments, the self-binder moiety is a first antigen binding fragment that specifically binds to PEG derived from a novel anti-PEG antibody as disclosed herein. Therefore, the present disclosure in another aspect provides an anti-PEG antibody or an antigen-binding fragment thereof.

In some embodiments, the anti-PEG antibody or an antigen-binding fragment thereof comprising: a heavy chain (HC) variable region (VH) comprising HC-CDR1, HC-CDR2 and HC-CDR3; and a light chain (LC) variable region (VL) comprising LC-CDR1, LC-CDR2 and LC-CDR3, wherein
(a) the HC-CDR1, HC-CDR2 and HC-CDR3 sequences are those contained in a VH sequence of SEQ ID NO: 7, and the LC-CDR1, LC-CDR2 and LC-CDR3 sequences are those contained in a VL sequence of SEQ ID NO: 8, or
(b) the HC-CDR1, HC-CDR2 and HC-CDR3 sequences are those contained in a VH sequence of SEQ ID NO: 15, and the LC-CDR1, LC-CDR2 and LC-CDR3 sequences are those contained in a VL sequence of SEQ ID NO: 16.

In some embodiments, the HC-CDR1, HC-CDR2, HC-CDR3, LC-CDR1, LC-CDR2, and LC-CDR3 sequences of the anti-PEG antibody or an antigen-binding fragment thereof are defined by the Kabat numbering system, the IMGT numbering system, the Chothia numbering system, the Contact numbering system, or the AbM numbering system.

Unless clearly indicated to the contrary, the numbering system used in the embodiments of the present disclosure is IMGT numbering system.

In some embodiments, the HC-CDR1, HC-CDR2 and HC-CDR3 sequences of the anti-PEG antibody or an antigen-binding fragment thereof are set forth in SEQ ID NOs 1-3 respectively; and the LC-CDR1, LC-CDR2 and LC-CDR3 sequences of the anti-PEG antibody or an antigen-binding fragment thereof are set forth in SEQ ID NOs 4-6 respectively.

In some embodiments, the HC-CDR1, HC-CDR2 and HC-CDR3 sequences of the anti-PEG antibody or an antigen-binding fragment thereof are set forth in SEQ ID NOs 9-11 respectively; and the LC-CDR1, LC-CDR2 and LC-CDR3 sequences of the anti-PEG antibody or an antigen-binding fragment thereof are set forth in SEQ ID NOs 12-14 respectively.

In some embodiments, the VH of the anti-PEG antibody or an antigen-binding fragment thereof comprises a sequence having at least 80%, 85%, 90%, or 95% identity to SEQ ID NO: 7, and the VL of the anti-PEG antibody or an antigen-binding fragment thereof comprises a sequence having at least 80%, 90%, or 95% identity to SEQ ID NO: 8. In some embodiments, the VH of the anti-PEG antibody or an antigen-binding fragment thereof comprises a sequence of SEQ ID NO: 7, and the VL of the anti-PEG antibody or an antigen-binding fragment thereof comprises a sequence of SEQ ID NO: 8.

In some embodiments, the VH of the anti-PEG antibody or an antigen-binding fragment thereof comprises a sequence having at least 80%, 85%, 90%, or 95% identity to SEQ ID NO: 15, and the VL of the anti-PEG antibody or an antigen-binding fragment thereof comprises a sequence having at least 80%, 85%, 90%, or 95% identity to SEQ ID NO: 16. In some embodiments, the VH of the anti-PEG antibody or an antigen-binding fragment thereof comprises a sequence of SEQ ID NO: 15, and the VL of the anti-PEG antibody or an antigen-binding fragment thereof comprises a sequence of SEQ ID NO: 16.

In another aspect, the present disclosure provides an isolated polynucleotide encoding the anti-PEG antibody or antigen-binding fragment thereof described herein.

In another aspect, the present disclosure provides a vector comprising the isolated polynucleotide described herein.

In another aspect, the present disclosure provides a host cell comprising the vector described herein. In certain embodiments, the host cell is a mammalian cell, e.g., a CHO cell.

In another aspect, the present disclosure provides a hybridoma encoding or producing the anti-PEG antibody as provided herein.

In another aspect, the present disclosure provides a method of expressing the anti-PEG antibody or antigen-binding fragment thereof described herein. In some embodiments, the method comprises culturing the host cell described herein under the condition at which the vector described herein is expressed.

### Target-Binder Moiety

As used herein, the term “target-binder moiety” refers to a moiety of the bifunctional binder in the nanoparticle complex that can target a specific cell type, e.g., by specifically recognizing and binding to the targeted cell-surface component of the target cell.

In some embodiments, the target cell includes without limitation an antigen presenting cell, a monocyte, a neutrophil, a macrophage, a dendritic cell, a mast cell, a T cell, a natural killer cell, a Kupffer cell, a B cell, or a tumor cell. In some embodiments, the target cell is a T cell selected from a CD3+ T cell, a CD4+ T cell, a CD8+ T cell, a T regulatory cell, a cytotoxic T cell, and a helper T cell.

In some embodiments, the cell-surface component of the target cell is a protein (including a receptor or a non-receptor protein), a glycoRNA, a lipid, a lipid raft, or the like.

In certain embodiments, the cell-surface component of the target cell is a receptor. In some embodiments, the receptor is an Fc receptor, FcgR1, FcgR2, FCgr3, FcgR4, FceR1, FceR2, FcaR1, FcuR, FcdR, or an isoform thereof, or C1qR or FcRn, or the like. In some embodiments, the receptor is CD2, CD3, CD4, CD5, CD7, CD8, CD25, CD45RA, CD45RO, CTLA4, PD-L1 or the like. In some embodiments, the receptor is an adrenergic receptor, an olfactory receptor, a receptor tyrosine kinase, an epidermal growth factor receptor, an insulin receptor, a fibroblast growth factor receptor, a neurotrophin receptor, an ephrin receptor, an NMDA receptor, a toll-like receptor, or a T-cell receptor.

In some embodiments, the tumor cell is selected from brain tumor cells, lung cancer cells, squamous carcinoma cells, bladder carcinoma cells, gastric cancer cells, ovarian cancer cells, peritoneal carcinoma cells, pancreatic carcinoma cells, breast cancer cells, head and neck cancer cells, cervical cancer cells, endometrial cancer cells, rectal cancer cells, liver cancer cells, renal carcinoma cells, esophageal adenocarcinoma cells, esophageal squamous cancer cells, prostatic cancer cells, female reproductive duct cancer cells, cancer in situ cells, lymphoma cells, neurofibromas cells, thyroid carcinoma cells, osteocarcinoma cells, skin cancer cells, brain cancer cells, colon cancer cells, testiculus cancer cells, gastrointestinal stromal tumor cells, prostate neoplasms cells, mast cell tumor cells, multiple myeloma cells, melanoma cells, glioma cells, sarcoma cells, esophageal cancer cells, acute leukemia cells, malignant neoplasm cells of female genital organs, myeloma cells, teratoma cells, neuroblastoma cells, rhabdomyosarcoma cells, meningioma cells, colorectal cancer cells, cholangiocarcinoma cells, medulloepithelioma cells, anaplastic astrocytoma cells, malignant mesothelioma cells, malignant peripheral nerve sheath tumor cells, undifferentiated (embryonal) sarcoma cells, myelofibrosis cells, liposarcoma cells, dedifferentiated liposarcoma cells, soft-tissue sarcoma cells, ewing sarcoma cells, angiosarcoma cells, epithelioid sarcoma cells, fibrosarcoma cells, leiomyosarcoma cells, synovial sarcoma cells or myxofibrosarcoma cells.

In some embodiments, the cell-surface component of the tumor cell is a tumor-associated antigen (TAA).

In some embodiments, the TAA is selected from the group consisting of: HER2, GUCY2C, MSLN, 0772P, 5T4, ACTA2, ADGRE1A, AGS-16, AKR1C1, AKR1C2, ANGPTL4, ApoE, ASLG659, ASLG659, AIF1, BMPR1B, BNIP3, Brevican, BAFF-R, BCMA, c-Met, CADM1, CA6, C1QA, C1QB, CCL5, CCR4, CCR5, CD1lb, CD1lc, CD138, CD19, CD20, CD21, CD22, CD223, CD30, CD33, CD37, CD40, CD45(PTPRC), CD49D(ITGA4), CD56, CD66e, CD70, CD72, CD74, CD79a, CD79b, CD80, CDCP1, CDH11, Claudin18.2, COL6A3, COL7A1, CRIPTO, CSF1R, CTGF, CTSD, CTSS, CXCL10, CXCL11, CXCR4, CXCR5, DDIT4, DLL3, DLL4, DR5, E16, EFNA4, EGFR, EGFRvIII, EGLN, EGLN3, EMR2, ENPP1, ENPP3, Endothelinreceoptor, EphB2R, EpCAM, ETBR, FcRH1, FcRH2, FGF2, FGFR1, FGFR2, FGFR3, FGFR4, Folatereceptor 1, GEDA, GPC3, GPNMB, Guanylylcyclase C, GZMB HER3, HLA-DOB, HMOX1, KRT33A, KISS1R, IFNG, IGFBP3, IFI6, IGF-1R, IL-6, IL10RA1, IL20R α, IRTA2, LOX, LRRC15, LY64, LY6E, LY86, LYPD3, LUM, MCPT8, MDP, Mesothelin, MFI2, MMP9, MMP10, MMP14, MMP16, MPF, MS4A7, MSG783, Mucin 1, Mucin16, Napi2b, Napi3b, NCA, Nectin 4, NOG, P2X5, P2X7, pCAD, PGF, PIK3AP1, PIK3CD, PD-L1, PDGFRA, PDK1, PDK4, PFKFB33, PGK1, PLOD2, PSCA, PSCAhlg, RNF43, ROR1, Sema 5b, SERPINEl, SLC39A6, SLTRK6, STEAP1, STEAP2, Sodiumphosphate cotransporter2B, STAT1, STAT3, STC2, TACSTD2, TENB2, Tissue factor, TCF4, TENB2, TGF, TGFB1, TGFB2, TGFBR1, TNFR2, TNFRSF21, TNFSF9, TrpM4, Trop-2, Trophoblast glycoprotein Tyro7, PSMA, VEGFA, VEGFR2, WNT5A, UPK1B, E-cadherin, P-cadherin, integrin α5β6 and integrin α4β7.

In some embodiments, the target-binder moiety is an Fc or a fragment thereof of an IgG, an IgA, an IgM, an IgE or an IgD. In some embodiments, the target-binder moiety is derived from an Fc and comprises a CH2-CH3 fragment, a CH2-CH3-CH4 fragment, a CH3-CH4 fragment, or the like.

In some embodiments, the target-binder moiety is a second antigen binding fragment capable of specifically binding to the cell-surface component of the target cell. In some embodiments, the second antigen fragment comprises the variable regions of an antibody, which is derived from an IgG, an IgA, an IgM, an IgE or an IgD.

In certain embodiments, the second antigen-binding fragment provided herein is a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFab, an scFv dimer (bivalent diabody), a multispecific antibody, a camelized single domain antibody, a nanobody, a domain antibody, and a bivalent domain antibody.

### Structure of the Bifunctional Binder

In some embodiments, the self-binder moiety is covalently linked to the target-binder moiety. In some embodiments, the self-binder moiety is linked to the target-binder moiety via a linker.

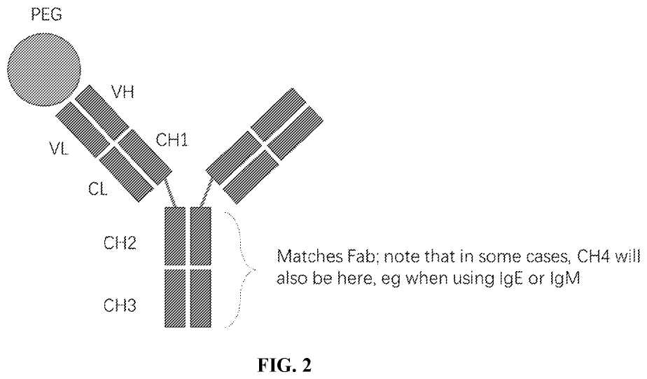

One exemplary structure of the bifunctional binder provided herein is shown in FIG. 2. In this embodiment, the self-binder moiety (i.e., Fab region) and the target-binder moiety (e.g., CH2-CH3 region, which may also comprise a CH4 region) are derived from the same antibody subtype. Such a bifunctional binder can naturally facilitate antigen uptake into APCs, as has been shown for IgM in accelerated blood clearance of LNPs.

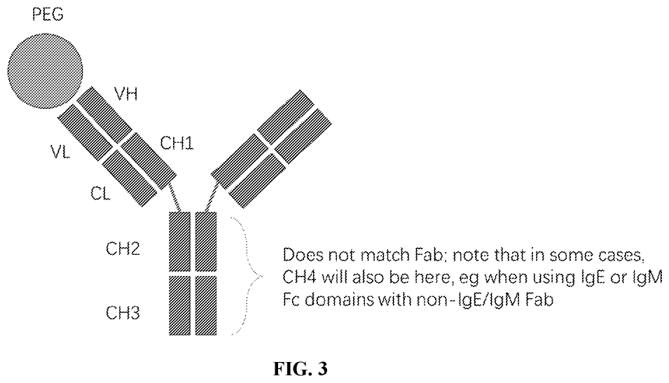

One exemplary structure of the bifunctional binder provided herein is shown in FIG. 3. In this embodiment, the self-binder moiety (i.e., Fab region) and the target-binder moiety (e.g., CH2-CH3 region, which may also comprise a CH4 region) are derived from different antibody subtypes. Such a bifunctional binder allows the nanoparticle complex to retarget cell types that express different types of Fc receptor, or a combination of Fc receptors.

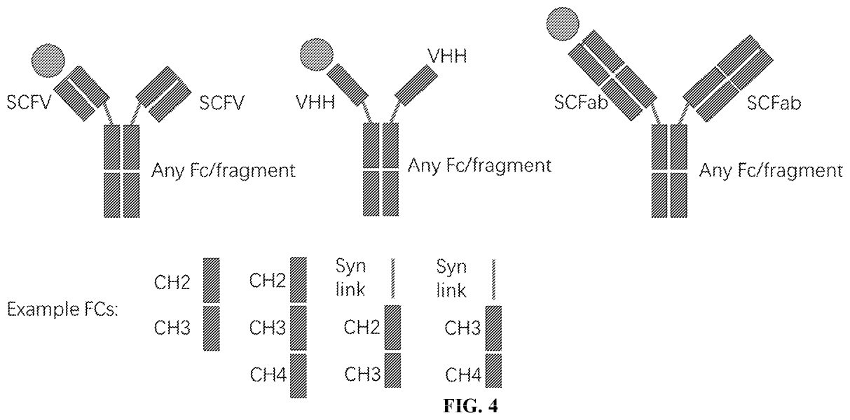

Some exemplary structures of the bifunctional binder provided herein are shown in FIG. 4. The self-binder moiety may be an scFv, a VHH, an scFab or the like, while the target-binder moiety is an Fc or fragments thereof. The self-binder moiety and the target-binder moiety are connected via a linker, e.g., a synthetic linker. Such structures are with smaller self-binders, which may allow higher density on LNP surface.

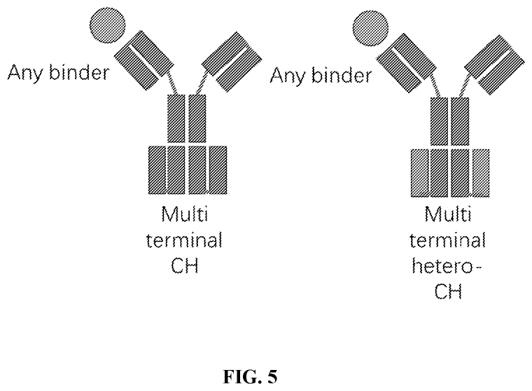

One exemplary structure of the bifunctional binder provided herein is shown in FIG. 5. The target-binder moiety of the bifunctional binder is derived from an Fc or fragments thereof, and has multiple terminal CHs. As used herein, the term “terminal CH” refers to the CH region at the C terminal of the Fc region in an antibody, such as CH3 for an IgG antibody, or CH4 for an IgM antibody). Such bifunctional binder structure may improve target cell uptake when using more than one subtype of Fcs as targeting binders, wherein hetero-CH may allow for targeting of multiple receptors/cell types simultaneously.

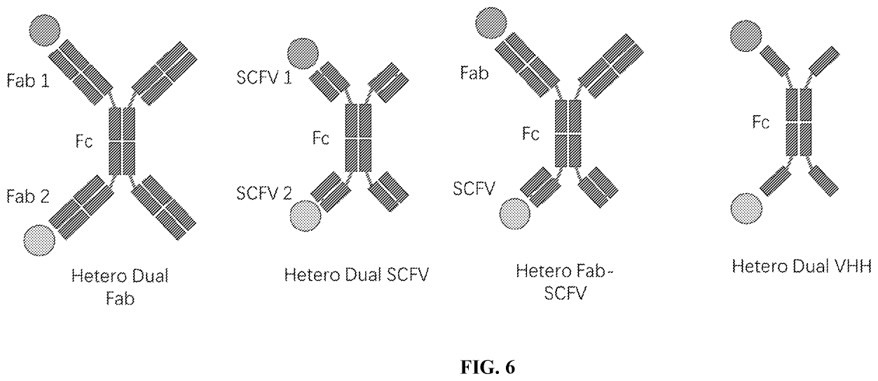

Some exemplary structures of the bifunctional binder provided herein are shown in FIG. 6. This kind of format does not use Fc or the fragments thereof as the target-binder moiety, but as the linker between the self-binder moiety and the target-binder moiety. In other words, Fc is solely used for dimerization and does not have any target-binding effect. Both the self-binder moiety and the target-binder moiety are derived from variable regions of antibodies, which are namely a first antigen-binding fragment and a second antigen-binding fragment, respectively. Such bifunctional binder structures enable targeting of the nanoparticle complexes to non-Fc receptor expressing cellular targets.

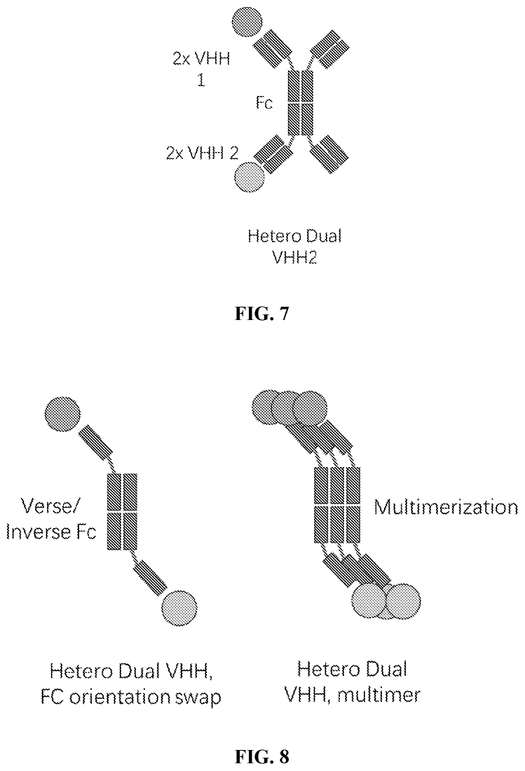

One exemplary structure of the bifunctional binder provided herein is shown in FIG. 7. Similar to those structures shown in FIG. 6, Fc regions herein are solely used for dimerization and does not have any target-binding effect, and both the self-binder moiety and the target-binder moiety are derived from variable regions of antibodies, which are namely a first antigen-binding fragment and a second antigen-binding fragment, respectively. The difference from FIG. 6 is that the self-binder moiety or the target-binder moiety may comprise more than one antigen-binding fragment types, including but not limited to Fab, SCFV, VHH, etc. For example, the self-binder moiety may comprise two arms, each comprising two VHHs (thus total of four VHHs). Such multiple binders increase avidity and can improve target engagement and LNP surface engagement.

Other exemplary structures of the bifunctional binder provided herein are shown in FIG. 8. Referring to the left panel of FIG. 8, the bifunctional binder comprises a pair of verse/inverse Fc, each connected to a VHH. The two VHHs are self-binder moiety and target-binder moiety, respectively. Referring to the right panel of FIG. 8, the bifunctional binder comprises multimerized Fc domains, each connected to a VHH, which can be either a self-binder moiety or target-binder moiety. In the right panel of FIG. 8, the bifunctional binder has three self-binder moieties and three target-binder moieties. The formats can be generated by modulating the Fc domain into other multimerization domains. In such bifunctional structures, multimerization beyond dimers can increase avidity.

### Lipid Nanoparticle

In some embodiments, the lipid nanoparticle in the nanoparticle complex provided herein comprises an ionizable lipid, a helper lipid, a PEG-modified lipid and a cholesterol-based lipid.

In some embodiments, the lipid nanoparticle in the nanoparticle complex provided herein also comprises biotin-modified lipoid, or other components modified lipid or lipoid.

In some embodiments, the lipid nanoparticle comprises:40-50 mol % of the ionizable lipid, 30-45 mol % of the cholesterol-based lipid, 5-15 mol % of the helper lipid, 1-5 mol % of the PEG-modified lipid.

In some embodiments, the lipid nanoparticle comprises:40-50 mol % of ALC-0315 or SM102 or Lipid5; 30-45 mol % of the cholesterol-based lipid; 5-15 mol % of DOPE or DSPC; 1-5 mol % of the PEG-modified lipid (e.g DMG-PEG2000, DSPE-PEG2000, or combination thereof).

In some embodiments, the lipid nanoparticle comprises: ALC-0315, cholesterol, DOPE and DMG-PEG2000.

In some embodiments, the lipid nanoparticle comprises: ALC-0315, cholesterol, DSPC, DMG-PEG2000 and DSPE-PEG2000.

In some embodiments, the lipid nanoparticle comprises: SM102, cholesterol, DOPE, DMG-PEG2000, DSPE-PEG2000 and 18:1PG.

In some embodiments, the lipid nanoparticle comprises: Lipid5, cholesterol, DSPC, DMG-PEG2000, DSPE-PEG2000 and 18:1PG.

In some embodiments, the lipid nanoparticle comprises: SM102, cholesterol, DSPC and DMG-PEG2000.

### Ionizable Lipids

As used herein, the term “ionizable lipid” refers to a class of lipid molecules which remain neutral at physiological pH, but are protonated at low pH, making them positively charged, which promotes nucleic acid complexation, endosomal escape and reduces toxicity of lipid nanoparticles.

In some embodiments, the ionizable lipid is a cationic lipid. As used in herein, the term “cationic lipid” refers to any of a number of lipid species that have a net positive charge at a selected pH, such as physiological pH. Some cationic lipids, in particular, those known as titratable or pH-titratable cationic lipids are particularly effective in delivering mRNA. Several cationic (e.g., titratable) lipids have been described in the literature, many of which are commercially available. In some embodiments, the cationic lipid is selected from the but not limited to HGT4003 (WO 2012/170889, the teachings of which are incorporated herein by reference in their entirety), HGT5000 ((15Z, 18Z)—N,N-dimethyl-6-(9Z, 12Z)-octadeca-9, 12-dien-1-yl)tetracosa-15,18-dien-1-amine, as described in U.S. 61/617,468), HGT5001 ((15Z, 18Z)—N,N-dimethyl-6-((9Z, 12Z)-octadeca-9, 12-dien-1-yl)tetracosa-4,15,18-trien-1-amine, as described in U.S. 61/617,468), HGT5002 ((15Z,18Z)—N,N-dimethyl-6-((9Z, 12Z)-octadeca-9, 12-dien-1-yl)tetracosa-5, 15, 18-trien-1-amine, as described in U.S. 61/617,468), DOTMA (1,2-di-O-octadecenyl-3-trimethylammonium propane, as described in Feigner et al. (Proc. Nat'l Acad. Sci. 84, 7413 (1987); U.S. Pat. No. 4,897,355), DOGS (5-carboxyspermylglycinedioctadecylamide), DOSPA (2,3-dioleyloxy-N-[2(spermine-carboxamido)ethyl]-N,N-dimethyl-1-propanaminium, as described in Behr et al. Proc. Nat.'l Acad. Sci. 86, 6982 (1989); U.S. Pat. Nos. 5,171,678; 5,334,761), DOTAP (1,2-Dioleoyl-3-Trimethylammonium-Propane), DODAP (1,2-Dioleoyl-3-Dimethylammonium-Propane), DSDMA (1,2-distearyloxy-N,N-dimethyl-3-aminopropane), DODMA (1,2-dioleyloxy-N,N-dimethyl-3-aminopropane), DLinDMA (1,2-dilinoleyloxy-N,N-dimethyl-3-aminopropane, as described in Heyes, J.; Palmer, L.; Bremner, K.; MacLachlan, I. “Cationic lipid saturation influences intracellular delivery of encapsulated nucleic acids” J. Contr. Rel. 2005, 107, 276-287), DLenDMA (1,2-dilinolenyloxy-N,N-dimethyl-3-aminopropane), DODAC (N-dioleyl-N,N-dimethylammonium chloride), DDAB (N,N-distearyl-N,N-dimethylarnrnonium bromide), DMRIE (N-(1,2-dimyristyloxyprop-3-yl)-N,N-dimethyl-N-hydroxyethyl ammonium bromide), CLinDMA (3-dimethylamino-2-(cholest-5-en-3-beta-oxybutan-4-oxy)-1-(cis,cis-9,12-octadecadienoxy)propane), CpLinDMA (2-[5′-(cholest-5-en-3-beta-oxy)-3′-oxapentoxy)-3-dimethy 1-1-(cis,cis-9′, 1-2′-octadecadienoxy)propane), DMOBA (N,N-dimethyl-3,4-dioleyloxybenzylamine), DOcarbDAP (1,2-N,N′-dioleylcarbamyl-3-dimethylaminopropane), DLinDAP (2,3-Dilinoleoyloxy-N,N-dimethylpropylamine), DLincarbDAP (1,2-N,N′-Dilinoleylcarbamyl-3-dimethylaminopropane), DLinCDAP (1,2-Dilinoleoylcarbamyl-3-dimethylaminopropane), DLin-K-XTC2-DMA (2,2-dilinoleyl-4-dimethylaminoethyl-[1,3]-dioxolane), DLin-KC2-DMA (2-(2,2-di((9Z,12Z)-octadeca-9,1 2-dien-1-yl)-1,3-dioxolan-4-yl)-N,N-dimethylethanamine), AA3-DLin (piperazine-1,4-diylbis(ethane-2,1-diyl) (9z,9′z,12z,12′z)-bis(octadeca-9,12-dienoate)), XTC (2,2-Dilinoley 1-4-dimethylaminoethyl-[1,3]-dioxolane), ALNY-100 ((3aR,5s,6aS)—N,N-dimethyl-2,2-di((9Z,12Z)-octadeca-9,12-dienyl)tetrahydro-3aH-cyclopenta[d][1,3]dioxol-5-amine)), NC98-5 (4,7,13-tris(3-oxo-3-(undecylamino)propyl)-N1,N16-diundecyl-4,7,10,13-tetraazahexadecane-1,16-diamide), C12-200 (Love, K. T. et al. “Lipid-like materials for low-dose in vivo gene silencing” PNAS 2010, 107, 1864-1869), MC2, MC3 (((6Z,9Z,28Z,31Z)-heptatriaconta-6,9,28,31-tetraen-19-yl 4-(dimethylamino)butanoate), MC4, ALC-0315 ([(4-hydroxybutyl)azanediyl]di(hexane-6,1-diyl) bis(2-hexyldecanoate)), SM-102 (9-Heptadecanyl 8-{(2-hydroxyethyl)[6-oxo-6-(undecyloxy)hexyl]amino}octanoate), ATX-001 (di((Z)-non-2-en-1-yl) 8,8′-((2-((2-(dimethylamino)ethyl)thio)acetyl)azanediyl)dioctanoate), ATX-100 (4,4′-[[[[3-(dimethylamino)propyl]thio]carbonyl]imino]bis-butanoic acid, 1,1′-bis(1-heptyloctyl) ester), Lipid 2, Lipid 5, 9A1P9 (2-(dioctylamino)ethyl nonyl hydrogen phosphate), OF-Deg-Lin (9,12-Octadecadienoic acid (9Z,12Z)-, 1,1′,1″,1′″-[(3,6-dioxo-2,5-piperazinediyl)bis(4,1-butanediylnitrilodi-2,1-ethanediyl)]ester), 80-016B (N-[3-(dimethylamino)propyl]-N-[3-[2-(dodecyldithio)ethoxy]-3-oxopropyl]-o-alanine, 2-(dodecyldithio)ethyl ester), 93-017S (bis(2-(tetradecylthio)ethyl) 3,3′-((3-(1H-imidazol-1-yl)propyl)azanediyl)dipropionate), 93-O17O (bis(2-(tetradecyloxy)ethyl) 3,3′-((3-(1H-imidazol-1-yl)propyl)azanediyl)dipropionate), 306-012B (tetrakis(2-(octyldisulfaneyl)ethyl) 3,3′,3″,3′″-(((methylazanediyl)bis(propane-3,1-diyl))bis(azanetriyl))tetrapropionate), 113-O16B (tetrakis(2-(dodecyldisulfaneyl)ethyl) 3,3′,3″,3′″-(((methylazanediyl)bis(ethane-2,1-diyl))bis(azanetriyl))tetrapropionate), 306Oi10 (tetrakis(8-methylnonyl) 3,3′,3″,3′″-(((methylazanediyl)bis(propane-3,1-diyl))bis(azanetriyl))tetrapropionate), 113-O12B (tetrakis(2-(octyldisulfaneyl)ethyl) 3,3′,3″,3′″-(((methylazanediyl)bis(ethane-2,1-diyl))bis(azanetriyl))tetrapropionate), cKK-E12 (3,6-bis[4-[bis(2-hydroxydodecyl)amino]butyl]-2,5-piperazinedione), 98N12-5 (N1,N16-didodecyl-4,7,13-tris[3-(dodecylamino)-3-oxopropyl]-4,7,10,13-tetraazahexadecanediamide), OF-02 (3,6-bis[4-[bis[(9Z,12Z)-2-hydroxy-9,12-octadecadien-1-yl]amino]butyl]-2,5-piperazinedione), TLC053 (2-(((4-(dimethylamino)butanoyl)oxy)methyl)-2-((((Z)-tetradec-9-enoyl)oxy)methyl)propane-1,3-diyl (9Z,9′Z)-bis(tetradec-9-enoate)), LPO1 (9Z,12Z-octadecadienoic acid, 3-[4,4-bis(octyloxy)-1-oxobutoxy]-2-[[[[3-(diethylamino)propoxy]carbonyl]oxy]methyl]propyl ester), BAMEA-O16B (bis(2-(dodecyldisulfaneyl)ethyl) 3,3′-((3-methyl-9-oxo-10-oxa-13,14-dithia-3,6-diazahexacosyl)azanediyl)dipropionate), CL1 (5-(dimethylamino)-pentanoic acid, (6Z)-1,2-di-(4Z)-4-decen-1-yl-6-dodecen-1-yl ester), BP Lipid 310, Lipid A9 (bis(2-butyloctyl) 10-(N-(3-(dimethylamino)propyl)nonanamido)nonadecanedioate), L319 (9-[4-(dimethylamino)-1-oxobutoxy]-heptadecanedioic acid, 1,17-di-(2Z)-2-nonen-1-yl ester), and ICE (WO 2011/068810, the teachings of which are incorporated herein by reference in their entirety).

### Helper Lipids Non-Cationic Lipids

In some embodiments, the lipid nanoparticle in the nanoparticle complex provided herein contains one or more helper lipids. As used herein, the terms “helper lipid” and “non-cationic lipid” are used interchangeably, which refer to any neutral, zwitterionic or anionic lipid. As used herein, the phrase “anionic lipid” refers to any of a number of lipid species that carry a net negative charge at a selected pH, such as physiological pH. Non-cationic lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), or a mixture thereof.

In some embodiments, such non-cationic lipids may be used alone, but are preferably used in combination with other excipients, for example, cationic lipids. In some embodiments, the non-cationic lipid may comprise a molar ratio of about 5% to about 90%, or about 10% to about 70% of the total lipid present in a lipid nanoparticle. In some embodiments, a non-cationic lipid is a neutral lipid, i.e., a lipid that does not carry a net charge in the conditions under which the composition is formulated and/or administered. In some embodiments, the percentage of non-cationic lipid in a lipid nanoparticle may be greater than 5%, greater than 10%, greater than 20%, greater than 30%, or greater than 40%.

### Cholesterol-Based Lipid

In some embodiments, the lipid nanoparticle in the nanoparticle complex provided herein contains one or more cholesterol-based lipids. For example, suitable cholesterol-based cationic lipids include, but not limited to, DC-Choi (N,N-dimethyl-N-ethylcarboxamidocholesterol), 1,4-bis(3-N-oleylamino-propyl)piperazine (Gao, et al. Biochem. Biophys. Res. Comm. 179, 280 (1991); Wolf et al. BioTechniques 23, 139 (1997); U.S. Pat. No. 5,744,335), or ICE.

In some embodiments, the cholesterol-based lipid may comprise a molar ration of about 2% to about 50%, or about 5% to about 20% of the total lipid present in a lipid nanoparticle. In some embodiments, The percentage of cholesterol-based lipid in the lipid nanoparticle may be greater than 5, %, 10%, greater than 20%, greater than 30%, or greater than 40%.

### PEG-Modified Lipid

In some embodiments, the lipid nanoparticle in the nanoparticle complex provided herein contains one or more PEG-modified lipids. For example, the use of polyethylene glycol (PEG)-modified phospholipids and derivatized lipids such as derivatized ceramides (PEG-CER), including N-Octanoyl-Sphingosine-l-[Succinyl(Methoxy Polyethylene Glycol)-2000](C8 PEG-2000 ceramide) is also contemplated by the present invention in combination with one or more of the cationic and, in some embodiments, other lipids together which comprise the lipid nanoparticle. Contemplated PEG-modified lipids include, but are not limited to, a polyethylene glycol (PEG) chain covalently attached to a lipid. In some embodiments, the PEG chain is of 100-20000 Da, preferably the PEG chain is of 100, 200, 400, 600, 800, 1000, 2000, 4000, 8000, 10000, 20000 Da. In some embodiments, the alkyl chain is of C₆-C₂₀ length.

In some embodiments, a PEG-modified or PEGylated lipid is PEGylated cholesterol or PEG-2K. The addition of such components may prevent complex aggregation and may also provide a means for increasing circulation lifetime and increasing the delivery of the lipid-nucleic acid composition to the target cell, (Klibanov et al. (1990) FEBS Letters, 268 (1): 235-237), or they may be selected to rapidly exchange out of the formulation in vivo (see U.S. Pat. No. 5,885,613).

In some embodiments, the PEG-modified lipid is selected from the group consisting of PEG-modified phosphatidylethanolamines, PEG-modified phosphatidic acids, PEG-modified ceramides, PEG-modified dialkylamines, PEG-modified diacylglycerols, and PEG-modified dialkylglycerols. For example, a PEG lipid may be PEG-dioleoylgylcerol (PEG-DOG), PEG-dimyristoyl-glycerol (PEG-DMG), PEG-dipalmitoyl-glycerol (PEG-DPG), PEG-dilinoleoyl-glycero-phosphatidyl ethanolamine (PEG-DLPE), PEG-dimyrstoyl-phosphatidylethanolamine (PEG-DMPE), PEG-dipalmitoyl-phosphatidylethanolamine (PEG-DPPE), PEG-distearoylglycerol (PEG-DSG), PEG-diacylglycerol (PEG-DAG, e.g., PEG-DMG, PEG-DPG, and PEG-DSG), PEG-ceramide, PEG-distearoyl-glycero-phosphoglycerol (PEG-DSPG), PEG-dioleoyl-glycero-phosphoethanolamine (PEG-DOPE), 2-[(polyethylene glycol)-2000]-N,N-ditetradecylacetamide, or a PEG-distearoyl-phosphatidylethanolamine (PEG-DSPE) lipid.

In some embodiments, the PEG-modified lipid is a glycol (PEG)-modified phospholipid or ceramide, or a combination thereof. In some embodiments, the PEG-modified lipid is DMG-PEG2000, DSPE-PEG2000 or a combination thereof.

In some embodiments, particularly useful exchangeable lipids are PEG-ceramides having shorter acyl chains (e.g., C₁₄ or C₁₈). The PEG-modified phospholipid and derivatized lipids of the present invention may comprise a molar ratio from about 0% to about 15%, about 0.5% to about 15%, about 1% to about 15%, about 4% to about 10%, or about 2% of the total lipid present in the lipid nanoparticle.

In some embodiments, the PEG-modified lipid is sheddable PEG-modified lipid, non-sheddable PEG-modified lipid, or combinations thereof.

The alkyl chain length of the sheddable PEG-modified lipid is shorter than that of the non-sheddable PEG-modified lipid.

In some embodiments, the PEG-modified lipid comprises the sheddable PEG-modified lipid and the non-sheddable PEG-modified lipid.

In some embodiments, the mole ratio of the sheddable to the non-sheddable PEG-modified lipid is (1-3):(1-3). In some embodiments, the mole ratio of the sheddable to the non-sheddable PEG-modified lipid is (1-3):1. In some embodiments, the mole ratio of the sheddable to the non-sheddable PEG-modified lipid is 1:(1-3).

According to various embodiments, the selection of cationic lipids, non-cationic lipids and/or PEG-modified lipids in the lipid nanoparticle, as well as the relative molar ratio of such lipids to each other, is based upon the characteristics of the selected lipid(s), the nature of the intended target cells, the characteristics of the cargos to be delivered. Additional considerations include, for example, the saturation of the alkyl chain, as well as the size, charge, pH, pKa, fusogenicity and toxicity of the selected lipid(s). Thus, the molar ratios may be adjusted accordingly. In some embodiments, the percentage of PEG-modified lipid in a lipid nanoparticle may be greater than 1%, greater than 2%, greater than 5%, greater than 10%, or greater than 15%.

### Cargo

The nanoparticle complex disclosed herein may be used to delivery any therapeutic agents. The cargo to be delivered may be an organic molecule (e.g., a therapeutic agent, a drug), inorganic molecule, nucleic acid, protein, amino acid, peptide, polypeptide, polynucleotide, targeting agent, isotopically labeled organic or inorganic molecule, vaccine, immunological agent, etc. In certain embodiments of the present invention, the cargo to be delivered may be a mixture of agents.

In some embodiments, the cargo is one or more nucleic acid molecules, nucleosides, nucleotides or combination thereof encapsulated in the lipid nanoparticle. In some embodiments, the nucleic acid molecule, nucleotide, or nucleoside is selected from the group consisting of a circular RNA, a mRNA, a noncoding RNA, a dsRNA, a miRNA, an siRNA or an antisense RNA, a tRNA, an ssDNA, a plasmid DNA, Adenosine, Adenosine Monophosphate, Adenosine Diphosphate and Adenosine Triphosphate. In some embodiments, the nucleic acid molecule encodes a protein or silences a target gene.

In some embodiments, less than 5%, 10%, 15%, 20%, 25%, 30%, 35%, 40%, 45%, 50%, 55%, 60%, 65%, or 70% of loaded cargo (e.g., RNA) is on or associated with a nanoparticle exterior surface.

In some embodiments, the nucleic acid molecule is a circular RNA (i.e., circRNA), a type of single-stranded RNA which, unlike linear RNA, forms a covalently closed continuous loop. In circular RNA, the 3′ and 5′ ends normally present in an RNA molecule have been joined together. In some embodiments, the circular RNA code for proteins. In some embodiments, the circular RNA works as a gene regulator. Because circular RNAs do not have 5′ or 3′ ends, they are resistant to exonuclease-mediated degradation and are presumably more stable than most linear RNA in cells. Endogenous mammalian circular RNA has been linked to some diseases such as cancer (e.g. Su, M. et al Mol Cancer 18, 90 (2019)).

In some embodiments, the nucleic acid molecule is an interfering RNA (RNAi). The phenomenon of RNAi is discussed in greater detail, for example, in the following references: Elbashir et al., 2001, Genes Dev., 15:188; Fire et al., 1998, Nature, 391:806; Tabara et al., 1999, Cell, 99:123; Hammond et al., Nature, 2000, 404:293; Zamore et al., 2000, Cell, 101:25; Chakraborty, 2007, Curr. Drug Targets, 8:469; and Morris and Rossi, 2006, Gene Ther., 13:553. In certain embodiments, the nucleic acid molecule is a dsRNA (double-stranded RNA). In certain embodiments, the nucleic acid molecule is an siRNA (short interfering RNA). In certain embodiments, the nucleic acid molecule is an shRNA (short hairpin RNA). In certain embodiments, the nucleic acid molecule is an miRNA (micro RNA). Micro RNAs (miRNAs) are genomically encoded non-coding RNAs of about 21-23 nucleotides in length that help regulate gene expression, particularly during development. See, e.g., Bartel, 2004, Cell, 116:281; Novina and Sharp, 2004, Nature, 430:161; and U.S. Patent Publication 2005/0059005; also reviewed in Wang and Li, 2007, Front. Biosci., 12:3975; and Zhao, 2007, Trends Biochem. Sci., 32:189. In certain embodiments, the nucleic acid molecule is an antisense RNA.

In certain embodiments, the nucleic acid molecule may be provided as an antisense agent or RNA interference (RNAi). See, e.g., Fire et al., Nature 391:806-811, 1998. Antisense therapy is meant to include, e.g., administration or in situ provision of single- or double-stranded oligonucleotides or their derivatives which specifically hybridize, e.g., bind, under cellular conditions, with cellular mRNA and/or genomic DNA, or mutants thereof, so as to inhibit expression of the encoded protein, e.g., by inhibiting transcription and/or translation. See, e.g., Crooke “Molecular mechanisms of action of antisense drugs” Biochim. Biophys. Acta 1489(1):31-44, 1999; Crooke “Evaluating the mechanism of action of antiproliferative antisense drugs” Antisense Nucleic Acid Drug Dev. 10(2):123-126, discussion 127, 2000; Methods in Enzymology volumes 313-314, 1999. The binding may be by conventional base pair complementarity, or, for example, in the case of binding to DNA duplexes, through specific interactions in the major groove of the double helix (i.e., triple helix formation). See, e.g., Chan et al., J. Mol. Med. 75(4):267-282, 1997.

In some embodiments, dsRNA, dsDNA, ssDNA, siRNA, shRNA, miRNA, antisense RNA, and/or RNAi can be designed and/or predicted using one or more of a large number of available algorithms. To give but a few examples, the following resources can be utilized to design and/or predict polynucleotides: algorithms found at Alnylum Online, Dharmacon Online, OligoEngine Online, Molecula Online, Ambion Online, BioPredsi Online, RNAi Web Online, Chang Bioscience Online, Invitrogen Online, LentiWeb Online GenScript Online, Protocol Online; Reynolds et al., 2004, Nat. Biotechnol., 22:326; Naito et al., 2006, Nucleic Acids Res., 34:W448; Li et al., 2007, RNA, 13:1765; Yiu et al., 2005, Bioinformatics, 21:144; and Jia et al., 2006, BMC Bioinformatics, 7: 271.

The nucleic acid molecules may be of any size or sequence, and they may be single- or double-stranded. In certain embodiments, the nucleic acid molecule is greater than 10, 15, 20, 25, 30, 35, 40, 45, 50, 60, 70, 80, 90, or 100 base pairs long. In certain embodiments, the nucleic acid molecule is greater than 200, 300, 400, 500, 600, 700, 800, 900 or 1000 base pairs long and may be greater than 10,000 base pairs long. The nucleic acid molecule may be provided by any means known in the art. In certain embodiments, the nucleic acid molecule has been engineered using recombinant techniques. See, e.g., Ausubel et al., Current Protocols in Molecular Biology (John Wiley & Sons, Inc., New York, 1999); Molecular Cloning: A Laboratory Manual, 2nd Ed., ed. by Sambrook, Fritsch, and Maniatis (Cold Spring Harbor Laboratory Press: 1989). The nucleic acid molecule may also be obtained from natural sources and purified from contaminating components found normally in nature. The nucleic acid molecule may also be chemically synthesized in a laboratory. In certain embodiments, the nucleic acid molecule is synthesized using standard solid phase chemistry.

The nucleic acid molecule may be modified by chemical or biological means. In certain embodiments, these modifications lead to increased stability of the polynucleotide. Modifications include methylation, phosphorylation, end-capping, etc.

### 4. Pharmaceutical Compositions

In one aspect, the present disclosure provides a pharmaceutical composition comprising the nanoparticle complex disclosed herein. In some embodiments, the pharmaceutical composition comprises an effective amount of the nanoparticle complex disclosed herein and pharmaceutically acceptable carriers.

To facilitate delivery of the cargo, such as a nucleic acid (e.g., RNA), the nanoparticle complex can be formulated in pharmacological compositions where it is mixed with suitable excipients. Techniques for formulation and administration of drugs may be found in “Remington's Pharmaceutical Sciences,” Mack Publishing Co., Easton, Pa., latest edition.

Provided agents encapsulated in the lipid nanoparticles, such as a nucleic acid e.g., RNA and compositions containing the same, may be administered and dosed in accordance with current medical practice, taking into account the clinical condition of the subject, the site and method of administration, the scheduling of administration, the subject's age, sex, body weight and other factors relevant to clinicians of ordinary skill in the art. The “effective amount” for the purposes herein may be determined by such relevant considerations as are known to those of ordinary skill in experimental clinical research, pharmacological, clinical and medical arts. In some embodiments, the amount administered is effective to achieve at least some stabilization, improvement or elimination of symptoms and other indicators as are selected as appropriate measures of disease progress, regression or improvement by those of skill in the art. For example, a suitable amount and dosing regimen is one that causes at least transient protein (e.g., enzyme) production.

Also contemplated herein are lyophilized pharmaceutical compositions comprising one or more of the lipid nanoparticles disclosed herein and related methods for the use of such compositions as disclosed for example, in International Patent Application No. PCT/US2012/041663, filed Jun. 8, 2012, Publ. No. WO 2012/170889, the teachings of which are incorporated herein by reference in their entirety. For example, lyophilized pharmaceutical compositions according to the invention may be reconstituted prior to administration or can be reconstituted in vivo. For example, a lyophilized pharmaceutical composition can be formulated in an appropriate dosage form (e.g., an intradermal dosage form such as a disk, rod or membrane) and administered such that the dosage form is rehydrated over time in vivo by the individual's bodily fluids. In some embodiments, the constituents of the pharmaceutical composition, e.g., the lipid nanoparticle, the bifunctional binder, and the cargo are packaged separately, e.g., in different containers, and are mixed prior to the administration of the pharmaceutical composition.

Provided lipid nanoparticles and compositions may be administered to any desired tissue. In some embodiments, the agent, e.g., a nucleic acid delivered by provided lipid nanoparticles or compositions is expressed in the tissue in which the lipid nanoparticles and/or compositions were administered. In some embodiments, the nucleic acid delivered is expressed in a tissue different from the tissue in which the lipid nanoparticles and/or compositions were administered. Exemplary tissues in which delivered nucleic acid may be delivered and/or expressed include, but are not limited to the liver, kidney, heart, spleen, serum, brain, skeletal muscle, lymph nodes, skin, and/or cerebrospinal fluid.

### 5. Methods

### Methods of Making the Nanoparticle Complex

In one aspect, the present disclosure provides methods of making the nanoparticle complex provided herein.

The nanoparticle complexes of the present disclosure can be prepared by various techniques which are presently known in the art. For example, multilamellar vesicles (MLV) may be prepared according to conventional techniques, such as by depositing a selected lipid on the inside wall of a suitable container or vessel by dissolving the lipid in an appropriate solvent, and then evaporating the solvent to leave a thin film on the inside of the vessel or by spray drying. An aqueous phase may then added to the vessel with a vortexing motion which results in the formation of MLVs. Uni-lamellar vesicles (ULV) can then be formed by homogenization, sonication or extrusion of the multi-lamellar vesicles. In addition, unilamellar vesicles can be formed by detergent removal techniques.

In certain embodiments, in the nanoparticle complex disclosed herein, a cargo, such as a nucleic acid e.g., RNA, is associated on both the surface of the lipid nanoparticles and encapsulated within the same lipid nanoparticles. For example, during preparation of the nanoparticle complex disclosed herein, cationic lipid nanoparticles may associate with the RNA through electrostatic interactions. For example, during preparation of the nanoparticle complex disclosed herein, cationic lipid nanoparticle may associate with the RNA through electrostatic interactions.

In some embodiments, the nanoparticle complex disclosed herein comprise RNA encapsulated in a lipid nanoparticle. In some embodiments, the one or more RNA species may be encapsulated in the same lipid nanoparticle. In some embodiments, the one or more RNA species may be encapsulated in different lipid nanoparticles. In some embodiments, the RNA is encapsulated in one or more lipid nanoparticles, which differ in their lipid composition, molar ratio of lipid components, size, charge (Zeta potential), targeting ligands and/or combinations thereof. In some embodiments, the one or more lipid nanoparticles may have a different composition of cationic lipids, neutral lipid, PEG-modified lipid, biotin-modified lipoid and/or combinations thereof. In some embodiments the one or more lipid nanoparticles may have a different molar ratio of cationic lipid, neutral lipid, cholesterol and PEG-modified lipid and biotin-modified lipoid used to create the lipid nanoparticle. Microfluidic mixing is a common technique to encapsulate one or more nucleic acid molecules in LNPs. Microfluidic devices have been employed for liposome and LNP production, whose features, including continuous flow, precise control of reaction time, high-temperature controllability, and shorter diffusion distance in a microchannel, are advantageous for the production of microparticles (e.g., nanoparticle complexes as disclosed herein).

The incorporated nucleic acids may be completely or partially located in the interior space of the nanoparticle complex, within the bilayer membrane of the lipid nanoparticle, or associated with the exterior surface of the lipid nanoparticle membrane. The incorporation of a nucleic acid into lipid nanoparticles is also referred to herein as “encapsulation” or “loading”, wherein the nucleic acid is entirely contained within the interior space of the lipid nanoparticles. The purpose of incorporating a RNA into a transfer vehicle, such as a lipid nanoparticle, is often to protect the nucleic acid from an environment which may contain enzymes or chemicals that degrade nucleic acids and/or systems or receptors that cause the rapid excretion of the nucleic acids. Accordingly, in some embodiments, a suitable delivery vehicle is capable of enhancing the stability of the RNA contained therein and/or facilitate the delivery of RNA to the target cell or tissue.

Other exemplary methods are known in the art, such as those described in Lasic, et al., FEBS Lett., 312: 255-258, 1992, which is incorporated herein by reference.

### Methods of Delivering a Cargo to a Target Cell in a Subject

In one aspect, the present disclosure provides a method of delivering a cargo to a target cell in a subject. In some embodiments, the method comprises administering to the subject the nanoparticle complex of the present disclosure.

Suitable routes of administration include, for example, oral, sublingual, rectal, vaginal, transmucosal, pulmonary including intratracheal or inhaled, or intestinal administration; parenteral delivery, including intradermal, transdermal (topical), intramuscular, subcutaneous, intramedullary injections, as well as intrathecal, direct intraventricular, intravenous, intraperitoneal, and/or intranasal administration.

Alternately or additionally, agents encapsulated in the lipid nanoparticles, such as a nucleic acid e.g., RNA and compositions of the invention may be administered in a local rather than systemic manner, for example, via injection of the pharmaceutical composition directly into a targeted tissue, preferably in a sustained release formulation. Local delivery can be affected in various ways, depending on the tissue to be targeted. For example, aerosols containing compositions of the present invention can be inhaled (for nasal, tracheal, or bronchial delivery); compositions of the present invention can be injected into the site of injury, disease manifestation, or pain, for example; compositions can be provided in lozenges for oral, tracheal, or esophageal application; can be supplied in liquid, tablet or capsule form for administration to the stomach or intestines, can be supplied in suppository form for rectal or vaginal application; or can even be delivered to the eye by use of creams, drops, or even injection. Formulations containing provided compositions complexed with therapeutic molecules or ligands can even be surgically administered, for example in association with a polymer or other structure or substance that can allow the compositions to diffuse from the site of implantation to surrounding cells. Alternatively, they can be applied surgically without the use of polymers or supports.

In some embodiments, in the methods of the present disclosure, the nanoparticle complexes or the pharmaceutical compositions are formulated such that they are suitable for extended-release of the agent, e.g., RNA contained therein. Such extended-release compositions may be conveniently administered to a subject at extended dosing intervals. For example, in one embodiment, the method may comprise administering the nanoparticle complex or the pharmaceutical composition of the present disclosure to a subject twice day, daily or every other day. In a preferred embodiment, the nanoparticle complex or the pharmaceutical composition of the present disclosure is administered to a subject twice a week, once a week, every ten days, every two weeks, every three weeks, or more preferably every four weeks, once a month, every six weeks, every eight weeks, every other month, every three months, every four months, every six months, every eight months, every nine months or annually. Also contemplated are methods of administering nanoparticle complexes or the pharmaceutical compositions which are formulated for depot administration (e.g., intramuscularly, subcutaneously, intravitreally) to either deliver or release a mRNA over extended periods of time. Preferably, the extended-release means employed are combined with modifications made to the mRNA to enhance stability.

According to various embodiments, in the methods of delivering the cargo molecule to the target cell in the subject, the timing of expression of delivered cargo, e.g., RNA, can be tuned to suit a particular medical need. In some embodiments, the expression of the protein encoded by delivered RNA is detectable 1, 2, 3, 6, 12, 18, 24, 30, 36, 42, 48, 54, 60, 66, and/or 72 hours in serum or target tissues after a single administration of provided lipid nanoparticles or compositions. In some embodiments, the expression of the protein encoded by the RNA is detectable 1 day, 2 days, 3 days, 4 days, 5 days, 6 days, and/or 7 days in serum or target tissues after a single administration of provided lipid nanoparticles or compositions. In some embodiments, the expression of the protein encoded by the RNA is detectable 1 week, 2 weeks, 3 weeks, and/or 4 weeks in serum or target tissues after a single administration of provided lipid nanoparticles or compositions. In some embodiments, the expression of the protein encoded by the RNA is detectable after a month or longer after a single administration of provided lipid nanoparticles or compositions.

### 6. Pharmaceutical Use

In one aspect, the present disclosure provides use of the nanoparticle complex or the pharmaceutical composition in the manufacture of a medicament used in treating a disease/disorder/condition in a subject.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. A person skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

### Example 1

This example illustrates that LNP physical-chemical and basic biological properties were well maintained upon binder modification.

To prepare the ALC-biotin formulation, lipid mixture was prepared by dissolving lipid in ethanol in a molar ratio of ionizable lipid ALC-0315, cholesterol (cholesterol-biotin was used in ALC-biotin formulation), DSPC, DMG-PEG2000 and DSPE-PEG2000 (50:38.5:10:1.125:0.375). CircRNA coding firefly luciferase (Fluc) was dissolved in 10 mM citrate buffer (pH 4.0) at a concentration of 1.33 mg/mL. The two solutions were mixed with the ANP Dolomite system at an aqueous to ethanol ratio of 3:1 (v/v). The resulting formulation was dialyzed in three sessions against 1×PBS using dialysis cassette, and then filtered through a 0.22-μm filter.

LNP size and PDI measurement was completed by diluting the LNP sample 1:1000 in 0.1×PBS while surface charge measurement was completed on the LNP sample at 1:100 dilution in 0.1×PBS. Malvern Zetasizer was used for both the measurements. Ribogreen Analysis for encapsulation efficiency (EE) was completed following the protocol provided by the manufacturer. The LNP-Fluc was modified with commercial PEG binder RM105 (Sigma, anti-PEG Rbt IgG) or anti-biotin antibody (Cat #ab53494) by bulk-mixing the LNP and antibody in a 1:30 molar ratio respectively. Post-mixing, the vial was vortexed and incubated at room temp for 30 minutes, and then stored at 4° C. until dosing. For long term storage, the complexes were constituted with sucrose buffer and stored at −80° C. until dosing.

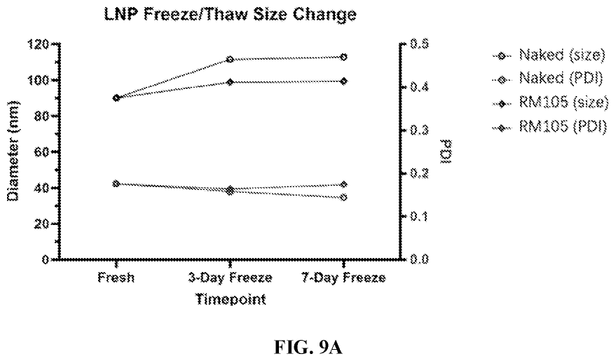

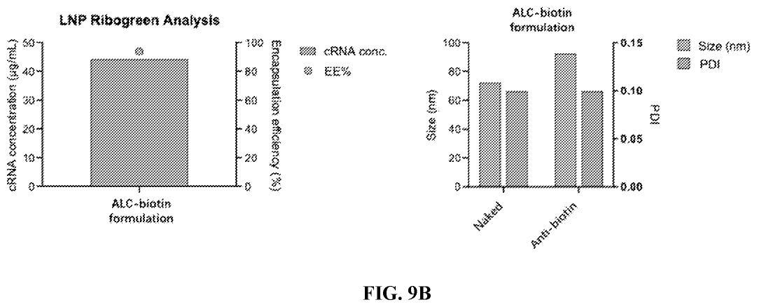

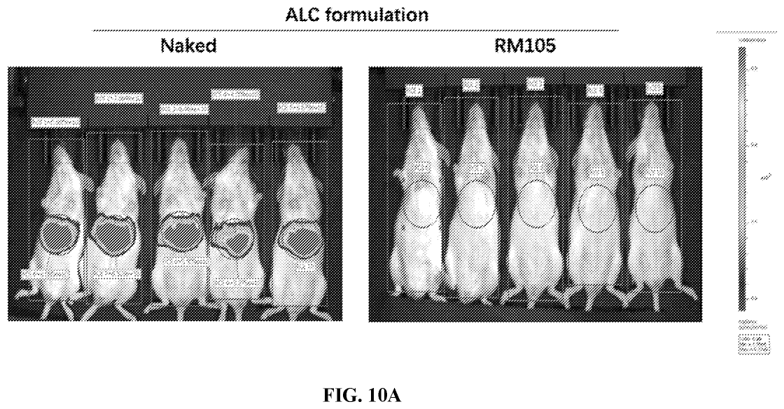

As shown in FIG. 9A and FIG. 9B, the particle size and PDI were well maintained upon binder modifications.

### Example 2

This example illustrates that binder modification leads to significant liver detargeting.

To investigate the effect of binder modification to LNP-RNA in vivo delivery, six-week-old female CD1 mice were ordered from Charles River. Mice were maintained on a 12 hr light cycle with free access to food and water for 96-168 hrs. Mice were dosed 10 mL/kg with 0.01 mg/mL Fluc circRNA formulated in either above mentioned ALC or ALC-biotin formulation with and without RM105 and anti-Biotin modifications. Six hours post dose, mice were dosed IP with 200 μL of 15 mg/mL D-Luciferin (Revity) in PBS. Eight minutes post dose, the mice are anesthetized with isoflurane and imaged on an IVIS Spectrum instrument. Regions of interest were defined and Total Flux and Average Radiance were calculated by the Living Image Software. Once whole-body images were taken, the mice were euthanized by cervical dislocation and tissues (liver, kidney and spleen) were removed and imaged independently.

As shown in FIG. 10A-10H, there is no significant changes in Flux level in the liver when dosed with naked LNP-Fluc with (sucrose-F/T) or without freeze and thaw (No sucrose; No F/T). In addition, compared to naked LNP-Fluc, both RM105 and anti-Biotin modification before ((1:30) RM105-Sucrose-FT) and after freeze and thaw (Sucrose-FT (1:30) RM105) significantly reduced Flux level in the liver, which demonstrated a potent liver detargeting effect regardless of freeze and thaw (FIGS. 101 and 10J).

### Example 3

This example illustrates the construction of bispecific binders for T cell and tumor cell targeted delivery.

To investigate targeted delivery driven by different formats of bispecific binders, multiple bispecific binders comprising two types of PEG binder (anti-polymer backbone and methoxy group of PEG), two types of targeted moiety (scFv and VHH), and multiple targets (CD5, CD7, CD8 and PD-L1) for T cell and tumor cell delivery were constructed. Name of binders and corresponding formats are shown in FIG. 11 and Table 1. Amino acid sequences of binder modules and bispecific binders are listed in Table 2 and 3. All of the binders were expressed by Expi293™ expression system (Thermo Fisher Scientific) following manufacturer's instructions and purified by affinity chromatography.

TABLE 1
Name and Corresponding Formats

| Name of Bispecific | PEG binder (Self- | | |
|---|---|---|---|
| binders | binder moiety) | Target binder | Format # |
| R3894 | NA | CD5 Clone 10 | 05 |
| M0047 | 16.4 | CD5 Clone 10 | 04 |
| M0108 | 14.3 | CD5 Clone 10 | 04 |
| M0065 | 16.4 | Isotype | 04 |
| M0066 | 16.4 | Isotype | 02 |
| M0110 | 14.3 | Isotype | 04 |
| R3893 | NA | CD7 | 05 |
| M0046 | 16.4 | CD7 | 04 |
| M0052 | 16.4 | CD7 | 02 |
| M0109 | 14.3 | CD7 | 04 |
| R4143 | NA | CD8 v144 | 06 |
| M0061 | 16.4 | CD8 v144 | 03 |
| M0076 | 14.3 | CD8 v144 | 03 |
| R1227 | NA | hPDL1 | 06 |
| R3603 | hClone6.3 | hPDL1 | 03 |
| R3602 | hClone6.3 | hPDL1 | 01 |

TABLE 2
Amino acid sequences of binder modules

| Name of | | |
|---|---|---|
| binder | | SEQ |
| modules | Amino acid sequences of binders | ID NO |
| VH of PEG | EVQLVESGGGLVKPGGSLKLSCAASGFTFSDYGMHWVRQAPEKG | 7 |
| Clone 16.4 | LEWVAYISSGSSTIYYADTVKGRFTISRDNAKNTLFLQMTNLRSED | |
| | TAMYYCVRAEITTVVADYWGQGTTLTVSS | |
| VL of PEG | QAVVTQESALTTSPGETVTLTCRLSTGAVTTSNYANWVQEKPDHL | 8 |
| Clone 16.4 | FTGLIGGTNNRAPGVPARFSGSLIGDKAALTITGAQTEDEARYFCV | |
| | LWYSNHWVFGGGTKLTVL | |
| VH of PEG | DVQLQESGPGLVKPSQSLSLTCSVTGYSITSGYYWNWVRQFPGNK | 15 |
| Clone 14.3 | LEWMGYISYDGSNNYKPSLKNRISITRDTSNNQFFLKLNSVTAEDT | |
| | ATYYCASGNGYDVGFAYWGQGTLVTVSA | |
| VL of PEG | QAVVTQESALTTSPGGTVILTCRSSTGAVTTSNYANWVQEKPDHL | 16 |
| Clone 14.3 | FTGLIGGTSNRTPGVPVRFSGSLIGDKAALTITGAQTEDDAMYFCA | |
| | LWYSTHYVFGGGTKVTVL | |
| VH of CD5 | EVQLLESGGGLVKPGGSLRLSCAASGFTFNNYTMNWVRQAPGKG | 17 |
| Clone10 | LEWVSSISSSSSYIYYADSVKGRFTISRDNAKNSLFLQMNSLRGED | |
| | TAVYYCARYFSGSAGDYWGQGTLVTVSS | |
| VL of CD5 | QSVVTQPPSVSGAPGQRVTISCTGSSSNIGAGYDVHWYQQLPGTA | 18 |
| Clone10 | PKLLIYGNINRPSGVPDRFSGSKSGTSASLAITGLQAEDEADYYCG | |
| | TWDNSLSAHYVFGTGTKVTVL | |
| VH of CD7 | EVQLVESGGGLVKPGGSLKLSCAASGLTFSSYAMSWVRQTPEKRL | 19 |
| | EWVASISSGGFTYYPDSVKGRFTISRDNARNILYLQMSSLRSEDTA | |
| | MYYCARDEVRGYLDVWGAGTTVTVSS | |
| VL of CD7 | DIQMTQTTSSLSASLGDRVTISCSASQGISNYLNWYQQKPDGTVKL | 20 |
| | LIYYTSSLHSGVPSRFSGSGSGTDYSLTISNLEPEDIATYYCQQYSK | |
| | LPYTFGGGTKLEIK | |
| VHH of | EVQLVESGGGLVQPGGSLRLSCAASGFTFDDYAIGWFRQAPGKGR | 21 |
| CD8 Clone | EGVACIRIFDRHTYYADSVKGRFTISSDNSKNTVYLQMNSLRAEDT | |
| v144 | ATYYCAAGSFWGCTRPEGDMDYFGQGTLVQVQSA | |
| VH of PEG | MGWSCIILFLVATATGVHSQVQLVQSGSELKKPGASVKVSCKASG | 22 |
| hClone6.3 | YTFKNYGMNWVRQAPGQGLEWMGWINTYTGQPIYANDFKGRFV | |
| | FSLDTSVSTAYLQISSLKAEDTAVYYCARDWGPYWGQGTLVTVSS | |
| VL of PEG | DIVMTQSPDSLAVSLGERATINCKSSQSVLYSSNQMNYLAWYQQK | 23 |
| hClone6.3 | PGQPPKLLIYWASTRESGVPDRFSGSGSGTDFTLTISSLQAEDVAV | |
| | YYCLQYLSSWTFGGGTKLEIK | |
| VHH of | QVQLVESGGGLVQPGGSLRLSCAASGRTFISYALGWFRQAPGQGL | 24 |
| hPDL1 | EAVAAISWSGSSTYYADSVKGRFTISRDNSKNTLYLQMNSLRAED | |
| | TAVYYCAADRTAATGSNLLRKYDYWGQGTLVTVSS | |

TABLE 3
Amino acid sequences of bi-specific binders

| Name of | |
|---|---|
| Bispecific | |
| binders | Amino acid sequences |
| R3894 | Light chain (SEQ ID NO: 25) |
| | ATMGWSCIILFLVATATGVHSQSVVTQPPSVSGAPGQRVTISCTGSSSNIGAG |
| | YDVHWYQQLPGTAPKLLIYGNINRPSGVPDRFSGSKSGTSASLAITGLQAED |
| | EADYYCGTWDNSLSAHYVFGTGTKVTVLRTVAAPSVFIFPPSDEQLKSGTA |
| | SVVCLLNNFYPREAKVQWKVDNALQSGNSQESVTEQDSKDSTYSLSSTLTL |
| | SKADYEKHKVYACEVTHQGLSSPVTKSFNRGEC |
| | Heavy chain (SEQ ID NO: 26) |
| | EVQLLESGGGLVKPGGSLRLSCAASGFTFNNYTMNWVRQAPGKGLEWVSS |
| | ISSSSSYIYYADSVKGRFTISRDNAKNSLFLQMNSLRGEDTAVYYCARYFSGS |
| | AGDYWGQGTLVTVSSASTKGPSVFPLAPSSKSTSGGTAALGCLVKDYFPEP |
| | VTVSWNSGALTSGVHTFPAVLQSSGLYSLSSVVTVPSSSLGTQTYICNVNHK |
| | PSNTKVDKRVEPKSCDKTHTCPPCPAPEAAGGPSVFLFPPKPKDTLMISRTPE |
| | VTCVVVDVSHEDPEVKFNWYVDGVEVHNAKTKPREEQYNSTYRVVSVLT |
| | VLHQDWLNGKEYKCKVSNKALPAPIEKTISKAKGQPREPQVYTLPPSREEM |
| | TKNQVSLTCLVKGFYPSDIAVEWESNGQPENNYKTTPPVLDSDGSFFLYSKL |
| | TVDKSRWQQGNVFSCSVMHEALHNHYTQKSLSLSPGK |
| M0047 | Light chain (SEQ ID NO: 27) |
| | QAVVTQESALTTSPGETVTLTCRLSTGAVTTSNYANWVQEKPDHLFTGLIGG |
| | TNNRAPGVPARFSGSLIGDKAALTITGAQTEDEARYFCVLWYSNHWVFGGG |
| | TKLTVLGQPKAAPSVTLFPPSSEELQANKATLVCLISDFYPGAVTVAWKADS |
| | SPVKAGVETTTPSKQSNNKYAASSYLSLTPEQWKSHRSYSCQVTHEGSTVE |
| | KTVAPTECS |
| | Heavy chain (SEQ ID NO: 28) |
| | EVQLVESGGGLVKPGGSLKLSCAASGFTFSDYGMHWVRQAPEKGLEWVAY |
| | ISSGSSTIYYADTVKGRFTISRDNAKNTLFLQMTNLRSEDTAMYYCVRAEIT |
| | TVVADYWGQGTTLTVSSASTKGPSVFPLAPSSKSTSGGTAALGCLVKDYFPE |
| | PVTVSWNSGALTSGVHTFPAVLQSSGLYSLSSVVTVPSSSLGTQTYICNVNH |
| | KPSNTKVDKRVEPKSCDKTHTCPPCPAPEAAGGPSVFLFPPKPKDTLMISRTP |
| | EVTCVVVDVSHEDPEVKFNWYVDGVEVHNAKTKPREEQYNSTYRVVSVL |
| | TVLHQDWLNGKEYKCKVSNKALPAPIEKTISKAKGQPREPQVYTLPPSREE |
| | MTKNQVSLTCLVKGFYPSDIAVEWESNGQPENNYKTTPPVLDSDGSFFLYSK |
| | LTVDKSRWQQGNVFSCSVMHEALHNHYTQKSLSLSPGAGGGGSGGGGSG |
| | GGGSQSVVTQPPSVSGAPGQRVTISCTGSSSNIGAGYDVHWYQQLPGTAPK |
| | LLIYGNINRPSGVPDRFSGSKSGTSASLAITGLQAEDEADYYCGTWDNSLSA |
| | HYVFGTGTKVTVLGGGGSGGGGSGGGGSEVQLLESGGGLVKPGGSLRLSC |
| | AASGFTFNNYTMNWVRQAPGKGLEWVSSISSSSSYIYYADSVKGRFTISRD |
| | NAKNSLFLQMNSLRGEDTAVYYCARYFSGSAGDYWGQGTLVTVSS |
| M0108 | Light chain (SEQ ID NO: 29) |
| | QAVVTQESALTTSPGGTVILTCRSSTGAVTTSNYANWVQEKPDHLFTGLIGG |
| | TSNRTPGVPVRFSGSLIGDKAALTITGAQTEDDAMYFCALWYSTHYVFGGG |
| | TKVTVLGQPKAAPSVTLFPPSSEELQANKATLVCLISDFYPGAVTVAWKADS |
| | SPVKAGVETTTPSKQSNNKYAASSYLSLTPEQWKSHRSYSCQVTHEGSTVE |
| | KTVAPTECS |
| | Heavy chain (SEQ ID NO: 30) |
| | DVQLQESGPGLVKPSQSLSLTCSVTGYSITSGYYWNWVRQFPGNKLEWMG |
| | YISYDGSNNYKPSLKNRISITRDTSNNQFFLKLNSVTAEDTATYYCASGNGY |
| | DVGFAYWGQGTLVTVSAASTKGPSVFPLAPSSKSTSGGTAALGCLVKDYFP |
| | EPVTVSWNSGALTSGVHTFPAVLQSSGLYSLSSVVTVPSSSLGTQTYICNVN |
| | HKPSNTKVDKRVEPKSCDKTHTCPPCPAPEAAGGPSVFLFPPKPKDTLMISR |
| | TPEVTCVVVDVSHEDPEVKFNWYVDGVEVHNAKTKPREEQYNSTYRVVS |
| | VLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGAGGGGSGGGGS |
| | GGGGSQSVVTQPPSVSGAPGQRVTISCTGSSSNIGAGYDVHWYQQLPGTAP |
| | KLLIYGNINRPSGVPDRFSGSKSGTSASLAITGLQAEDEADYYCGTWDNSLS |
| | AHYVFGTGTKVTVLGGGGSGGGGSGGGGSEVQLLESGGGLVKPGGSLRLS |
| | CAASGFTFNNYTMNWVRQAPGKGLEWVSSISSSSSYIYYADSVKGRFTISR |
| | DNAKNSLFLQMNSLRGEDTAVYYCARYFSGSAGDYWGQGTLVTVSS |
| M0065 | Light chain (SEQ ID NO: 31) |
| | QAVVTQESALTTSPGETVTLTCRLSTGAVTTSNYANWVQEKPDHLFTGLIGG |
| | TNNRAPGVPARFSGSLIGDKAALTITGAQTEDEARYFCVLWYSNHWVFGGG |
| | TKLTVLGQPKAAPSVTLFPPSSEELQANKATLVCLISDFYPGAVTVAWKADS |
| | SPVKAGVETTTPSKQSNNKYAASSYLSLTPEQWKSHRSYSCQVTHEGSTVE |
| | KTVAPTECS |
| | Heavy chain (SEQ ID NO: 32) |
| | EVQLVESGGGLVKPGGSLKLSCAASGFTFSDYGMHWVRQAPEKGLEWVAY |
| | ISSGSSTIYYADTVKGRFTISRDNAKNTLFLQMTNLRSEDTAMYYCVRAEIT |
| | TVVADYWGQGTTLTVSSASTKGPSVFPLAPSSKSTSGGTAALGCLVKDYFPE |
| | PVTVSWNSGALTSGVHTFPAVLQSSGLYSLSSVVTVPSSSLGTQTYICNVNH |
| | KPSNTKVDKRVEPKSCDKTHTCPPCPAPEAAGGPSVFLFPPKPKDTLMISRTP |
| | EVTCVVVDVSHEDPEVKFNWYVDGVEVHNAKTKPREEQYNSTYRVVSVL |
| | TVLHQDWLNGKEYKCKVSNKALPAPIEKTISKAKGQPREPQVYTLPPSREE |
| | MTKNQVSLTCLVKGFYPSDIAVEWESNGQPENNYKTTPPVLDSDGSFFLYSK |
| | LTVDKSRWQQGNVFSCSVMHEALHNHYTQKSLSLSPGGGGGSGGGGSGG |
| | GGSDVVMTQTPLSLPVSLGDQASISCRSSQSLVHSNGNTYLRWYLQKPGQS |
| | PKVLIYKVSNRFSGVPDRFSGSGSGTDFTLKISRVEAEDLGVYFCSQSTHVP |
| | WTFGGGTKLEIKGGGGSGGGGSGGGGSGGGGSEVKLDETGGGLVQPGRP |
| | MKLSCVASGFTFSDYWMNWVRQSPEKGLEWVAQIRNKPYNYETYYSDSV |
| | KGRFTISRDDSKSSVYLQMNNLRVEDMGIYYCTGSYYGMDYWGQGTSVT |
| | VS |
| M0066 | Single chain (SEQ ID NO: 33) |
| | EVQLVESGGGLVKPGGSLKLSCAASGFTFSDYGMHWVRQAPEKGLEWVAY |
| | ISSGSSTIYYADTVKGRFTISRDNAKNTLFLQMTNLRSEDTAMYYCVRAEIT |
| | TVVADYWGQGTTLTVSSGGGGSGGGGSGGGGSQAVVTQESALTTSPGETV |
| | TLTCRLSTGAVTTSNYANWVQEKPDHLFTGLIGGTNNRAPGVPARFSGSLIG |
| | DKAALTITGAQTEDEARYFCVLWYSNHWVFGGGTKLTVLSGGGGSDVVMT |
| | QTPLSLPVSLGDQASISCRSSQSLVHSNGNTYLRWYLQKPGQSPKVLIYKVS |
| | NRFSGVPDRFSGSGSGTDFTLKISRVEAEDLGVYFCSQSTHVPWTFGGGTKL |
| | EIKGGGGSGGGGSGGGGSGGGGSEVKLDETGGGLVQPGRPMKLSCVASGF |
| | TFSDYWMNWVRQSPEKGLEWVAQIRNKPYNYETYYSDSVKGRFTISRDDS |
| | KSSVYLQMNNLRVEDMGIYYCTGSYYGMDYWGQGTSVTVSWGQGTSVT |
| | VS |
| M0110 | Light chain (SEQ ID NO: 34) |
| | QAVVTQESALTTSPGGTVILTCRSSTGAVTTSNYANWVQEKPDHLFTGLIGG |
| | TSNRTPGVPVRFSGSLIGDKAALTITGAQTEDDAMYFCALWYSTHYVFGGG |
| | TKVTVLGQPKAAPSVTLFPPSSEELQANKATLVCLISDFYPGAVTVAWKADS |
| | SPVKAGVETTTPSKQSNNKYAASSYLSLTPEQWKSHRSYSCQVTHEGSTVE |
| | KTVAPTECS |
| | Heavy chain (SEQ ID NO: 35) |
| | DVQLQESGPGLVKPSQSLSLTCSVTGYSITSGYYWNWVRQFPGNKLEWMG |
| | YISYDGSNNYKPSLKNRISITRDTSNNQFFLKLNSVTAEDTATYYCASGNGY |
| | DVGFAYWGQGTLVTVSAASTKGPSVFPLAPSSKSTSGGTAALGCLVKDYFP |
| | EPVTVSWNSGALTSGVHTFPAVLQSSGLYSLSSVVTVPSSSLGTQTYICNVN |
| | HKPSNTKVDKRVEPKSCDKTHTCPPCPAPEAAGGPSVFLFPPKPKDTLMISR |
| | TPEVTCVVVDVSHEDPEVKFNWYVDGVEVHNAKTKPREEQYNSTYRVVS |
| | VLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGAGGGGSGGGGS |
| | GGGGSDVVMTQTPLSLPVSLGDQASISCRSSQSLVHSNGNTYLRWYLQKPG |
| | QSPKVLIYKVSNRFSGVPDRFSGSGSGTDFTLKISRVEAEDLGVYFCSQSTH |
| | VPWTFGGGTKLEIKGGGGGGGGSGGGGSGGGGSEVKLDETGGGLVQPGR |
| | PMKLSCVASGFTFSDYWMNWVRQSPEKGLEWVAQIRNKPYNYETYYSDSV |
| | KGRFTISRDDSKSSVYLQMNNLRVEDMGIYYCTGSYYGMDYWGQGTSVT |
| | VS |
| R3893 | Light chain (SEQ ID NO: 36) |
| | DIQMTQTTSSLSASLGDRVTISCSASQGISNYLNWYQQKPDGTVKLLIYYTS |
| | SLHSGVPSRFSGSGSGTDYSLTISNLEPEDIATYYCQQYSKLPYTFGGGTKLEI |
| | KRTVAAPSVFIFPPSDEQLKSGTASVVCLLNNFYPREAKVQWKVDNALQSG |
| | NSQESVTEQDSKDSTYSLSSTLTLSKADYEKHKVYACEVTHQGLSSPVTKSF |
| | NRGEC |
| | Heavy chain (SEQ ID NO: 37) |
| | EVQLVESGGGLVKPGGSLKLSCAASGLTFSSYAMSWVRQTPEKRLEWVASI |
| | SSGGFTYYPDSVKGRFTISRDNARNILYLQMSSLRSEDTAMYYCARDEVRG |
| | YLDVWGAGTTVTVSSASTKGPSVFPLAPSSKSTSGGTAALGCLVKDYFPEP |
| | VTVSWNSGALTSGVHTFPAVLQSSGLYSLSSVVTVPSSSLGTQTYICNVNHK |
| | PSNTKVDKRVEPKSCDKTHTCPPCPAPEAAGGPSVFLFPPKPKDTLMISRTPE |
| | VTCVVVDVSHEDPEVKFNWYVDGVEVHNAKTKPREEQYNSTYRVVSVLT |
| | VLHQDWLNGKEYKCKVSNKALPAPIEKTISKAKGQPREPQVYTLPPSREEM |
| | TKNQVSLTCLVKGFYPSDIAVEWESNGQPENNYKTTPPVLDSDGSFFLYSKL |
| | TVDKSRWQQGNVFSCSVMHEALHNHYTQKSLSLSPGK |
| M0046 | Light chain (SEQ ID NO: 38) |
| | QAVVTQESALTTSPGETVTLTCRLSTGAVTTSNYANWVQEKPDHLFTGLIGG |
| | TNNRAPGVPARFSGSLIGDKAALTITGAQTEDEARYFCVLWYSNHWVFGGG |
| | TKLTVLGQPKAAPSVTLFPPSSEELQANKATLVCLISDFYPGAVTVAWKADS |
| | SPVKAGVETTTPSKQSNNKYAASSYLSLTPEQWKSHRSYSCQVTHEGSTVE |
| | KTVAPTECS |
| | Heavy chain (SEQ ID NO: 39) |
| | EVQLVESGGGLVKPGGSLKLSCAASGFTFSDYGMHWVRQAPEKGLEWVAY |
| | ISSGSSTIYYADTVKGRFTISRDNAKNTLFLQMTNLRSEDTAMYYCVRAEIT |
| | TVVADYWGQGTTLTVSSASTKGPSVFPLAPSSKSTSGGTAALGCLVKDYFPE |
| | PVTVSWNSGALTSGVHTFPAVLQSSGLYSLSSVVTVPSSSLGTQTYICNVNH |
| | KPSNTKVDKRVEPKSCDKTHTCPPCPAPEAAGGPSVFLFPPKPKDTLMISRTP |
| | EVTCVVVDVSHEDPEVKFNWYVDGVEVHNAKTKPREEQYNSTYRVVSVL |
| | TVLHQDWLNGKEYKCKVSNKALPAPIEKTISKAKGQPREPQVYTLPPSREE |
| | MTKNQVSLTCLVKGFYPSDIAVEWESNGQPENNYKTTPPVLDSDGSFFLYSK |
| | LTVDKSRWQQGNVFSCSVMHEALHNHYTQKSLSLSPGGGGGSGGGGSGG |
| | GGSDIQMTQTTSSLSASLGDRVTISCSASQGISNYLNWYQQKPDGTVKLLIY |
| | YTSSLHSGVPSRFSGSGSGTDYSLTISNLEPEDIATYYCQQYSKLPYTFGGGT |
| | KLEIKGGGGSGGGGSGGGGSGGGGSEVQLVESGGGLVKPGGSLKLSCAAS |
| | GLTFSSYAMSWVRQTPEKRLEWVASISSGGFTYYPDSVKGRFTISRDNARNI |
| | LYLQMSSLRSEDTAMYYCARDEVRGYLDVWGAGTTVTVSS |
| M0052 | Single chain (SEQ ID NO: 40) |
| | EVQLVESGGGLVKPGGSLKLSCAASGFTFSDYGMHWVRQAPEKGLEWVAY |
| | ISSGSSTIYYADTVKGRFTISRDNAKNTLFLQMTNLRSEDTAMYYCVRAEIT |
| | TVVADYWGQGTTLTVSSGGGGSGGGGSGGGGSQAVVTQESALTTSPGETV |
| | TLTCRLSTGAVTTSNYANWVQEKPDHLFTGLIGGTNNRAPGVPARFSGSLIG |
| | DKAALTITGAQTEDEARYFCVLWYSNHWVFGGGTKLTVLSGGGGSEVQLV |
| | ESGGGLVKPGGSLKLSCAASGLTFSSYAMSWVRQTPEKRLEWVASISSGGFT |
| | YYPDSVKGRFTISRDNARNILYLQMSSLRSEDTAMYYCARDEVRGYLDVW |
| | GAGTTVTVSSGGGGSGGGGSGGGGSDIQMTQTTSSLSASLGDRVTISCSAS |
| | QGISNYLNWYQQKPDGTVKLLIYYTSSLHSGVPSRFSGSGSGTDYSLTISNL |
| | EPEDIATYYCQQYSKLPYTFGGGTKLEIK |
| M0109 | Light chain (SEQ ID NO: 41) |
| | QAVVTQESALTTSPGGTVILTCRSSTGAVTTSNYANWVQEKPDHLFTGLIGG |
| | TSNRTPGVPVRFSGSLIGDKAALTITGAQTEDDAMYFCALWYSTHYVFGGG |
| | TKVTVLGQPKAAPSVTLFPPSSEELQANKATLVCLISDFYPGAVTVAWKADS |
| | SPVKAGVETTTPSKQSNNKYAASSYLSLTPEQWKSHRSYSCQVTHEGSTVE |
| | KTVAPTECS |
| | Heavy chain (SEQ ID NO: 42) |
| | DVQLQESGPGLVKPSQSLSLTCSVTGYSITSGYYWNWVRQFPGNKLEWMG |
| | YISYDGSNNYKPSLKNRISITRDTSNNQFFLKLNSVTAEDTATYYCASGNGY |
| | DVGFAYWGQGTLVTVSAASTKGPSVFPLAPSSKSTSGGTAALGCLVKDYFP |
| | EPVTVSWNSGALTSGVHTFPAVLQSSGLYSLSSVVTVPSSSLGTQTYICNVN |
| | HKPSNTKVDKRVEPKSCDKTHTCPPCPAPEAAGGPSVFLFPPKPKDTLMISR |
| | TPEVTCVVVDVSHEDPEVKFNWYVDGVEVHNAKTKPREEQYNSTYRVVS |
| | VLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGAGGGGSGGGGS |
| | GGGGSDIQMTQTTSSLSASLGDRVTISCSASQGISNYLNWYQQKPDGTVKL |
| | LIYYTSSLHSGVPSRFSGSGSGTDYSLTISNLEPEDIATYYCQQYSKLPYTFGG |
| | GTKLEIKGGGGSGGGGSGGGGSGGGGSEVQLVESGGGLVKPGGSLKLSCA |
| | ASGLTFSSYAMSWVRQTPEKRLEWVASISSGGFTYYPDSVKGRFTISRDNAR |
| | NILYLQMSSLRSEDTAMYYCARDEVRGYLDVWGAGTTVTVSS |
| R4143 | Single chain (SEQ ID NO: 43) |
| | EVqLVESGGGLVQPGGSLRLSCAASGFTFDDYAIGWFRQAPGKGREGVACI |
| | RIFDRHTYYADSVKGRFTISSDNSKNTVYLQMNSLRAEDTATYYCAAGSFW |
| | GCTRPEGDMDYFGQGTLVQVQSAEPKSSDKTHTCPPCPAPEAAGGPSVFLF |
| | PPKPKDTLMISRTPEVTCVVVDVSHEDPEVKFNWYVDGVEVHNAKTKPRE |
| | EQYNSTYRVVSVLTVLHQDWLNGKEYKCKVSNKALPAPIEKTISKAKGQPR |
| | EPQVYTLPPSREEMTKNQVSLTCLVKGFYPSDIAVEWESNGQPENNYKTTPP |
| | VLDSDGSFFLYSKLTVDKSRWQQGNVFSCSVMHEALHNHYTQKSLSLSPGA |
| M0061 | Light chain (SEQ ID NO: 44) |
| | QAVVTQESALTTSPGETVTLTCRLSTGAVTTSNYANWVQEKPDHLFTGLIGG |
| | TNNRAPGVPARFSGSLIGDKAALTITGAQTEDEARYFCVLWYSNHWVFGGG |
| | TKLTVLGQPKAAPSVTLFPPSSEELQANKATLVCLISDFYPGAVTVAWKADS |
| | SPVKAGVETTTPSKQSNNKYAASSYLSLTPEQWKSHRSYSCQVTHEGSTVE |
| | KTVAPTECS |
| | Heavy chain (SEQ ID NO: 45) |
| | EVQLVESGGGLVKPGGSLKLSCAASGFTFSDYGMHWVRQAPEKGLEWVAY |
| | ISSGSSTIYYADTVKGRFTISRDNAKNTLFLQMTNLRSEDTAMYYCVRAEIT |
| | TVVADYWGQGTTLTVSSASTKGPSVFPLAPSSKSTSGGTAALGCLVKDYFPE |
| | PVTVSWNSGALTSGVHTFPAVLQSSGLYSLSSVVTVPSSSLGTQTYICNVNH |
| | KPSNTKVDKRVEPKSCDKTHTCPPCPAPEAAGGPSVFLFPPKPKDTLMISRTP |
| | EVTCVVVDVSHEDPEVKFNWYVDGVEVHNAKTKPREEQYNSTYRVVSVL |
| | TVLHQDWLNGKEYKCKVSNKALPAPIEKTISKAKGQPREPQVYTLPPSREE |
| | MTKNQVSLTCLVKGFYPSDIAVEWESNGQPENNYKTTPPVLDSDGSFFLYSK |
| | LTVDKSRWQQGNVFSCSVMHEALHNHYTQKSLSLSPGKGGGGSGGGGSG |
| | GGGSEVQLVESGGGLVQPGGSLRLSCAASGFTFDDYAIGWFRQAPGKGREG |
| | VACIRIFDRHTYYADSVKGRFTISSDNSKNTVYLQMNSLRAEDTATYYCAAG |
| | SFWGCTRPEGDMDYFGQGTLVQVQSA |
| M0076 | Light chain (SEQ ID NO: 46) |
| | QAVVTQESALTTSPGGTVILTCRSSTGAVTTSNYANWVQEKPDHLFTGLIGG |
| | TSNRTPGVPVRFSGSLIGDKAALTITGAQTEDDAMYFCALWYSTHYVFGGG |
| | TKVTVLGQPKAAPSVTLFPPSSEELQANKATLVCLISDFYPGAVTVAWKADS |
| | SPVKAGVETTTPSKQSNNKYAASSYLSLTPEQWKSHRSYSCQVTHEGSTVE |
| | KTVAPTECS |
| | Heavy chain (SEQ ID NO: 47) |
| | DVQLQESGPGLVKPSQSLSLTCSVTGYSITSGYYWNWVRQFPGNKLEWMG |
| | YISYDGSNNYKPSLKNRISITRDTSNNQFFLKLNSVTAEDTATYYCASGNGY |
| | DVGFAYWGQGTLVTVSAASTKGPSVFPLAPSSKSTSGGTAALGCLVKDYFP |
| | EPVTVSWNSGALTSGVHTFPAVLQSSGLYSLSSVVTVPSSSLGTQTYICNVN |
| | HKPSNTKVDKRVEPKSCDKTHTCPPCPAPEAAGGPSVFLFPPKPKDTLMISR |
| | TPEVTCVVVDVSHEDPEVKFNWYVDGVEVHNAKTKPREEQYNSTYRVVS |
| | VLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGGS |
| | GGGGSEVQLVESGGGLVQPGGSLRLSCAASGFTFDDYAIGWFRQAPGKGRE |
| | GVACIRIFDRHTYYADSVKGRFTISSDNSKNTVYLQMNSLRAEDTATYYCAA |
| | GSFWGCTRPEGDMDYFGQGTLVQVQSA |
| R1227 | Heavy chain (SEQ ID NO: 48) |
| | QVQLVESGGGLVQPGGSLRLSCAASGRTFISYALGWFRQAPGQGLEAVAAIS |
| | WSGSSTYYADSVKGRFTISRDNSKNTLYLQMNSLRAEDTAVYYCAADRTAA |
| | TGSNLLRKYDYWGQGTLVTVSSVPRDCGCKPCICTVPEVSSVFIFPPKPKDV |
| | LTITLTPKVTCVVVDISKDDPEVQFSWFVDDVEVHTAQTQPREEQFNSTFRS |
| | VSELPIMHQDWLNGKEFKCRVNSAAFPAPIEKTISKTKGRPKAPQVYTIPPPK |
| | EQMAKDKVSLTCMITNFFPEDITVEWQWNGQPAENYKNTQPIMDTDGSYF |
| | VYSKLNVQKSNWEAGNTFTCSVLHEGLHNHHTEKSLSHSPGK |
| R3603 | Light chain (SEQ ID NO: 49) |
| | DIVMTQSPDSLAVSLGERATINCKSSQSVLYSSNQMNYLAWYQQKPGQPPK |
| | LLIYWASTRESGVPDRFSGSGSGTDFTLTISSLQAEDVAVYYCLQYLSSWTFG |
| | GGTKLEIKRTVAAPSVFIFPPSDEQLKSGTASVVCLLNNFYPREAKVQWKVD |
| | NALQSGNSQESVTEQDSKDSTYSLSSTLTLSKADYEKHKVYACEVTHQGLS |
| | SPVTKSFNRGEC |
| | Heavy chain (SEQ ID NO: 50) |
| | QVQLVQSGSELKKPGASVKVSCKASGYTFKNYGMNWVRQAPGQGLEWM |
| | GWINTYTGQPIYANDFKGRFVFSLDTSVSTAYLQISSLKAEDTAVYYCARDW |
| | GPYWGQGTLVTVSSASTKGPSVFPLAPSSKSTSGGTAALGCLVKDYFPEPVT |
| | VSWNSGALTSGVHTFPAVLQSSGLYSLSSVVTVPSSSLGTQTYICNVNHKPS |
| | NTKVDKRVEPKSCDKTHTCPPCPAPEAAGGPSVFLFPPKPKDTLMISRTPEV |
| | TCVVVDVSHEDPEVKFNWYVDGVEVHNAKTKPREEQYNSTYRVVSVLTV |
| | LHQDWLNGKEYKCKVSNKALPAPIEKTISKAKGQPREPQVYTLPPSREEMT |
| | KNQVSLTCLVKGFYPSDIAVEWESNGQPENNYKTTPPVLDSDGSFFLYSKLT |
| | VDKSRWQQGNVFSCSVMHEALHNHYTQKSLSLSPGKGGGGSGGGGSGGG |
| | GSQVQLVESGGGLVQPGGSLRLSCAASGRTFISYALGWFRQAPGQGLEAVA |
| | AISWSGSSTYYADSVKGRFTISRDNSKNTLYLQMNSLRAEDTAVYYCAADR |
| | TAATGSNLLRKYDYWGQGTLVTVSS |
| R3602 | Single chain (SEQ ID NO: 51) |
| | MGWSCIILFLVATATGVHSQVQLVQSGSELKKPGASVKVSCKASGYTFKNY |
| | GMNWVRQAPGQGLEWMGWINTYTGQPIYANDFKGRFVFSLDTSVSTAYLQ |
| | ISSLKAEDTAVYYCARDWGPYWGQGTLVTVSSGGGGSGGGGSGGGGSDIV |
| | MTQSPDSLAVSLGERATINCKSSQSVLYSSNQMNYLAWYQQKPGQPPKLLI |
| | YWASTRESGVPDRFSGSGSGTDFTLTISSLQAEDVAVYYCLQYLSSWTFGGG |
| | TKLEIKGGGGSGGGGSGGGGSQVQLVESGGGLVQPGGSLRLSCAASGRTFIS |
| | YALGWFRQAPGQGLEAVAAISWSGSSTYYADSVKGRFTISRDNSKNTLYLQ |
| | MNSLRAEDTAVYYCAADRTAATGSNLLRKYDYWGQGTLVTVSS |

### Example 4

This example illustrates the preparation and characterization of LNP-RNA with and without modification by above mentioned binders.

To prepare LNP-RNA, mRNA was synthesized by one-pot mRNA Co-transcription Kit (XbaI) (Syngenebio) following manufacturer's instructions. Circular RNA was synthesized in-house using the method published in Wesselhoeft R A, et al. (RNA Circularization Diminishes Immunogenicity and Can Extend Translation Duration In Vivo. Mol Cell. 2019 May 2; 74(3):508-520.e4). siRNA was purchased from Genscript. The RNAs were encapsulated by the following formulations.

TABLE 4
Recipes of LNP-RNA

| Formulation name | Recipe |
|---|---|
| ALC-DOPE | lipid mixture was prepared by dissolving lipid in ethanol in a molar ratio of |
| | ionizable lipid ALC-0315, cholesterol, DOPE, DMG-PEG2000 |
| | (50:38.5:10:1.5). mRNA or circRNA was dissolved in 10 mM citrate buffer |
| | (pH 4.0) as 100 μg/mL and the required total amount was prepare based on |
| | lipid to RNA ratio (w/w) of 24.4:1. |
| ALC | lipid mixture was prepared by dissolving lipid in ethanol in a molar ratio of |
| | ionizable lipid ALC-0315, cholesterol, DSPC, DMG-PEG2000 and DSPE- |
| | PEG2000 (50:38.5:10:1.125:0.375). mRNA or circRNA was dissolved in 10 |
| | mM citrate buffer (pH 4.0) as 100 μg/mL and the required total amount was |
| | prepare based on lipid to RNA ratio (w/w) of 24.4:1. |
| M3413 | lipid mixture was prepared by dissolving lipid in ethanol in a molar ratio of |
| | SM102, cholesterol, DOPE, DMG-PEG2000, DSPE-PEG2000 and 18:1PG |
| | (45:34.65:9:1.01:0.34:10). mRNA or circRNA was dissolved in 10 mM citrate |
| | buffer (pH 4.0) as 100 μg/mL and the required total amount was prepare based |
| | on lipid to RNA ratio (w/w) of 21.9. 18:1PG is 1,2-dioleoyl-sn-glycero-3- |
| | phospho-(1′-rac-glycerol) (sodium salt). |
| SM3 | lipid mixture was prepared by dissolving lipid in ethanol in a molar ratio of |
| | Lipid5, cholesterol, DSPC, DMG-PEG2000, DSPE-PEG2000 and 18:1PG |
| | (45:34.65:9:1.01:0.34:10). mRNA or circRNA was dissolved in 10 mM citrate |
| | buffer (pH 4.0) as 100 μg/mL and the required total amount was prepare based |
| | on lipid to RNA ratio (w/w) of 21.9. |
| SM102 | lipid mixture was prepared by dissolving lipid in ethanol in a molar ratio of |
| | SM102, cholesterol, DSPC and DMG-PEG2000 (50:38.5:10:1.5). siRNA was |
| | dissolved in 10 mM citrate buffer (pH 4.0) as 100 μg/mL and the required total |
| | amount was prepare based on an N/P ratio of 6. |

For encapsulation, lipid mixture and RNA solution were mixed with microfluidics at an aqueous to ethanol ratio of 3:1 (v/v). The resulting formulation was dialyzed against 1×PBS using Slide-A-Lyzer dialysis cassette for 18h, filtered through a 0.22-μm filter and then concentrated using Amicon ultra-centrifugal filters.

Particle size, PDI and zeta potential were measured by Malvern Zetasizer Pro. Encapsulation efficiency (EE) were measured by Quant-it™ RiboGreen RNA Assay Kit following manufacture's protocol.

To prepare binder-LNP-RNA complexes, binders in 01-02 format and LNP-RNA were mixed in a protein to RNA mass ratio of 1.5:1 with binder concentration fixed to 150 μg/mL, while binders in 03-06 formats and LNP-RNA were mixed in a protein to RNA mass ratio of 1:1.5 with binder concentration fixed to 70 μg/mL. The mixture was then incubated at room temperature for 2 hours followed by storage at 4° C. overnight. Particle size and PDI were measured by DLS in 24 hours and binder-LNP-RNA complexes were used in 48 hours. As shown in FIG. 12A-12E, particle size and PDI were well maintained with binder modifications.

### Example 5

This example illustrates that in vitro targeted delivery efficiency of LNP was enhanced by multiple bispecific binders.

To measure binder driven targeted delivery in vitro, MC38, MC38-hPDL1 (MC38 with human PD-L1 overexpression, generated by lentiviral transduction), Jurkat and hPBMC-T cells were used. hPBMC-T were isolated by EasySep™ Human T Cell Isolation Kit (STEMCELL Technologies) activated by CD3/CD28 beads (Dynabeads™) following manufacturer's protocol. MC38 was cultured in DMEM (Gibco) supplemented with 10% FBS (Gibco). Jurkat T cells were cultured using 1640RPMI (Gibco) supplemented with 10% FBS (Gibco). hPBMC-T was cultured in X-Vivo 15 (Lonza) supplemented with 300 IU IL-2 (Sihuanshengwu) and 5% Human AB serum (GEMINI). MC38, MC38-hPDL1 were plated as 10000 cells/well while Jurkat and hPBMC-T as 100000/well in 96-well-plate for incubation of naked LNP-circGFP, binder-LNP-circGFP or binder-LNP-circFluc complexes as 0.5 to 100 ng RNA per well for 24 hours. To demonstrate the enhanced targeted delivery is driven by specific binding to targets on T cells, anti-CD5/7/8 mAb were added respectively as 10 μg/mL one hour before incubation of binder-LNP-circGFP complexes for blocking interaction between bispecific binder and T cells. Percentage of GFP positive cells were measured by flow-cytometry under FITC channel. Expression levels of Fluc were measured by luminescence assay using firefly luciferin (Beyotime technology).

Regardless of LNP formulations, epitopes of PEG binders, types of target binders, targets and bispecific binder formats, delivery efficiency to Jurkat and hPBMC-T cells were dramatically enhanced by bispecific binder modifications, and the enhancement was abolished by blocking T cell surface targets including CD5, CD7 and CD8 (FIG. 13A-15C). Similar phenotypes were observed in human PD-L1 tumor models (FIGS. 16A and 16B). These results demonstrated that targeted delivery efficiency of LNP was potently increased by versatile bispecific binder modifications.

### Example 6

This example illustrates that in vivo targeted T cell delivery was enhanced by multiple bispecific binders.

To validate liver detargeting mediated by bispecific binders, wild-type mice were dosed with naked LNP-circFluc and CD5xPEG-LNP-circFluc as 0.2 mg/kg and imaged at 6 hours post dosage. Consistent with results presented in FIG. 13A-13C, CD5xPEG modification significantly reduced liver delivery (FIGS. 17B and 17C), which indicated potent liver detargeting.

To investigate binder driven targeted delivery in vivo, genetic engineered mice expressing human CD5 (hCD5-mice) were used for CD5XPEG bispecific binder driven targeted delivery experiments, while hPBMC humanized NOG mice (hPBMC-NOG) were applied for CD7XPEG bispecific binder driven targeted delivery experiments.

For hCD5-mice model, 0.2 mg/kg of naked LNP-circGFP, isotype-LNP-circGFP and CD5xPEG-LNP-circGFP were dosed by intravenous injection (i.v.). For hPBMC-NOG model, 5×10⁶ hPBMC were engrafted 3 weeks before dosage and 0.2 mg/kg of naked LNP-circGFP, isotype-LNP-circGFP and CD7xPEG-LNP-circGFP were dosed by i.v.

For both models, spleen and PBMC were collected for tissue isolation and flow-cytometry analysis of cell identity and GFP expression 48 hours post dosage. As shown in FIG. 18A-19D, delivery of LNP-circGFP into mouse spleen and PBMC T cells were both potently increased by either CD5xPEG or CD7xPEG binder modifications.

### Example 7

This example illustrates that bispecific binder modification worked similarly in LNP carrying different cargos.

To demonstrate bispecific binder modification can enhance targeted delivery of LNP carrying different cargos, mRNA coding for GFP and CD19CAR (chimeric antigen receptor specific to CD19), circular RNA coding for CD19CAR, and siRNA against Fluc (siFluc) were synthesized. Both mRNA and circRNA were encapsulated by ALC, M3413 and SM3 formulations, while siRNA was encapsulated by SM102 formulation (FIG. 20).

All of the LNP-RNAs were modified by CD7xPEG binder. hPBMC-T cells were used for GFP and CD19CAR mRNA and circRNA delivery experiments, whereas Jurkat-Fluc cell (Jurkat with firefly luciferase overexpressed, generated by lentiviral transduction) were applied for siRNA delivery. Cell culture and treatment procedures were consistent with description in example 5, and LNP-siRNA was added as 50 ng/well. Indeed, regardless of RNA species, LNP-RNA delivery was significantly boosted by bispecific binder modifications (FIGS. 21 and 22).

Moreover, CAR-T cells generated by binder-LNP-RNA complexes showed potent cytotoxicity activity (FIG. 23). In the cytotoxicity assay, Nalm-6 was used as target cells while T cells and CAR-T cells as effector cells. Target cells were labeled with Cell Explorer™ Live Cell Labeling Kit *Orange (AAT Bioquest) for 30 min. Required labeled target cells and effector cells were then calculated according to indicated E:T ratios and mixed in culture for 6 h. Apoptotic rate was measured by staining of APC-Annexin V and Propidium Iodide (PI) and analyzed by flow cytometry. In brief, Cells that stain positive for Cell Explorer™ are viable cells before mixing with effector cells and will be gated for the apoptosis analysis. Cells stain positive for APC-Annexin V and negative for PI are undergoing apoptosis. Cells that stain positive for both APC-Annexin V and PI are either in the end stage of apoptosis or undergoing necrosis. Cells that stain negative for both APC-Annexin V and PI are alive and not undergoing measurable apoptosis. Apoptotic rate was calculated by number of APC-Annexin V+/PI− plus APC-Annexin V+/PI+ cells divided by number of analyzed cells. Take apoptotic rate when using CAR-T cells as effector for x, and when using T cells for y, the CAR-T killing index was calculated by [(x−y)/(1−y)]*100%.

### Example 8

This example illustrates that accelerated blood clearance (ABC) of LNP-RNA was alleviated by binder modifications

Six-week-old female CD1 mice were ordered from Charles River. Mice were maintained on a 12 hr light cycle with free access to food and water for 96-168 hrs. Mice were dosed 10 mL/kg with 0.01 mg/mL LNP-Fluc (ALC formulation) with and without indicated binder modification. Six hours post dosage, mice were dosed IP with 200 μL of 15 mg/mL D-Luciferin (Revvity) in PBS. Eight minutes post dose, the mice are anesthetized with isoflurane and imaged on an IVIS Spectrum instrument. Regions of interest were defined and Total Flux and Average Radiance were calculated by the Living Image Software. Mice were returned to the holding room and another round of imaging was completed 6 days later. Test articles were re-dosed on study days 7, 14, 21, and 28 and imaged 6 hs post each dose. Images were also recorded on days 6, 13,20,27 and 35 without redosing the test articles. As shown in FIG. 24, compared to naked LNP-Fluc, Fluc activity of multiple binders modified LNP-Fluc were significantly higher over redosing. These results demonstrated that binder modifications alleviate ABC of LNP-RNA.

### Example 9

This example illustrates that different PEG densities and ratios of PEG-lipid all exhibit high delivery effects.

FIG. 25 shows the in vitro data of Non-targeted LNP (black bars) vs CD-5 targeted LNP (blue bars). The 4 plots are at 4 different PEG densities on the LNP surface ranging from 1.5% (standard) to 3%. The increase in the total PEG mole percent was compensated by reducing the mole percent of cholesterol. The general formulation composition for the formulations was as follows: SM 102: DOPE: 18-1 PG: Cholesterol: DSPE-PEG₂ₖ: DMG-PEG₂ₖ at 45:9:10:33-34.5:1.5-3:1.5-3 mole percent. The formulations were dosed to Jurkat E6-1 cells at 100 ng dose for 1E5 cells and incubated for 24 h before reading the eGFP expression profile using ELISA. The total eGFP content was normalized to total protein and plotted. The formulations in the plots are 1: 100% DMG-PEG; 2: 75% DMG-PEG 25% DSPE-PEG; 3: 50% DMG-PEG 50% DSPE-PEG, (in FIG. 25 a c d) and 4: 25% DMG-PEG 75% DSPE-PEG (in FIG. 25 b). The data shows that even with a CD5-targeted system, which is very like the untargeted LNP, any blend of sheddable to non-sheddable PEG containing LNP shows the delivery/expression at all PEG densities. The 3:1 blend of sheddable to non-sheddable PEG containing LNP shows the best delivery/expression at all PEG densities.

### EMBODIMENTS

Embodiment 1. A nanoparticle complex comprising
   (a) a lipid nanoparticle comprising a surface component;
   (b) a bifunctional binder comprising
      (i) a self-binder moiety non-covalently binding to the surface component,
      (ii) a target-binder moiety capable of binding to a targeted cell-surface component of a target cell,
      wherein the self-binder moiety is linked to the target-binder moiety; and
   (c) a cargo carried by the lipid nanoparticle.
Embodiment 2. The nanoparticle complex of Embodiment 1, wherein the cargo is an RNA selected from a group consisting of a circular RNA, a mRNA, a noncoding RNA, a dsRNA, a miRNA, an siRNA and a tRNA; preferably a circular RNA.
Embodiment 3. The nanoparticle complex of Embodiment 1 or 2, wherein the lipid nanoparticle comprises an ionizable lipid, a helper lipid, a PEG-modified lipid, and a cholesterol-based lipid, optionally, the lipid nanoparticle also comprises a biotin-modified lipoid; optionally, the biotin-modified lipoid is biotin-modified cholesterol.
Embodiment 4. The nanoparticle complex of any one of Embodiments 1-3, wherein the ionizable lipid is a cationic lipid selected from the group consisting of HGT4003, HGT5000, HGT5001, HGT5002, DOTMA, DOGS, DOSPA, DOTAP, DODAP, DOTMA, DSDMA, DODMA, DLinDMA, DLenDMA, DODAC, DDAB, DMRIE, CLinDMA, CpLinDMA, DMOBA, DOcarbDAP, DLinDAP, DLincarbDAP, DLinCDAP, DLin-K-XTC2-DMA, DLin-KC2-DMA, AA3-DLinXTC, ALNY-100, NC98-5, C12-200, MC2, MC3, MC4, ALC-0315, SM-102, ATX-001, ATX-100, Lipid 2, Lipid 5, 9A1P9, OF-Deg-Lin, 80-O16B, 93-O17S, 93-O170, 306-O12B, 113-O16B, 306Oi10, 113-O12B, cKK-E12, 98N12-5, OF-02, TLC053, LP01, BAMEA-016B, CL1, BP Lipid 310, Lipid A9, L319, and ICE.
Embodiment 5. The nanoparticle complex of any one of Embodiments 1-4, wherein the helper lipid is a non-cationic lipid selected from the group consisting of DSPC, DOPC, DPPC, DOPG, DPPG, DOPE, POPC, POPE, POPG, DEPE, Egg sphingomyelin, DOPE-mal, DPPE, DMPE, DSPE, 16-O-monomethyl PE, 16-O-dimethyl PE, 18-1-trans PE and SOPE.
Embodiment 6. The nanoparticle complex of any one of Embodiments 1-5, wherein the PEG-modified lipid is a polyethylene glycol (PEG) chain covalently attached to a lipid with alkyl chain, wherein the PEG chain is of 100-20000 Da, optionally, the PEG chain is of 100, 200, 400, 600, 800, 1000, 2000, 4000, 8000, 10000, 20000 Da, optionally, the alkyl chain is of C₆-C₂₀ length.
Embodiment 7. The nanoparticle complex of any one of Embodiments 1-6, wherein the PEG-modified lipid is a glycol (PEG)-modified phospholipid or ceramide, or a combination thereof.
Embodiment 8. The nanoparticle complex of any one of Embodiments 1-7, wherein the PEG-modified lipid is DMG-PEG2000, DSPE-PEG2000 or a combination thereof.
Embodiment 9. The nanoparticle complex of any one of Embodiments 1-8, wherein the cholesterol-based lipid is selected from cholesterol, hydroxy cholesterol and its derivatives, DC-cholesterol HCl, HAPC-Cholesterol, MHAPC-Cholesterol, DMHAPC-Cholesterol, DMPAC-Cholesterol, GL-67, LC-10, PEGylated cholesterol or their derivatives, DC-Choi, 1,4-bis(3-N-oleylamino-propyl) piperazine, ICE, Vit D2, Vit D3, Calcipotriol, Stigmasterol, b-sitosterol, betulin, lupeol, ursolic acid, oleanolic, lipid derivatives of cholesterol lipid where the lipid could be myristate, oleate, nervonate or hemisuccinate
Embodiment 10. The nanoparticle complex of any one of Embodiments 1-9, wherein the lipid nanoparticle comprises
   (1) ALC-0315, cholesterol, DOPE and DMG-PEG2000; or
   (2) ALC-0315, cholesterol, DSPC, DMG-PEG2000 and DSPE-PEG2000; or
   (3) SM102, cholesterol, DOPE, DMG-PEG2000, DSPE-PEG2000 and 18:1PG; or
   (4) Lipid5, cholesterol, DSPC, DMG-PEG2000, DSPE-PEG2000 and 18:1PG; or
   (5) SM102, cholesterol, DSPC and DMG-PEG2000.
Embodiment 11. The nanoparticle complex of any one of Embodiments 1-10, wherein the surface component is an ionizable lipid, a helper lipid, a PEG-modified lipid, or a biotin-modified lipoid; optionally, the biotin-modified lipoid is biotin-modified cholesterol.
Embodiment 12. The nanoparticle complex of any one of Embodiments 1-11, wherein the self-binder moiety is a first antigen binding fragment specifically binding to the surface component.
Embodiment 13. The nanoparticle complex of any one of Embodiments 12, wherein the first antigen binding fragment specifically binds to PEG.
Embodiment 14. The nanoparticle complex of any one of Embodiments 12-13, wherein the first antigen binding fragment specifically binds to the methoxy group of PEG or the backbone of PEG, preferably the first antigen binding fragment specifically binds to the methoxy group of PEG.
Embodiment 15. The nanoparticle complex of any one of Embodiments 12-14, wherein the first antigen binding fragment specifically binds to the cholesterol-based lipid.
Embodiment 16. The nanoparticle complex of any one of Embodiments 3-15, wherein the cholesterol-based lipid is conjugated with a biotin and the first antigen binding fragment specifically binds to the biotin.
Embodiment 17. The nanoparticle complex of any one of Embodiments 12-16, wherein the first antigen binding fragment is derived from an IgG, an IgA, an IgM, an IgE or an IgD.
Embodiment 18. The nanoparticle complex of any one of Embodiments 12-17, wherein the first antigen binding fragment is a Fab, a VHH antibody, an scFv, an scFab, or a diabody.
Embodiment 19. The nanoparticle complex of any one of Embodiments 1-18, wherein the target-binder moiety is an Fc or a fragment thereof of an IgG, an IgA, an IgM, an IgE or an IgD.
Embodiment 20. The nanoparticle complex of any one of Embodiments 1-19, wherein the targeted cell-surface component of the target cell is a protein, a glycoRNA, a lipid or lipid raft.
Embodiment 21. The nanoparticle complex of any one of Embodiments 1-20, wherein the protein is a receptor.
Embodiment 22. The nanoparticle complex of Embodiment 21, wherein the receptor is an Fc receptor, FcgR1, FcgR2, FCgr3, FcgR4, FceR1, FceR2, FcaR1, FcuR, FcdR, or an isoform thereof, or C1qR or FcRn.
Embodiment 23. The nanoparticle complex of any one of Embodiments 1-22, wherein the target cell is an antigen presenting cell, a monocyte, a neutrophil, a macrophage, a dendritic cell, a mast cell, a T cell, a natural killer cell, a Kupffer cell, a B cell, or a tumor cell.
Embodiment 24. The nanoparticle complex of any one of Embodiments 1-23, wherein the target-binder moiety is a second antigen binding fragment specifically binding to the receptor.
Embodiment 25. The nanoparticle complex of Embodiment 24, wherein the second antigen binding fragment is a Fab, a VHH antibody, an scFv, an scFab, or a diabody.
Embodiment 26. The nanoparticle complex of any one of Embodiments 21-25, wherein the receptor is selected from the group consisting of CD2, CD3, CD4, CD5, CD7, CD8, CD25, CD45RA, CD45RO, CTLA4 and PL-L1.
Embodiment 27. The nanoparticle complex of Embodiment 23, wherein the T cell is a CD3+ T cell, a CD4+ T cell, a CD8+ T cell, a T regulatory cell, a cytotoxic T cell, or a helper T cell.
Embodiment 28. The nanoparticle complex of any one of Embodiments 1-27, wherein the self-binder moiety is covalently linked to the target-binder moiety.
Embodiment 29. The nanoparticle complex of any one of Embodiments 1-28, wherein the self-binder moiety is linked to the target-binder moiety via a linker.
Embodiment 30. The nanoparticle complex of Embodiment 29, wherein the linker comprises an Fc or a fragment thereof.
Embodiment 31. The nanoparticle complex of Embodiment 29, wherein the linker comprises a multimerization domain.
Embodiment 32. The nanoparticle complex of any one of Embodiments 1-31, wherein the self-binder moiety is non-covalently linked to the target-binder moiety.
Embodiment 33. The nanoparticle complex of any one of Embodiments 1-32, wherein the nanoparticle complex has at least one of the functions as following, compared to a reference nanoparticle complex:
   (a) substantially avoids uptake by hepatocytes,
   (b) substantially contacts cells displaying the targeted cell-surface component of the target cell, and
   (c) substantially decreases accelerated blood clearance of the nanoparticle complex,
   wherein the reference nanoparticle complex is a nanoparticle complex without the bifunctional binder.
Embodiment 34. A pharmaceutical composition, comprising the nanoparticle complex of any one of Embodiments 1-33 and a pharmaceutically acceptable carrier.
Embodiment 35. The pharmaceutical composition of Embodiment 34, wherein the lipid nanoparticle and the bifunctional binder are packaged in different containers.
Embodiment 36. A method of delivering a cargo molecule to a target cell in a subject, comprising administering to the subject the nanoparticle complex of any one of Embodiments 1-33.
Embodiment 37. An isolated monoclonal antibody or an antigen-binding fragment thereof comprising: a heavy chain (HC) variable region (VH) comprising HC-CDR1, HC-CDR2 and HC-CDR3; and a light chain (LC) variable region (VL) comprising LC-CDR1, LC-CDR2 and LC-CDR3, wherein
   (a) the HC-CDR1, HC-CDR2 and HC-CDR3 sequences are those contained in a VH sequence of SEQ ID NO: 7, and the LC-CDR1, LC-CDR2 and LC-CDR3 sequences are those contained in a VL sequence of SEQ ID NO: 8, or
   (b) the HC-CDR1, HC-CDR2 and HC-CDR3 sequences are those contained in a VH sequence of SEQ ID NO: 15, and the LC-CDR1, LC-CDR2 and LC-CDR3 sequences are those contained in a VL sequence of SEQ ID NO: 16.
Embodiment 38. The isolated monoclonal antibody or an antigen-binding fragment thereof according to Embodiment 38, wherein the HC-CDR1, HC-CDR2, HC-CDR3, LC-CDR1, LC-CDR2, and LC-CDR3 sequences are defined by the Kabat numbering system, the IMGT numbering system, the Chothia numbering system, the Contact numbering system, or the AbM numbering system.
Embodiment 39. The isolated monoclonal antibody or an antigen-binding fragment thereof according to Embodiment 37, wherein
   (a) the HC-CDR1, HC-CDR2 and HC-CDR3 sequences are set forth in SEQ ID NOs: 1-3 respectively; and the LC-CDR1, LC-CDR2 and LC-CDR3 sequences are set forth in SEQ ID NO: 4, GT, and SEQ ID NO: 6 respectively; or
   (b) the HC-CDR1, HC-CDR2 and HC-CDR3 sequences are set forth in SEQ ID NOs: 9-11 respectively; and the LC-CDR1, LC-CDR2 and LC-CDR3 sequences are set forth in SEQ ID NO: 12, GT, and SEQ ID NO: 14 respectively.
Embodiment 40. The isolated monoclonal antibody or an antigen-binding fragment thereof according to Embodiment 39, wherein
   (a) the VH comprises a sequence having at least 80% identity to SEQ ID NO: 7, and the VL comprises a sequence having at least 80% identity to SEQ ID NO: 8; or
   (b) the VH comprises a sequence having at least 80% identity to SEQ ID NO: 15, and the VL comprises a sequence having at least 80% identity to SEQ ID NO: 16.
Embodiment 41. A bifunctional binder comprising:
   (a) a self-binder moiety capable of binding to a surface component of a nanoparticle;
   (b) a target-binder moiety capable of binding to a targeted cell-surface component of a target cell.
Embodiment 42. The bifunctional binder of Embodiment 41, wherein the surface component of the nanoparticle is an ionizable lipid, a helper lipid, a PEG-modified lipid, or a cholesterol-based lipid.
Embodiment 43. The bifunctional binder of Embodiment 42, wherein the self-binder moiety is a first antigen binding fragment specifically binding to the surface component.
Embodiment 44. The bifunctional binder of Embodiment 43, wherein the first antigen binding fragment specifically binds to PEG.
Embodiment 44. The bifunctional binder of Embodiment 42, wherein the first antigen binding fragment specifically binds to the cholesterol-based lipid.
Embodiment 45. The bifunctional binder of Embodiment 44, wherein the cholesterol-based lipid is conjugated with a biotin and the first antigen binding fragment specifically binds to the biotin.
Embodiment 46. The bifunctional binder of any one of Embodiments 41-45, wherein the target cell is an antigen presenting cell, a monocyte, a neutrophil, a macrophage, a dendritic cell, a mast cell, a T cell, a natural killer cell, a Kupffer cell, a B cell, or a tumor cell.
Embodiment 47. The bifunctional binder of any one of Embodiments 41-46, wherein the T cell is a CD3+ T cell, a CD4+ T cell, a CD8+ T cell, a T regulatory cell, a cytotoxic T cell, or a helper T cell.
Embodiment 48. The bifunctional binder of any one of Embodiments 41-47, wherein the target-binder moiety is a second antigen binding fragment specifically binding to the receptor.
Embodiment 49. The bifunctional binder of Embodiment 48, wherein the receptor is selected from the group consisting of CD2, CD3, CD4, CD5, CD7, CD8, CD25, CD45RA, CD45RO, CTLA4 and PD-L1.
Embodiment 50. The bifunctional binder of any one of Embodiments 48-49, wherein the second antigen binding fragment is a Fab, a VHH antibody, an scFv, an scFab, or a diabody.
Embodiment 51. The bifunctional binder of any one of Embodiments 41-50, wherein the self-binder moiety is covalently or non-covalently linked to the target-binder moiety.
Embodiment 52. The bifunctional binder of Embodiment 51, wherein the self-binder moiety is linked to the target-binder moiety via a linker.
Embodiment 53. The bifunctional binder of any one of Embodiments 41-52, wherein the bi-functional binder has a structure of
   (a) [P-VH]-L1-[P-VL]-L2-[T-VHH]; or
   (b) [P-VH]-L1-[P-VL]-L2-[T-VH]-L3-[T-VL]; or
   (c) a dimer of a first chain and a second chain, wherein the first chain has a structure of [P-VL]-CL, and the second chain has a structure of [P-VH]-CH1-Hinge-CH2-CH3-L2-[T-VHH]; or
   (d) a dimer of a first chain and a second chain, wherein the first chain has a structure of [P-VL]-CL, and the second chain has a structure of [P-VH]-CH1-Hinge-CH2-CH3-L2-[T-VH]-L3-[T-VL];
   wherein
   P-VL and P-VH refer to VL and VH of self-binder moiety, respectively,
   T-VL, T-VH and T-VHH refer VL, VH and VHH of target-binder moiety, respectively, and
   L1, L2, L3 are independently a peptide linker.
Embodiment 54. The bifunctional binder of Embodiment 53, wherein in structures (a), (b), (c), or (d), P-VH comprises HC-CDR1, HC-CDR2 and HC-CDR3; P-VL comprises LC-CDR1, LC-CDR2 and LC-CDR3,
   the HC-CDR1, HC-CDR2 and HC-CDR3 sequences are those contained in a VH sequence of SEQ ID NO:7, SEQ ID NO: 15 or SEQ ID NO: 22; the LC-CDR1, LC-CDR2 and LC-CDR3 sequences are those contained in a VL sequence of SEQ ID NO: 8, SEQ ID NO:16 or SEQ ID NO:23.
Embodiment 55. The bifunctional binder of Embodiment 54, wherein
   (i) the HC-CDR1, HC-CDR2 and HC-CDR3 sequences are set forth in SEQ ID NOs 1-3 respectively; and the LC-CDR1, LC-CDR2 and LC-CDR3 sequences are set forth in SEQ ID NO: 4, GT, and SEQ ID NO: 6 respectively,
   (ii) the HC-CDR1, HC-CDR2 and HC-CDR3 sequences are set forth in SEQ ID NOs 9-11 respectively; and the LC-CDR1, LC-CDR2 and LC-CDR3 sequences are set forth in SEQ ID NO: 12, GT, and SEQ ID NO: 14 respectively, or
   (iii) the HC-CDR1, HC-CDR2 and HC-CDR3 sequences are set forth in SEQ ID NOs 52-54 respectively; and the LC-CDR1, LC-CDR2 and LC-CDR3 sequences are set forth in SEQ ID NO: 55, WA, and SEQ ID NO: 57 respectively.
Embodiment 56. The bifunctional binder of Embodiment 55, wherein in structures (a), (b), (c), or (d),
   (i) P-VH comprises a sequence having at least 80% identity to SEQ ID NO: 7, and P-VL comprises a sequence having at least 80% identity to SEQ ID NO: 8;
   (ii) P-VH comprises a sequence having at least 80% identity to SEQ ID NO: 15, and P-VL comprises a sequence having at least 80% identity to SEQ ID NO: 16; or
   (iii) P-VH comprises a sequence having at least 80% identity to SEQ ID NO: 22, and P-VL comprises a sequence having at least 80% identity to SEQ ID NO: 23.
Embodiment 57. The bifunctional binder of any one of Embodiments 53-56, wherein in structures (a), or (c), T-VHH comprises HC-CDR1, HC-CDR2 and HC-CDR3, the HC-CDR1, HC-CDR2 and HC-CDR3 sequences are those contained in a sequence of SEQ ID NO: 21 or SEQ ID NO: 24.
Embodiment 58. The bifunctional binder of any one of Embodiments 53-57, wherein
   (i) the HC-CDR1, HC-CDR2 and HC-CDR3 sequences of T-VHH are set forth in SEQ ID NOs 58-60 respectively; or
   (ii) the HC-CDR1, HC-CDR2 and HC-CDR3 sequences of T-VHH are set forth in SEQ ID NOs 61-63 respectively.
Embodiment 59. The bifunctional binder of any one of Embodiments 53-58, wherein T-VHH comprises a sequence having at least 80% identity to SEQ ID NO: 21 or SEQ ID NO: 24.
Embodiment 60. The bifunctional binder of any one of Embodiments 53-59, wherein in structures (b) or (d),
   T-VH comprises HC-CDR1, HC-CDR2 and HC-CDR3; T-VL comprises LC-CDR1, LC-CDR2 and LC-CDR3,
   the HC-CDR1, HC-CDR2 and HC-CDR3 of T-VH sequences are those contained in a VH sequence of SEQ ID NO: 17, or SEQ ID NO: 19; the LC-CDR1, LC-CDR2 and LC-CDR3 of T-VL sequences are those contained in a VL sequence of SEQ ID NO: 18 or SEQ ID NO: 20.
Embodiment 61. The bifunctional binder of Embodiment 60, wherein
   (i) the HC-CDR1, HC-CDR2 and HC-CDR3 of T-VH sequences are set forth in SEQ ID NOs 64-66 respectively; and the LC-CDR1, LC-CDR2 and LC-CDR3 of T-VL sequences are set forth in SEQ ID NO: 67, GN, and SEQ ID NO: 69 respectively; or
   (ii) the HC-CDR1, HC-CDR2 and HC-CDR3 of T-VH sequences are set forth in SEQ ID NOs 70-72 respectively; and the LC-CDR1, LC-CDR2 and LC-CDR3 of T-VL sequences are set forth in SEQ ID NO: 73, YT, and SEQ ID NO: 75 respectively.
Embodiment 62. The bifunctional binder of any one of Embodiments 53-61, wherein in structures (b), or (d),
   (i) T-VH comprises a sequence having at least 80% identity to SEQ ID NO:17, and T-VL comprises a sequence having at least 80% identity to SEQ ID NO:18; or
   (ii) T-VH comprises a sequence having at least 80% identity to SEQ ID NO:19, and T-VL comprises a sequence having at least 80% identity to SEQ ID NO:20.
Embodiment 63. The bifunctional binder of any one of Embodiments 53-62, wherein the peptide linker has a length of about 2 to 200 amino acids; optionally, about 2 to 100 amino acids; optionally, about 2 to 50 amino acids, optionally, about 2 to 30 amino acids;
   optionally, the peptide linker includes a hydrophilic amino acid;
   optionally, the peptide linker includes amino acid glycine (G) and/or serine (S);
   optionally, the peptide linker includes a sequence of (GS)ₙ, (GGS)ₙ, (GSGGS)ₙ, or (GₙS)ₘ, wherein each of n and m is 1 to 10.

Embodiment 64. The bifunctional binder of any one of Embodiments 53-63, wherein L1, L2, L3 are independently a peptide linker having a sequence of GGGGS (SEQ ID NO:76), GGGGSGGGGSGGGGS (SEQ ID NO:77), or GGGGSGGGGSGGGGS (SEQ ID NO:78).

Embodiment 65. The bifunctional binder of any one of Embodiments 53-64, wherein in structures (c) or (d), CL, CH1, Hinge, CH2, or CH3 is derived from an IgG1, an IgG2, an IgG3, an IgG4, an IgA1, an IgA2, an IgM1, an IgM2, an IgE or an IgD.

Embodiment 66. The bifunctional binder of any one of Embodiments 53-65, wherein the bifunctional binder has (a) structure having a sequence at least 80% identity to SEQ ID NO: 51.

Embodiment 67. The bifunctional binder of any one of Embodiments 53-65, wherein the bifunctional binder has (b) structure having a sequence at least 80% identity to SEQ ID NO: 40.

Embodiment 68. The bifunctional binder of any one of Embodiments 53-65, wherein the bifunctional binder has (c) structure, the first chain has a sequence at least 80% identity to SEQ ID NO:49, the second chain has a sequence at least 80% identity to SEQ ID NO:50.

Embodiment 69. The bifunctional binder of any one of Embodiments 53-65, wherein the bifunctional binder has (c) structure, the first chain has a sequence at least 80% identity to SEQ ID NO:44, the second chain has a sequence at least 80% identity to SEQ ID NO:45.

Embodiment 70. The bifunctional binder of any one of Embodiments 53-65, wherein the bifunctional binder has (c) structure, the first chain has a sequence at least 80% identity to SEQ ID NO:46, the second chain has a sequence at least 80% identity to SEQ ID NO:47.

Embodiment 71. The bifunctional binder of any one of Embodiments 53-65, wherein the bifunctional binder has (d) structure, the first chain has a sequence at least 80% identity to SEQ ID NO:41, the second chain has a sequence at least 80% identity to SEQ ID NO:42.

Embodiment 72. The bifunctional binder of any one of Embodiments 53-65, wherein the bifunctional binder has (d) structure, the first chain has a sequence at least 80% identity to SEQ ID NO:38, the second chain has a sequence at least 80% identity to SEQ ID NO:39.

Embodiment 73. The bifunctional binder of any one of Embodiments 53-65, wherein the bifunctional binder has (d) structure, the first chain has a sequence at least 80% identity to SEQ ID NO:27, the second chain has a sequence at least 80% identity to SEQ ID NO:28.

Embodiment 74. The bifunctional binder of any one of Embodiments 53-65, wherein the bifunctional binder has (d) structure, the first chain has a sequence at least 80% identity to SEQ ID NO:29, the second chain has a sequence at least 80% identity to SEQ ID NO:30.

1-23. (canceled)

24. An in vivo method of delivering a cargo molecule to a target cell in a subject, comprising administering to the subject an effective amount of a nanoparticle complex comprising:
(a) a lipid nanoparticle comprising a surface component, wherein the lipid nanoparticle comprises an ionizable lipid, a helper lipid, a PEG-modified lipid, and a cholesterol-based lipid, wherein the surface component is the PEG-modified lipid;
(b) a bifunctional binder comprising:
(i) a self-binder moiety non-covalently binding to the surface component; and
(ii) a target-binder moiety capable of binding to a targeted cell-surface component of a target cell,
wherein the self-binder moiety is linked to the target-binder moiety and the self-binder moiety is a first antigen binding fragment specifically binding to the surface component, and wherein the first antigen binding fragment specifically binds to PEG; and
(c) a cargo carried by the lipid nanoparticle.

25. The method of claim 24, wherein the cargo is one or more nucleic acid molecules.

26. The method of claim 24, wherein the cargo is a DNA or an RNA.

27. The method of claim 26, wherein the cargo is selected from the group consisting of a circular RNA, a mRNA, a noncoding RNA, a dsRNA, a miRNA, an siRNA and a tRNA.

28. The method of claim 24, wherein the ionizable lipid is a cationic lipid selected from the group consisting of HGT4003, HGT5000, HGT5001, HGT5002, DOTMA, DOGS, DOSPA, DOTAP, DODAP, DOTMA, DSDMA, DODMA, DLinDMA, DLenDMA, DODAC, DDAB, DMRIE, CLinDMA, CpLinDMA, DMOBA, DOcarbDAP, DLinDAP, DLincarbDAP, DLinCDAP, DLin-K-XTC2-DMA, DLin-KC2-DMA, AA3-DLinXTC, ALNY-100, NC98-5, C12-200, MC2, MC3, MC4, ALC-0315, SM-102, ATX-001, ATX-100, Lipid 2, Lipid 5, 9A1P9, OF-Deg-Lin, 80-O16B, 93-O17S, 93-O17O, 306-O12B, 113-O16B, 306Oi10, 113-O12B, cKK-E12, 98N12-5, OF-02, TLC053, LP01, BAMEA-O16B, CL1, BP Lipid 310, Lipid A9, L319, and ICE.

29. The method of claim 24, wherein the helper lipid is a non-cationic lipid selected from the group consisting of DSPC, DOPC, DPPC, DOPG, DPPG, DOPE, POPC, POPE, POPG, DEPE, Egg sphingomyelin, DOPE-mal, DPPE, DMPE, DSPE, 16-O-monomethyl PE, 16-O-dimethyl PE, 18-1-trans PE and SOPE.

30. The method of claim 24, wherein the PEG-modified lipid is a polyethylene glycol (PEG) chain covalently attached to a lipid with alkyl chain of C₆-C₂₀ length, wherein the PEG chain is of 100-20000 Da.

31. The method of claim 24, wherein the PEG-modified lipid is a glycol (PEG)-modified phospholipid or ceramide, or a combination thereof.

32. The method of claim 31, wherein the PEG-modified lipid is DMG-PEG2000, DSPE-PEG2000 or a combination thereof.

33. The method of claim 24, wherein the cholesterol-based lipid is selected from cholesterol, hydroxy cholesterol and its derivatives, DC-cholesterol HCl, HAPC-Cholesterol, MHAPC-Cholesterol, DMHAPC-Cholesterol, DMPAC-Cholesterol, GL-67, LC-10, PEGylated cholesterol or their derivatives, DC-Choi, 1,4-bis(3-N-oleylamino-propyl) piperazine, ICE, Vit D2, Vit D3, Calcipotriol, Stigmasterol, b-sitosterol, betulin, lupeol, ursolic acid, oleanolic, lipid derivatives of cholesterol lipid where the lipid could be myristate, oleate, nervonate or hemisuccinate.

34. The method of claim 24, wherein the lipid nanoparticle comprises:
(1) an ALC-0315, cholesterol, DOPE and DMG-PEG2000; or
(2) an ALC-0315, cholesterol, DSPC, DMG-PEG2000 and DSPE-PEG2000;
or (3) a SM102, cholesterol, DOPE, DMG-PEG2000, DSPE-PEG2000 and 18:1PG; or
(4) a Lipid5, cholesterol, DSPC, DMG-PEG2000, DSPE-PEG2000 and 18:1PG; or
(5) a SM102, cholesterol, DSPC and DMG-PEG2000.

35. The method of claim 24, wherein the first antigen binding fragment specifically binds to the methoxy group of PEG or the backbone of PEG.

36. The method of claim 35, wherein the first antigen binding fragment is derived from an IgG, an IgA, an IgM, an IgE or an IgD.

37. The method of claim 24, wherein the first antigen binding fragment is a Fab, a VHH antibody, an scFv, an scFab, or a diabody.

38. The method of claim 24, wherein the target-binder moiety is an Fc or a fragment thereof of an IgG, an IgA, an IgM, an IgE or an IgD.

39. The method of claim 24, wherein the targeted cell-surface component of the target cell is a protein, a glycoRNA, a lipid or lipid raft.

40. The method of claim 39, wherein the protein is a receptor selected from the group consisting of an Fc receptor, FcgR1, FcgR2, FCgr3, FcgR4, FceR1, FceR2, FcaR1, FcuR, FcdR, or an isoform thereof, C1qR and FcRn.

41. The method of claim 24, wherein the protein is a receptor selected from the group consisting of CD2, CD3, CD4, CD5, CD7, CD8, CD25, CD45RA, CD45RO, CTLA4 and PD-L1.

42. The method of claim 24, wherein the target cell is an antigen presenting cell, a monocyte, a neutrophil, a macrophage, a dendritic cell, a mast cell, a T cell, a natural killer cell, a Kupffer cell, a B cell or a tumor cell.

43. The method of claim 42, wherein the T cell is a CD3+ T cell, a CD4+ T cell, a CD8+ T cell, a T regulatory cell, a cytotoxic T cell, or a helper T cell.

44. The method of claim 24, wherein the target-binder moiety is a second antigen binding fragment specifically binding to the receptor.

45. The method of claim 44, wherein the second antigen binding fragment is a Fab, a VHH antibody, an scFv, an scFab, or a diabody.

46. The method of claim 24, wherein the self-binder moiety is covalently linked to the target-binder moiety.

47. The method of claim 46, wherein the self-binder moiety is linked to the target-binder moiety via a linker.

48. The method of claim 47, wherein the linker comprises an Fc or a fragment thereof.

49. The method of claim 48, wherein the linker comprises a multimerization domain.

50. The method of claim 24, wherein the self-binder moiety is non-covalently linked to the target-binder moiety.

51. The method of claim 24, wherein the nanoparticle complex has at least one of the functions as following, compared to a reference nanoparticle complex:
(a) substantially avoids or reduces uptake by hepatocytes,
(b) substantially contacts cells displaying the targeted cell-surface component of the target cell, and
(c) substantially decreases accelerated blood clearance of the nanoparticle complex, wherein the reference nanoparticle complex is a nanoparticle complex without the bifunctional binder.

52. The method of claim 24, wherein the target cell is not hepatocyte.


SEQUENCE LISTING (78 sequences)

SEQ ID NO: 1 - protein, 8 aa, unidentified
GFTFSDYG

SEQ ID NO: 2 - protein, 8 aa, unidentified
ISSGSSTI

SEQ ID NO: 3 - protein, 12 aa, unidentified
VRAEITTVVADY

SEQ ID NO: 4 - protein, 9 aa, unidentified
TGAVTTSNY

SEQ ID NO: 5 - skipped

SEQ ID NO: 6 - protein, 9 aa, unidentified
VLWYSNHWV

SEQ ID NO: 7 - protein, 119 aa, unidentified
EVQLVESGGGLVKPGGSLKLSCAASGFTFSDYGMHWVRQAPEKGLEWVAYISSGSSTIYYADTVKGRFTISRDNAKNTLFLQMTNLRSEDTAMYYCVRAEITTVVADYWGQGTTLTVSS

SEQ ID NO: 8 - protein, 109 aa, unidentified
QAVVTQESALTTSPGETVTLTCRLSTGAVTTSNYANWVQEKPDHLFTGLIGGTNNRAPGVPARFSGSLIGDKAALTITGAQTEDEARYFCVLWYSNHWVFGGGTKLTVL

SEQ ID NO: 9 - protein, 9 aa, unidentified
GYSITSGYY

SEQ ID NO: 10 - protein, 7 aa, unidentified
ISYDGSN

SEQ ID NO: 11 - protein, 12 aa, unidentified
ASGNGYDVGFAY

SEQ ID NO: 12 - protein, 9 aa, unidentified
TGAVTTSNY

SEQ ID NO: 13 - skipped

SEQ ID NO: 14 - protein, 9 aa, unidentified
ALWYSTHYV

SEQ ID NO: 15 - protein, 119 aa, unidentified
DVQLQESGPGLVKPSQSLSLTCSVTGYSITSGYYWNWVRQFPGNKLEWMGYISYDGSNNYKPSLKNRISITRDTSNNQFFLKLNSVTAEDTATYYCASGNGYDVGFAYWGQGTLVTVSA

SEQ ID NO: 16 - protein, 109 aa, unidentified
QAVVTQESALTTSPGGTVILTCRSSTGAVTTSNYANWVQEKPDHLFTGLIGGTSNRTPGVPVRFSGSLIGDKAALTITGAQTEDDAMYFCALWYSTHYVFGGGTKVTVL

SEQ ID NO: 17 - protein, 118 aa, unidentified
EVQLLESGGGLVKPGGSLRLSCAASGFTFNNYTMNWVRQAPGKGLEWVSSISSSSSYIYYADSVKGRFTISRDNAKNSLFLQMNSLRGEDTAVYYCARYFSGSAGDYWGQGTLVTVSS

SEQ ID NO: 18 - protein, 112 aa, unidentified
QSVVTQPPSVSGAPGQRVTISCTGSSSNIGAGYDVHWYQQLPGTAPKLLIYGNINRPSGVPDRFSGSKSGTSASLAITGLQAEDEADYYCGTWDNSLSAHYVFGTGTKVTVL

SEQ ID NO: 19 - protein, 117 aa, unidentified
EVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSS

SEQ ID NO: 20 - protein, 107 aa, unidentified
DIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIK

SEQ ID NO: 21 - protein, 125 aa, unidentified
EVQLVESGGGLVQPGGSLRLSCAASGFTFDDYAIGWFRQAPGKGREGVACIRIFDRHTYYADSVKGRFTISSDNSKNTVYLQMNSLRAEDTATYYCAAGSFWGCTRPEGDMDYFGQGTLVQVQSA

SEQ ID NO: 22 - protein, 133 aa, unidentified
MGWSCIILFLVATATGVHSQVQLVQSGSELKKPGASVKVSCKASGYTFKNYGMNWVRQAPGQGLEWMGWINTYTGQPIYANDFKGRFVFSLDTSVSTAYLQISSLKAEDTAVYYCARDWGPYWGQGTLVTVSS

SEQ ID NO: 23 - protein, 112 aa, unidentified
DIVMTQSPDSLAVSLGERATINCKSSQSVLYSSNQMNYLAWYQQKPGQPPKLLIYWASTRESGVPDRFSGSGSGTDFTLTISSLQAEDVAVYYCLQYLSSWTFGGGTKLEIK

SEQ ID NO: 24 - protein, 125 aa, unidentified
QVQLVESGGGLVQPGGSLRLSCAASGRTFISYALGWFRQAPGQGLEAVAAISWSGSSTYYADSVKGRFTISRDNSKNTLYLQMNSLRAEDTAVYYCAADRTAATGSNLLRKYDYWGQGTLVTVSS

SEQ ID NO: 25 - protein, 240 aa, unidentified
ATMGWSCIILFLVATATGVHSQSVVTQPPSVSGAPGQRVTISCTGSSSNIGAGYDVHWYQQLPGTAPKLLIYGNINRPSGVPDRFSGSKSGTSASLAITGLQAEDEADYYCGTWDNSLSAHYVFGTGTKVTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 448 aa, unidentified
EVQLLESGGGLVKPGGSLRLSCAASGFTFNNYTMNWVRQAPGKGLEWVSSISSSSSYIYYADSVKGRFTISRDNAKNSLFLQMNSLRGEDTAVYYCARYFSGSAGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 215 aa, unidentified
QAVVTQESALTTSPGETVTLTCRLSTGAVTTSNYANWVQEKPDHLFTGLIGGTNNRAPGVPARFSGSLIGDKAALTITGAQTEDEARYFCVLWYSNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 28 - protein, 714 aa, unidentified
EVQLVESGGGLVKPGGSLKLSCAASGFTFSDYGMHWVRQAPEKGLEWVAYISSGSSTIYYADTVKGRFTISRDNAKNTLFLQMTNLRSEDTAMYYCVRAEITTVVADYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQSVVTQPPSVSGAPGQRVTISCTGSSSNIGAGYDVHWYQQLPGTAPKLLIYGNINRPSGVPDRFSGSKSGTSASLAITGLQAEDEADYYCGTWDNSLSAHYVFGTGTKVTVLGGGGSGGGGSGGGGSEVQLLESGGGLVKPGGSLRLSCAASGFTFNNYTMNWVRQAPGKGLEWVSSISSSSSYIYYADSVKGRFTISRDNAKNSLFLQMNSLRGEDTAVYYCARYFSGSAGDYWGQGTLVTVSSGGGGS

SEQ ID NO: 29 - protein, 215 aa, unidentified
QAVVTQESALTTSPGGTVILTCRSSTGAVTTSNYANWVQEKPDHLFTGLIGGTSNRTPGVPVRFSGSLIGDKAALTITGAQTEDDAMYFCALWYSTHYVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 30 - protein, 709 aa, unidentified
DVQLQESGPGLVKPSQSLSLTCSVTGYSITSGYYWNWVRQFPGNKLEWMGYISYDGSNNYKPSLKNRISITRDTSNNQFFLKLNSVTAEDTATYYCASGNGYDVGFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQSVVTQPPSVSGAPGQRVTISCTGSSSNIGAGYDVHWYQQLPGTAPKLLIYGNINRPSGVPDRFSGSKSGTSASLAITGLQAEDEADYYCGTWDNSLSAHYVFGTGTKVTVLGGGGSGGGGSGGGGSEVQLLESGGGLVKPGGSLRLSCAASGFTFNNYTMNWVRQAPGKGLEWVSSISSSSSYIYYADSVKGRFTISRDNAKNSLFLQMNSLRGEDTAVYYCARYFSGSAGDYWGQGTLVTVSS

SEQ ID NO: 31 - protein, 215 aa, unidentified; identical to SEQ ID NO: 27

SEQ ID NO: 32 - protein, 717 aa, unidentified
EVQLVESGGGLVKPGGSLKLSCAASGFTFSDYGMHWVRQAPEKGLEWVAYISSGSSTIYYADTVKGRFTISRDNAKNTLFLQMTNLRSEDTAMYYCVRAEITTVVADYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDVVMTQTPLSLPVSLGDQASISCRSSQSLVHSNGNTYLRWYLQKPGQSPKVLIYKVSNRFSGVPDRFSGSGSGTDFTLKISRVEAEDLGVYFCSQSTHVPWTFGGGTKLEIKGGGGSGGGGSGGGGSGGGGSEVKLDETGGGLVQPGRPMKLSCVASGFTFSDYWMNWVRQSPEKGLEWVAQIRNKPYNYETYYSDSVKGRFTISRDDSKSSVYLQMNNLRVEDMGIYYCTGSYYGMDYWGQGTSVTVSGGGGS

SEQ ID NO: 33 - protein, 513 aa, unidentified
EVQLVESGGGLVKPGGSLKLSCAASGFTFSDYGMHWVRQAPEKGLEWVAYISSGSSTIYYADTVKGRFTISRDNAKNTLFLQMTNLRSEDTAMYYCVRAEITTVVADYWGQGTTLTVSSGGGGSGGGGSGGGGSQAVVTQESALTTSPGETVTLTCRLSTGAVTTSNYANWVQEKPDHLFTGLIGGTNNRAPGVPARFSGSLIGDKAALTITGAQTEDEARYFCVLWYSNHWVFGGGTKLTVLSGGGGSDVVMTQTPLSLPVSLGDQASISCRSSQSLVHSNGNTYLRWYLQKPGQSPKVLIYKVSNRFSGVPDRFSGSGSGTDFTLKISRVEAEDLGVYFCSQSTHVPWTFGGGTKLEIKGGGGSGGGGSGGGGSGGGGSEVKLDETGGGLVQPGRPMKLSCVASGFTFSDYWMNWVRQSPEKGLEWVAQIRNKPYNYETYYSDSVKGRFTISRDDSKSSVYLQMNNLRVEDMGIYYCTGSYYGMDYWGQGTSVTVSWGQGTSVTVSGGGGS

SEQ ID NO: 34 - protein, 215 aa, unidentified; identical to SEQ ID NO: 29

SEQ ID NO: 35 - protein, 718 aa, unidentified
DVQLQESGPGLVKPSQSLSLTCSVTGYSITSGYYWNWVRQFPGNKLEWMGYISYDGSNNYKPSLKNRISITRDTSNNQFFLKLNSVTAEDTATYYCASGNGYDVGFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSDVVMTQTPLSLPVSLGDQASISCRSSQSLVHSNGNTYLRWYLQKPGQSPKVLIYKVSNRFSGVPDRFSGSGSGTDFTLKISRVEAEDLGVYFCSQSTHVPWTFGGGTKLEIKGGGGSGGGGSGGGGSGGGGSEVKLDETGGGLVQPGRPMKLSCVASGFTFSDYWMNWVRQSPEKGLEWVAQIRNKPYNYETYYSDSVKGRFTISRDDSKSSVYLQMNNLRVEDMGIYYCTGSYYGMDYWGQGTSVTVSGGGGS

SEQ ID NO: 36 - protein, 214 aa, unidentified
DIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 447 aa, unidentified
EVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 215 aa, unidentified; identical to SEQ ID NO: 27

SEQ ID NO: 39 - protein, 712 aa, unidentified
EVQLVESGGGLVKPGGSLKLSCAASGFTFSDYGMHWVRQAPEKGLEWVAYISSGSSTIYYADTVKGRFTISRDNAKNTLFLQMTNLRSEDTAMYYCVRAEITTVVADYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSGGGGS

SEQ ID NO: 40 - protein, 488 aa, unidentified
EVQLVESGGGLVKPGGSLKLSCAASGFTFSDYGMHWVRQAPEKGLEWVAYISSGSSTIYYADTVKGRFTISRDNAKNTLFLQMTNLRSEDTAMYYCVRAEITTVVADYWGQGTTLTVSSGGGGSGGGGSGGGGSQAVVTQESALTTSPGETVTLTCRLSTGAVTTSNYANWVQEKPDHLFTGLIGGTNNRAPGVPARFSGSLIGDKAALTITGAQTEDEARYFCVLWYSNHWVFGGGTKLTVLSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSGGGGSGGGGSGGGGSDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIK

SEQ ID NO: 41 - protein, 215 aa, unidentified; identical to SEQ ID NO: 29

SEQ ID NO: 42 - protein, 708 aa, unidentified
DVQLQESGPGLVKPSQSLSLTCSVTGYSITSGYYWNWVRQFPGNKLEWMGYISYDGSNNYKPSLKNRISITRDTSNNQFFLKLNSVTAEDTATYYCASGNGYDVGFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSS

SEQ ID NO: 43 - protein, 357 aa, unidentified
EVQLVESGGGLVQPGGSLRLSCAASGFTFDDYAIGWFRQAPGKGREGVACIRIFDRHTYYADSVKGRFTISSDNSKNTVYLQMNSLRAEDTATYYCAAGSFWGCTRPEGDMDYFGQGTLVQVQSAEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 44 - protein, 215 aa, unidentified; identical to SEQ ID NO: 27

SEQ ID NO: 45 - protein, 589 aa, unidentified
EVQLVESGGGLVKPGGSLKLSCAASGFTFSDYGMHWVRQAPEKGLEWVAYISSGSSTIYYADTVKGRFTISRDNAKNTLFLQMTNLRSEDTAMYYCVRAEITTVVADYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTFDDYAIGWFRQAPGKGREGVACIRIFDRHTYYADSVKGRFTISSDNSKNTVYLQMNSLRAEDTATYYCAAGSFWGCTRPEGDMDYFGQGTLVQVQSA

SEQ ID NO: 46 - protein, 215 aa, unidentified; identical to SEQ ID NO: 29

SEQ ID NO: 47 - protein, 589 aa, unidentified
DVQLQESGPGLVKPSQSLSLTCSVTGYSITSGYYWNWVRQFPGNKLEWMGYISYDGSNNYKPSLKNRISITRDTSNNQFFLKLNSVTAEDTATYYCASGNGYDVGFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTFDDYAIGWFRQAPGKGREGVACIRIFDRHTYYADSVKGRFTISSDNSKNTVYLQMNSLRAEDTATYYCAAGSFWGCTRPEGDMDYFGQGTLVQVQSA

SEQ ID NO: 48 - protein, 352 aa, unidentified
QVQLVESGGGLVQPGGSLRLSCAASGRTFISYALGWFRQAPGQGLEAVAAISWSGSSTYYADSVKGRFTISRDNSKNTLYLQMNSLRAEDTAVYYCAADRTAATGSNLLRKYDYWGQGTLVTVSSVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 49 - protein, 219 aa, unidentified
DIVMTQSPDSLAVSLGERATINCKSSQSVLYSSNQMNYLAWYQQKPGQPPKLLIYWASTRESGVPDRFSGSGSGTDFTLTISSLQAEDVAVYYCLQYLSSWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 584 aa, unidentified
QVQLVQSGSELKKPGASVKVSCKASGYTFKNYGMNWVRQAPGQGLEWMGWINTYTGQPIYANDFKGRFVFSLDTSVSTAYLQISSLKAEDTAVYYCARDWGPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLVESGGGLVQPGGSLRLSCAASGRTFISYALGWFRQAPGQGLEAVAAISWSGSSTYYADSVKGRFTISRDNSKNTLYLQMNSLRAEDTAVYYCAADRTAATGSNLLRKYDYWGQGTLVTVSS

SEQ ID NO: 51 - protein, 400 aa, unidentified
MGWSCIILFLVATATGVHSQVQLVQSGSELKKPGASVKVSCKASGYTFKNYGMNWVRQAPGQGLEWMGWINTYTGQPIYANDFKGRFVFSLDTSVSTAYLQISSLKAEDTAVYYCARDWGPYWGQGTLVTVSSGGGGSGGGGSGGGGSDIVMTQSPDSLAVSLGERATINCKSSQSVLYSSNQMNYLAWYQQKPGQPPKLLIYWASTRESGVPDRFSGSGSGTDFTLTISSLQAEDVAVYYCLQYLSSWTFGGGTKLEIKGGGGSGGGGSGGGGSQVQLVESGGGLVQPGGSLRLSCAASGRTFISYALGWFRQAPGQGLEAVAAISWSGSSTYYADSVKGRFTISRDNSKNTLYLQMNSLRAEDTAVYYCAADRTAATGSNLLRKYDYWGQGTLVTVSS

SEQ ID NO: 52 - protein, 8 aa, unidentified
GYTFKNYG

SEQ ID NO: 53 - protein, 8 aa, unidentified
INTYTGQP

SEQ ID NO: 54 - protein, 7 aa, unidentified
ARDWGPY

SEQ ID NO: 55 - protein, 12 aa, unidentified
QSVLYSSNQMNY

SEQ ID NO: 56 - skipped

SEQ ID NO: 57 - protein, 8 aa, unidentified
LQYLSSWT

SEQ ID NO: 58 - protein, 8 aa, unidentified
GFTFDDYA

SEQ ID NO: 59 - protein, 8 aa, unidentified
IRIFDRHT

SEQ ID NO: 60 - protein, 17 aa, unidentified
AAGSFWGCTRPEGDMDY

SEQ ID NO: 61 - protein, 8 aa, unidentified
GRTFISYA

SEQ ID NO: 62 - protein, 8 aa, unidentified
ISWSGSST

SEQ ID NO: 63 - protein, 18 aa, unidentified
AADRTAATGSNLLRKYDY

SEQ ID NO: 64 - protein, 8 aa, unidentified
GFTFNNYT

SEQ ID NO: 65 - protein, 8 aa, unidentified
ISSSSSYI

SEQ ID NO: 66 - protein, 11 aa, unidentified
ARYFSGSAGDY

SEQ ID NO: 67 - protein, 9 aa, unidentified
SSNIGAGYD

SEQ ID NO: 68 - skipped

SEQ ID NO: 69 - protein, 12 aa, unidentified
GTWDNSLSAHYV

SEQ ID NO: 70 - protein, 8 aa, unidentified
GLTFSSYA

SEQ ID NO: 71 - protein, 7 aa, unidentified
ISSGGFT

SEQ ID NO: 72 - protein, 11 aa, unidentified
ARDEVRGYLDV

SEQ ID NO: 73 - protein, 6 aa, unidentified
QGISNY

SEQ ID NO: 74 - skipped

SEQ ID NO: 75 - protein, 9 aa, unidentified
QQYSKLPYT

SEQ ID NO: 76 - protein, 5 aa, unidentified
GGGGS

SEQ ID NO: 77 - protein, 15 aa, unidentified
GGGGSGGGGSGGGGS

SEQ ID NO: 78 - protein, 20 aa, unidentified
GGGGSGGGGSGGGGSGGGGS